SEQUENCE LISTING (44 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 233 aa, Macaca fascicularis
MRKTRLWGLLWMLFVSELRATTELTEEKYEYKEGQTLEVKCDYALEKYANSRKAWQKMEGKMPKILAKTERPSENSHPVQVGRITLEDYPDHGLLQVRMTNLQVEDSGLYQCVIYQHPKESHVLFNPICLVVTKGSSGTPGSSENSTQNVYRTPSTTAKALGPRYTSPRTVTQAPPESTVVVSTPGSEINLTNVTDIIRVPVFNIVIIVAGGFLSKSLVFSVLFAVTLRSFGP

SEQ ID NO: 3 - protein, 230 aa, Mus musculus
MRKAGLWGLLCVFFVSEVKAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGSSDVFTPVIIPITRLTERPILITTKYSPSDTTTTRSLPKPTAVVSSPGLGVTIINGTDADSVSTSSVTISVICGLLSKSLVFIILFIVTKRTFG

SEQ ID NO: 4 - protein, 238 aa, Canis lupus
MRKARLWELLWLLFISELQATTEPDEIKYVLAEGGTLNMKCTTSTWKYTYSQKAWQKLMDREKPLTLIFTENVSGDTSQVQRGRYFLEDIPSEAILNVQMTNLQVEDSGLYQCVIYHPQKNPDILYPRVRLVVTKGITASDKSPTQNLAQISTHPPTTTKAQSTLLASPRTVTQLPPKSTADTSSPDFGVNITNVTNVTSYGFRFSVINIVILVLCGFLSKSLVFTVLIAVTQRSFGP

SEQ ID NO: 5 - protein, 239 aa, Felis catus
MKKAGLWGLLWMVFISELQAENESKEEKFIKEGETLNVNCPFSTEKYSYSQKAWQKLIDGRKPVTLALTTSTSGNPSQVREGRYFLEDIPSEAILHVQMTNLQVEDSGLYRCVIYYPSKEPDVLSPLFHVVVIKDPLNTTASDENHTRKLQISTFPPTTTRAQGTLVDRPRTVTKLPPKSTADLSSPGFGVNITNVTDVTSYVFRVSVTSIIIIVLCGVLSKSLVFTVLLAVTQRSFGP

SEQ ID NO: 6 - protein, 226 aa, Rattus norvegicus
MRKAGLWGLLLVFFVSEVKAAIVPEEERYDLVEGQTLTVNCPFNIMKYARSRKAWQRLSAGKEPLTLVVTERSSTTSSEVRVGKYTLKDDPTEAMLFVQMTDLQVTDSGLYRCVIYHPPNDPVLLFHPVRLVVTKGSSGVSVPDIIPTTKPTEVPVLITTKHSTPTRSLPKSTAVVSSPDPGVTINNGTDPTSVSTYNVVVPVVCGLLIKTLIFFVLFVVTKRSFG

SEQ ID NO: 7 - protein, 17 aa, synthetic construct
LQVTDSGLYRCVIYHPP

SEQ ID NO: 8 - protein, 7 aa, synthetic construct
RGFFRGG

SEQ ID NO: 9 - protein, 10 aa, synthetic construct
ARNVQHYHMK

SEQ ID NO: 10 - protein, 9 aa, synthetic construct
GLLSKSLVF

SEQ ID NO: 11 - protein, 7 aa, synthetic construct
LSKSLVF

SEQ ID NO: 12 - protein, 17 aa, synthetic construct
LQEEDAGEYGCMVDGAR

SEQ ID NO: 13 - protein, 12 aa, synthetic construct
LQEEDAGEYGCM

SEQ ID NO: 14 - protein, 117 aa, synthetic construct
EVQLVESGGVLVQPGGSLRLSCAASGITFSNSWMSWVRQAPGKGLEWVANIKQDGSEKYYVDSVKGRFTISRDNAKESLFLQMNSLRAEDTAVYYCARGGISTFDIWGQGTMVTVSS

SEQ ID NO: 15 - protein, 8 aa, synthetic construct
GITFSNSW

SEQ ID NO: 16 - protein, 8 aa, synthetic construct
IKQDGSEK

SEQ ID NO: 17 - protein, 10 aa, synthetic construct
ARGGISTFDI

SEQ ID NO: 18 - protein, 107 aa, synthetic construct
EILMTQSPATLSVSPGERATLSCRASQSVSSNLAWYQQKPGQAPRLLIYGASIRATGIPARFSGSGSRTEFTLTISSLQSEDFAVYYCQQYKNWMYTFGQGTKLEIK

SEQ ID NO: 19 - protein, 6 aa, synthetic construct
QSVSSN

SEQ ID NO: 20 - protein, 9 aa, synthetic construct
QQYKNWMYT

SEQ ID NO: 21 - protein, 25 aa, synthetic construct
EVQLVESGGVLVQPGGSLRLSCAAS

SEQ ID NO: 22 - protein, 17 aa, synthetic construct
MSWVRQAPGKGLEWVAN

SEQ ID NO: 23 - protein, 38 aa, synthetic construct
YYVDSVKGRFTISRDNAKESLFLQMNSLRAEDTAVYYC

SEQ ID NO: 24 - protein, 11 aa, synthetic construct
WGQGTMVTVSS

SEQ ID NO: 25 - protein, 26 aa, synthetic construct
EILMTQSPATLSVSPGERATLSCRAS

SEQ ID NO: 26 - protein, 17 aa, synthetic construct
LAWYQQKPGQAPRLLIY

SEQ ID NO: 27 - protein, 36 aa, synthetic construct
IRATGIPARFSGSGSRTEFTLTISSLQSEDFAVYYC

SEQ ID NO: 28 - protein, 10 aa, synthetic construct
FGQGTKLEIK

SEQ ID NO: 29 - protein, 330 aa, Homo sapiens [sequence omitted]

SEQ ID NO: 30 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 446 aa, synthetic construct
EVQLVESGGVLVQPGGSLRLSCAASGITFSNSWMSWVRQAPGKGLEWVANIKQDGSEKYYVDSVKGRFTISRDNAKESLFLQMNSLRAEDTAVYYCARGGISTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Homo sapiens [sequence omitted]

SEQ ID NO: 33 - protein, 214 aa, synthetic construct
EILMTQSPATLSVSPGERATLSCRASQSVSSNLAWYQQKPGQAPRLLIYGASIRATGIPARFSGSGSRTEFTLTISSLQSEDFAVYYCQQYKNWMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 4 aa, synthetic construct
ELLG

SEQ ID NO: 35 - protein, 17 aa, synthetic construct
NVTDIIRGGGGSGLNDI

SEQ ID NO: 36 - protein, 8 aa, synthetic construct
DVKCDYTL

SEQ ID NO: 37 - protein, 186 aa, Homo sapiens
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFN

SEQ ID NO: 38 - DNA, 351 bp, synthetic construct
  source 1..351: mol_type = other DNA
gaagtgcagctggtggagtctgggggggtgctcgtccaacccggcggcagcctccgactgagctgcgctgcctccggaataactttctccaactcctggatgagctgggttcgccaagcacccggcaagggcctggaatgggtggccaatattaaacaggatggctctgaaaagtattatgtggactctgtaaaaggaaggttcacaatttcacgagacaatgccaaggaatccctgttcctgcaaatgaactccttgcgggcagaggatactgctgtttattactgtgctaggggaggaatcagcacattcgatatctgggggcaaggcaccatggtcactgtgtcatct

SEQ ID NO: 39 - DNA, 321 bp, synthetic construct
  source 1..321: mol_type = other DNA
gagatccttatgacccaatcaccagccaccctgagtgtctcccccggagaacgcgcaacactgtcctgtcgggccagccagtccgtatcctctaatttggcttggtatcagcaaaagccaggacaggccccacgacttctgatctatggcgcatctataagggcaacaggaatacccgctagattttctgggtcagggagcagaaccgagttcaccctgaccatttccagcctccagtccgaggactttgctgtctactactgtcaacaatataagaactggatgtatacatttggccaaggcaccaagctggagattaaa

SEQ ID NO: 40 - protein, 156 aa, Homo sapiens [sequence omitted]

SEQ ID NO: 41 - protein, 200 aa, synthetic construct
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRGGGGSGLNDIFEAQKIEAHE

SEQ ID NO: 42 - DNA, 1344 bp, synthetic construct
  source 1..1344: mol_type = other DNA
gaagtgcagctggtggagtctgggggggtgctcgtccaacccggcggcagcctccgactgagctgcgctgcctccggaataactttctccaactcctggatgagctgggttcgccaagcacccggcaagggcctggaatgggtggccaatattaaacaggatggctctgaaaagtattatgtggactctgtaaaaggaaggttcacaatttcacgagacaatgccaaggaatccctgttcctgcaaatgaactccttgcgggcagaggatactgctgtttattactgtgctaggggaggaatcagcacattcgatatctgggggcaaggcaccatggtcactgtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctcccgtggctgggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaataatag

SEQ ID NO: 43 - DNA, 648 bp, synthetic construct
  source 1..648: mol_type = other DNA
gagatccttatgacccaatcaccagccaccctgagtgtctcccccggagaacgcgcaacactgtcctgtcgggccagccagtccgtatcctctaatttggcttggtatcagcaaaagccaggacaggccccacgacttctgatctatggcgcatctataagggcaacaggaatacccgctagattttctgggtcagggagcagaaccgagttcaccctgaccatttccagcctccagtccgaggactttgctgtctactactgtcaacaatataagaactggatgtatacatttggccaaggcaccaagctggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatag

SEQ ID NO: 44 - protein, 238 aa, Sus scrofa
MRSARLGRLLWMLFITEIQAATELPEEKYILAEGETLNVNCPVTVGVYSNSRKAWQKLNRNGKFQTLAITERVSGQVSKVQVGKIFLTDEPSEGMLHVQMTNVQAEDSGLYRCVIYQPPKDPIILFYPVRLVVTNYSSGTPASAETPTQSCSPTTTLPPTTTTNRHRPRPRTVRTVTQFLTDFTTSLSSPGLKVTLTNVTDITRDTEISLILPAVCGLLSKSLVFIVLFVVTRMSFTP

(19) United States
(12) Patent Application Publication
(10) Pub. No.: US 2026/0242465 A1
(43) Pub. Date: Aug. 20, 2026

(54) ANTI-TREM1 ANTIBODY AGENTS, COMPOSITIONS, AND USES THEREOF

(71) Applicant: Celsius Therapeutics, Inc., Cambridge, MA (US)

(72) Inventors: Marwa Bsat, West Roxbury, MA (US); Juan Lopez Fiallo, Boston, MA (US); Siyang Guo, Wakefield, MA (US); Gerard Stefan Honig, Kingston, NY (US); Kristen Johnson, Cambridge, MA (US); Ping Li, Wellesley, MA (US); Lawrence L'Italien, Boston, MA (US); Jeanne Sue Magram, Croton On Hudson, NY (US); Nicolas Stransky, Cambridg, MA (US); Mukund Madhav Varma, Medford, MA (US); Stephanie Wiltzius, Winchester, MA (US); Sarah Zuerndorfer, Milton, MA (US)

(21) Appl. No.: 18/993,126

(22) PCT Filed: Apr. 21, 2024
(86) PCT No.: PCT/US2024/025612

§ 371 Date: Jan. 10, 2025

Related U.S. Application Data
(60) Provisional application No. 63/461,130, filed on Apr. 21, 2023.

(51) Int. Cl. C07K 16/28; A61P 37/06

(52) U.S. Cl. CPC C07K 16/2803; A61P 37/06

(57) ABSTRACT

Provided herein are antibody agents that bind specifically to TREM1, nucleic acids encoding the same, compositions comprising the same, and methods of making and using the same.

### CROSS-REFERENCE TO RELATED APPLICATION

The present application claims priority to U.S. Provisional Application No. 63/461,130, filed Apr. 21, 2023, the entirety of which is incorporated herein by reference.

### BACKGROUND

The incidence of immune-mediated inflammatory disease (IMID) in western civilizations has been reported to be in the range of 5-7%. A variety of therapeutic strategies have been pursued, but failure rates remain high.

### SUMMARY

The present disclosure provides, among other things, particularly useful antibody agents (e.g., antibodies, antigen-binding portions thereof, nucleic acids encoding the same, and entities that include them) that bind to Triggering Receptor Expressed on Myeloid Cells 1 (TREM1).

The present disclosure provides an insight that levels of particular cell types and/or of product(s) (e.g., proteins, etc.) produced by particular cell types in certain subjects (e.g., in sample(s) from such subjects) can be indicative of likely responsiveness to anti-inflammatory therapies (e.g., to an anti-TREM1 antibody agent or a nucleic acid encoding the same).

We note that certain therapeutic strategies for addressing inflammation focus on specific targets other than TREM1 (e.g., TNFα) (see, for example, Sakemi et al., Medicine 99:e23344, 2020). Certain patients may be less responsive or non-responsive to such alternative therapies. For example, response rates for certain TNFα therapies are often reported as less than 50%. Without wishing to be bound by any particular theory, the present disclosure provides anti-TREM1 antibody agents and nucleic acids encoding the same described herein for use in the treatment of subjects having an inflammatory disease, disorder or condition who are relatively less likely to respond to alternative therapies (e.g., therapies targeting TNFα) or in whom continued such therapy may not be effective, or may decrease in effectiveness, and/or who may be relatively more likely to respond to different therapies (e.g., to therapy targeting TREM1).

In at least one aspect, the present disclosure provides an antibody agent that binds to human triggering receptor expression on myeloid cells 1 (TREM1), wherein the antibody comprises: (a) a light chain having CDR1, CDR2, and CDR3 sequences that, collectively, have at least 85%, 90%, 95%, 96%, 97%, 98%, or 99% identity to those found in SEQ ID NO: 18, and/or include not more than 1, 2, 3, 4, or 5 amino acid sequence differences relative to those found in SEQ ID NO: 18; and/or (b) a heavy chain having CDR1, CDR2, and CDR3 sequences that, collectively, have at least 85%, 90%, 95%, 96%, 97%, 98%, or 99% identity to those found in SEQ ID NO: 14, and/or include not more than 1, 2, 3, 4, or 5 amino acid sequence differences relative to those found in SEQ ID NO: 14.

In some embodiments, an antibody agent of the present disclosure comprises a light chain (LC) comprising: (i) an LC CDR1 having an amino acid sequence that is or comprises: the amino acid sequence of SEQ ID NO: 19, an amino acid sequence with at least 85%, 90%, 95%, 96%, 97%, 98%, or 99% identity to SEQ ID NO: 19, or an amino acid sequence having not more than 1, 2, 3, 4, or 5 amino acid sequence differences relative to SEQ ID NO: 19; (ii) an LC CDR2 having an amino acid sequence that is or comprises: the amino acid sequence of GAS, or an amino acid sequence having not more than 1 or 2 amino acid sequence differences relative to the amino acid sequence of GAS; and (iii) an LC CDR3 having an amino acid sequence that is or comprises: the amino acid sequence of SEQ ID NO: 20, an amino acid sequence with at least 85%, 90%, 95%, 96%, 97%, 98%, or 99% identity to SEQ ID NO: 20, or an amino acid sequence having not more than 1, 2, 3, 4, or 5 amino acid sequence differences relative to SEQ ID NO: 20.

In some embodiments, an antibody agent of the present disclosure comprises a heavy chain (HC) comprising: (i) an HC CDR1 having an amino acid sequence that is or comprises: the amino acid sequence of SEQ ID NO: 15, an amino acid sequence with at least 85%, 90%, 95%, 96%, 97%, 98%, or 99% identity to SEQ ID NO: 15, or an amino acid sequence having not more than 1, 2, 3, 4, or 5 amino acid sequence differences relative to SEQ ID NO: 15; (ii) an HC CDR2 having an amino acid sequence that is or comprises: the amino acid sequence of SEQ ID NO: 16, an amino acid sequence with at least 85%, 90%, 95%, 96%, 97%, 98%, or 99% identity to SEQ ID NO: 16, or an amino acid sequence having not more than 1, 2, 3, 4, or 5 amino acid sequence differences relative to SEQ ID NO: 16; and (iii) an HC CDR3 having an amino acid sequence that is or comprises: the amino acid sequence of SEQ ID NO: 17, an amino acid sequence with at least 85%, 90%, 95%, 96%, 97%, 98%, or 99% identity to SEQ ID NO: 17, or an amino acid sequence having not more than 1, 2, 3, 4, or 5 amino acid sequence differences relative to SEQ ID NO: 17.

In some embodiments, an antibody agent of the present disclosure comprises a light chain comprising a variable region (VL) comprising one, two, three, or four FR regions that, collectively, have at least 85%, 90%, 95%, 96%, 97%, 98%, or 99% identity to those found in SEQ ID NO: 18; and/or include not more than 1, 2, 3, 4, or 5 amino acid sequence differences relative to those found in SEQ ID NO: 18. In some embodiments, the light chain comprises a variable region (VL) comprising one, two, three, or four FR regions that each independently has an amino acid sequence that is or comprises the amino acid sequence of one of SEQ ID NOs: 25, 26, 27, or 28, an amino acid sequence with at least 85%, 90%, 95%, 96%, 97%, 98%, or 99% identity to one of SEQ ID NOs: 25, 26, 27, or 28, or an amino acid sequence having not more than 1, 2, 3, 4, or 5 amino acid sequence differences relative to one of SEQ ID NOs: 25, 26, 27, or 28.

In some embodiments, an antibody agent of the present disclosure comprises: (i) an amino acid sequence that is or comprises: the amino acid sequence of SEQ ID NO: 25, an amino acid sequence with at least 85%, 90%, 95%, 96%, 97%, 98%, or 99% identity to SEQ ID NO: 25, or an amino acid sequence having not more than 1, 2, 3, 4, or 5 amino acid sequence differences relative to SEQ ID NO: 25; and/or (ii) an amino acid sequence that is or comprises: the amino acid sequence of SEQ ID NO: 26, an amino acid sequence with at least 85%, 90%, 95%, 96%, 97%, 98%, or 99% identity to SEQ ID NO: 26, or an amino acid sequence having not more than 1, 2, 3, 4, or 5 amino acid sequence differences relative to SEQ ID NO: 26; and/or (iii) an amino acid sequence that is or comprises: the amino acid sequence of SEQ ID NO: 27, an amino acid sequence with at least 85%, 90%, 95%, 96%, 97%, 98%, or 99% identity to SEQ ID NO: 27, or an amino acid sequence having not more than 1, 2, 3, 4, or 5 amino acid sequence differences relative to SEQ ID NO: 27; and/or (iv) an amino acid sequence that is or comprises: the amino acid sequence of SEQ ID NO: 28, an amino acid sequence with at least 85%, 90%, 95%, 96%, 97%, 98%, or 99% identity to SEQ ID NO: 28, or an amino acid sequence having not more than 1, 2, 3, 4, or 5 amino acid sequence differences relative to SEQ ID NO: 28.

In some embodiments, an antibody agent of the present disclosure comprises a light chain that comprises a sequence for a constant region (CL). In some embodiments, the light chain comprises a kappa CL or a lambda CL. In some embodiments, the light chain comprises a kappa CL. In some embodiments, the light chain comprises an amino acid sequence that is or comprises the amino acid sequence of SEQ ID NO: 32, an amino acid sequence with at least 85%, 90%, 95%, 96%, 97%, 98%, or 99% identity to SEQ ID NO: 32, or an amino acid sequence having not more than 5, 10, or 20 amino acid sequence differences relative to SEQ ID NO: 32.

In some embodiments, an antibody agent of the present disclosure comprises a heavy chain comprising a variable region (VH) comprising one, two, three, or four FR regions that, collectively, have at least 85%, 90%, 95%, 96%, 97%, 98%, or 99% identity to those found in SEQ ID NO: 14, and/or include not more than 1, 2, 3, 4, or 5 amino acid sequence differences relative to those found in SEQ ID NO: 14. In some embodiments, the heavy chain comprises a variable region (VH) comprising one, two, three, or four FR regions that each independently has an amino acid sequence that is or comprises the amino acid sequence of one of SEQ ID NOs: 21, 22, 23, or 24, an amino acid sequence with at least 85%, 90%, 95%, 96%, 97%, 98%, or 99% identity to one of SEQ ID NOs: 21, 22, 23, or 24, or an amino acid sequence having not more than 1, 2, 3, 4, or 5 amino acid sequence differences relative to one of SEQ ID NOs: 21, 22, 23, or 24.

In some embodiments, an antibody agent of the present disclosure comprises: (i) an amino acid sequence that is or comprises: the amino acid sequence of SEQ ID NO: 21, an amino acid sequence with at least 85%, 90%, 95%, 96%, 97%, 98%, or 99% identity to SEQ ID NO: 21, or an amino acid sequence having not more than 1, 2, 3, 4, or 5 amino acid sequence differences relative to SEQ ID NO: 21; and/or (ii) an amino acid sequence that is or comprises: the amino acid sequence of SEQ ID NO: 22, an amino acid sequence with at least 85%, 90%, 95%, 96%, 97%, 98%, or 99% identity to SEQ ID NO: 22, or an amino acid sequence having not more than 1, 2, 3, 4, or 5 amino acid sequence differences relative to SEQ ID NO: 22; and/or (iii) an amino acid sequence that is or comprises: the amino acid sequence of SEQ ID NO: 23, an amino acid sequence with at least 85%, 90%, 95%, 96%, 97%, 98%, or 99% identity to SEQ ID NO: 23, or an amino acid sequence having not more than 1, 2, 3, 4, or 5 amino acid sequence differences relative to SEQ ID NO: 23; and/or (iv) an amino acid sequence that is or comprises: the amino acid sequence of SEQ ID NO: 24, an amino acid sequence with at least 85%, 90%, 95%, 96%, 97%, 98%, or 99% identity to SEQ ID NO: 24, or an amino acid sequence having not more than 1, 2, 3, 4, or 5 amino acid sequence differences relative to SEQ ID NO: 24.

In some embodiments, an antibody agent of the present disclosure comprises a heavy chain region that comprises a sequence for at least one constant region (CH). In some embodiments, the at least one constant region comprises an Fc domain. In some embodiments, the Fc domain comprises a mouse, a rat, a rabbit, a primate, a human, a dog, a pig, or a cat Fc domain. In some embodiments, the Fc domain is selected from an Fc domain of an immunoglobulin isotype. In some embodiments, the immunoglobulin isotype comprises IgA, IgG, IgM, or IgE. In some embodiments, the Fc domain comprises an Fc domain of an IgG. In some embodiments, the Fc domain comprises an Fc domain of a human IgG. In some embodiments, the IgG is or comprises IgG1, IgG2, IgG3, or IgG4. In some embodiments, the IgG constant region comprises one or more modifications. In some embodiments, the one or more modifications modulates one or more properties of the antibody agent. In some embodiments, the one or more modifications comprises a PVAdelG mutation. In some embodiments, the heavy chain comprises an amino acid sequence that is or comprises the amino acid sequence of SEQ ID NO: 30, an amino acid sequence with at least 85%, 90%, 95%, 96%, 97%, 98%, or 99% identity to SEQ ID NO: 30, or an amino acid sequence having not more than 5, 10, or 20 amino acid sequence differences relative to SEQ ID NO: 30.

In some embodiments, an antibody agent of the present disclosure comprises a VL having an amino acid sequence that is or comprises: the amino acid sequence of SEQ ID NO: 18, an amino acid sequence with at least 85%, 90%, 95%, 96%, 97%, 98%, or 99% identity to SEQ ID NO: 18, or an amino acid sequence having not more than 5, 10, or 20 amino acid sequence differences relative to SEQ ID NO: 18. In some embodiments, an antibody agent of the present disclosure comprises a VH having an amino acid sequence that is or comprises: the amino acid sequence of SEQ ID NO: 14, an amino acid sequence with at least 85%, 90%, 95%, 96%, 97%, 98%, or 99% identity to SEQ ID NO: 14, or an amino acid sequence having not more than 5, 10, or 20 amino acid sequence differences relative to SEQ ID NO: 14.

In some embodiments, an antibody agent of the present disclosure comprises a light chain (LC) having an amino acid sequence that is or comprises the amino acid sequence of SEQ ID NO: 33, an amino acid sequence with at least 85%, 90%, 95%, 96%, 97%, 98%, or 99% identity to SEQ ID NO: 33, or an amino acid sequence having not more than 5, 10, or 20 amino acid sequence differences relative to SEQ ID NO: 33. In some embodiments, an antibody agent of the present disclosure comprises a heavy chain (HC) having an amino acid sequence that is or comprises the amino acid sequence of SEQ ID NO: 31, an amino acid sequence with at least 85%, 90%, 95%, 96%, 97%, 98%, or 99% identity to SEQ ID NO: 31, or an amino acid sequence having not more than 5, 10, or 20 amino acid sequence differences relative to SEQ ID NO: 31.

In at least one aspect, the present disclosure provides an antibody agent that is characterized by one or more of: (i) the antibody agent does not bind to or has minimal binding affinity for TREM2; (ii) the antibody agent competes for binding with a reference antibody agent that binds to TREM1; (iii) the antibody agent binds to monomeric human TREM1-ECD protein with a binding affinity (K_{D}) of about 0.1 nM to about 0.3 nM; (iv) the antibody agent binds to monomeric cyno TREM1-ECD protein with a binding affinity (K_{D}) of about 1.4 nM to about 2.4 nM; (v) the antibody agent binds to human TREM1 in an enriched human monocyte cell binding assay with a binding affinity (K_{D}) of about 80 pM to about 95 pM; (vi) the antibody agent binds to human TREM1 in an enriched human neutrophil cell binding assay with a binding affinity (KD) of about 185 pM to about 245 pM; (vii) the antibody agent binds to human TREM1 in a whole blood human monocyte cell binding assay with a binding affinity (K_{D}) of about 90 pM to about 115 pM; (viii) the antibody agent binds to human TREM1 in a whole blood human neutrophil cell binding assay with a binding affinity (K_{D}) of about 110 pM to about 150 pM; (ix) the antibody agent binds to cyno TREM1 in a whole blood cyno neutrophil cell binding assay with a binding affinity (K_{D}) of about 235 pM to about 370 pM; (x) the antibody agent inhibits TREM1 activity in a human whole blood enriched neutrophil or monocyte primary cell function assay with an IC50 of about 20 pM to about 40 pM; (xi) the antibody agent inhibits TREM1 activity in a cyno whole blood primary cell function assay with an IC50 of about 4 pM to about 13 pM; (xii) the antibody agent can be produced at a concentration of about 4 g/L to about 7 g/L; (xiii) the antibody agent has a melting temperature of about 70° C. to about 80° C.; (xiv) the antibody agent can bind to non-activated TREM1 and activated TREM1; (xv) the antibody agent does not bind to the surface of cells that do not express TREM1; or (xvi) the antibody agent antagonizes TREM1-mediated inhibition of maturation of monocytes to macrophages.

In some embodiments, an antibody agent of the present disclosure is characterized in that it reduces TREM1 activity and/or cell surface level relative to a comparator. In some embodiments, the comparator is or comprises: (i) a sample that is not contacted with a TREM1 antibody agent disclosed herein; or (ii) a sample that is contacted with a reference TREM1 antibody agent; or (iii) a sample that is contacted with an isotype control antibody agent. In some embodiments, the antibody agent reduces the level of TREM1 present on the surface of a cell. In some embodiments, the cell is a monocyte or a neutrophil. In some embodiments, the antibody agent reduces the level of TREM1 present on the surface of a cell by internalization and/or clipping of TREM1. In some embodiments, the antibody agent reduces (e.g., inhibits) TREM1 activity. In some embodiments, inhibition of TREM1 activity comprises inhibiting binding of TREM1 to a TREM1 ligand. In some embodiments, the TREM1 ligand is PGLYRP1. In some embodiments, the antibody agent reduces the activity and/or level of TREM1 by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, the antibody agent is characterized in that it reduces (e.g., inhibits) a TREM1 mediated immune response. In some embodiments, the TREM1 mediated immune response is an amplified immune response and/or is not an innate immune response (e.g., a TLR mediated immune response). In some embodiments, the TREM1 mediated immune response is mediated by monocytes (e.g., inflammatory monocytes) and/or neutrophils (e.g., activated neutrophils). In some embodiments, the TREM1 mediated immune response is or comprises one or more of: (i) secretion of one or more cytokines; (ii) secretion of one or more chemokines; (iii) modulation of T cells, B cells, or other cells; (iv) secretion of one or more factors that reduce or impair epithelial barrier integrity; and (v) secretion of one or more proteolytic enzymes. In some embodiments, the one or more cytokines are proinflammatory cytokines. In some embodiments, the one or more cytokines are selected from the group consisting of: IL-1α, IL-1β, IL-6, IL-8, IL-10, IL-23, GM-CSF, TNF-RII, and TNFα. In some embodiments, the one or more chemokines are selected from the group consisting of: CCL2, CCL3, CCL4, CCL8, CCL20, CCL22, CCL24, CXCL1, CXCL5, CXCL9, CXCL10, and CXCL13. In some embodiments, the modulation of T cells, B cells, or other cells is by one or more of IL-1α, IL-1β, IL-6, IL-10, IL-23, APRIL, BAFF, CD30, M-CSF, TNF-RII, and TNFα. In some embodiments, the one or more factors that reduce or impair epithelial barrier integrity are selected from the group consisting of: IL-1α, IL-1β, IL-6, IL-8, IL-10, IL-23, GM-CSF, TRAIL, TWEAK, MMP-1, IL-20, TNFR-II, and TNFα. In some embodiments, the one or more proteolytic enzymes is or comprises a matrix metalloproteinase (MMP). In some embodiments, the MMP is MMP1 and/or MMP9.

In some embodiments, an antibody agent of the present disclosure is characterized in that it prevents or reduces (e.g., inhibits) epithelial barrier damage. In some embodiments, the epithelial barrier damage is mediated by one or more cytokines. In some embodiments, the one or more cytokines are proinflammatory cytokines. In some embodiments, the one or more cytokines are selected from the group consisting of: IL-1β, IL-6, IL-8, IL-23, and TNFα.

In some embodiments, an antibody agent of the present disclosure is or comprises: (i) an intact IgA, IgG, IgD, IgE, or IgM antibody; (ii) an antibody fragment; (iii) a single domain antibody; (iv) a single chain Fv; or (v) a polypeptide comprising antigen binding specificity fused to a Fc domain. In some embodiments, the antibody agent is an antibody-drug conjugate (ADC). In some embodiments, the antibody agent further comprises a second binding specificity. In some embodiments, the second binding specificity confers binding to an antigen other than human TREM1. In some embodiments, the antibody agent is selected from: a heterodimer, a Crossmab, a DVD-Ig, a 2-in-1 IgG, and IgG-sc-Fv, an scFv-scFv, a BiTE, a DART, a diabody, a Fab-scFv fusion, a Fab-Fab fusion, or a tandem antibody.

In some embodiments, an antibody agent of the present disclosure is produced in a bacterial cell, a yeast cell, an insect cell, or a mammalian cell. In some embodiments, the antibody agent is produced in a mammalian cell. In some embodiments, the mammalian cell line is or comprises CHO cells. In some embodiments, the antibody agent is produced in a cell-free system.

In at least one aspect, the present disclosure provides a polypeptide having an amino acid sequence that is or comprises: (i) LC CDR1, LC CDR2, and LC CDR3 sequences that, collectively, have at least 85%, 90%, 95%, 96%, 97%, 98%, or 99% identity to those found in SEQ ID NO: 18, and/or include not more than 1, 2, 3, 4, or 5 amino acid sequence differences relative to those found in SEQ ID NO: 18; (ii) the amino acid sequence of SEQ ID NO: 18, an amino acid sequence with at least 85%, 90%, 95%, 96%, 97%, 98%, or 99% identity to SEQ ID NO: 18, or an amino acid sequence having not more than 5, 10, or 20 amino acid sequence differences relative to SEQ ID NO: 18; and/or (iii) the amino acid sequence of SEQ ID NO: 33, an amino acid sequence with at least 85%, 90%, 95%, 96%, 97%, 98%, or 99% identity to SEQ ID NO: 33, or an amino acid sequence having not more than 5, 10, or 20 amino acid sequence differences relative to SEQ ID NO: 33.

In at least one aspect, the present disclosure provides a polypeptide having an amino acid sequence that is or comprises: (i) HC CDR1, HC CDR2, and HC CDR3 sequences that, collectively, have at least 85%, 90%, 95%, 96%, 97%, 98%, or 99% identity to those found in SEQ ID NO: 14, and/or include not more than 1, 2, 3, 4, or 5 amino acid sequence differences relative to those found in SEQ ID NO: 14; (ii) the amino acid sequence of SEQ ID NO: 14, an amino acid sequence with at least 85%, 90%, 95%, 96%, 97%, 98%, or 99% identity to SEQ ID NO: 14, or an amino acid sequence having not more than 5, 10, or 20 amino acid sequence differences relative to SEQ ID NO: 14; and/or (iii) the amino acid sequence of SEQ ID NO: 31, an amino acid sequence with at least 85%, 90%, 95%, 96%, 97%, 98%, or 99% identity to SEQ ID NO: 31, or an amino acid sequence having not more than 5, 10, or 20 amino acid sequence differences relative to SEQ ID NO: 31.

In at least one aspect, the present disclosure provides a polypeptide having an amino acid sequence that is or comprises: (i) LC CDR1, LC CDR2, and LC CDR3 sequences that, collectively, have at least 85%, 90%, 95%, 96%, 97%, 98%, or 99% identity to those found in SEQ ID NO: 18, and/or include not more than 1, 2, 3, 4, or 5 amino acid sequence differences relative to those found in SEQ ID NO: 18; and (ii) HC CDR1, HC CDR2, and HC CDR3 sequences that, collectively, have at least 85%, 90%, 95%, 96%, 97%, 98%, or 99% identity to those found in SEQ ID NO: 14, and/or include not more than 1, 2, 3, 4, or 5 amino acid sequence differences relative to those found in SEQ ID NO: 14.

In at least one aspect, the present disclosure provides a nucleic acid having a nucleotide sequence that encodes an antibody agent described herein. In some embodiments, the nucleic acid has a nucleotide sequence that encodes a polypeptide described herein. In some embodiments, the nucleic acid has a nucleotide sequence that encodes a variable light chain (VL), wherein the nucleotide sequence is or comprises: the nucleotide sequence of SEQ ID NO: 39, or a nucleotide sequence with at least 85%, 90%, 95%, 96%, 97%, 98%, or 99% identity to SEQ ID NO: 39. In some embodiments, the nucleic acid has a nucleotide sequence that encodes a variable heavy chain (VH), wherein the nucleotide sequence is or comprises: the nucleotide sequence of SEQ ID NO: 38, or a nucleotide sequence with at least 85%, 90%, 95%, 96%, 97%, 98%, or 99% identity to SEQ ID NO: 38. In some embodiments, the nucleic acid has a nucleotide sequence that encodes an antibody agent that binds to TREM1, wherein: (i) the nucleotide sequence comprises a first portion encoding a variable light chain (VL), wherein the first portion is or comprises: the nucleotide sequence of SEQ ID NO: 39, or a nucleotide sequence with at least 85%, 90%, 95%, 96%, 97%, 98%, or 99% identity to SEQ ID NO: 39; and (ii) a nucleotide sequence comprises a second portion encoding a variable heavy chain (VH), wherein the second portion is or comprises: the nucleotide sequence of SEQ ID NO: 38, or a nucleotide sequence with at least 85%, 90%, 95%, 96%, 97%, 98%, or 99% identity to SEQ ID NO: 38. In some embodiments, the antibody agent encoded by the nucleotide sequence further comprises a light chain constant region (CL) and/or at least one heavy chain constant region.

In at least one aspect, the present disclosure provides a vector comprising a nucleic acid described herein.

In at least one aspect, the present disclosure provides a host cell comprising a vector described herein. In some embodiments, the host cell is a yeast cell, a bacterial cell, a mammalian cell, or an insect cell.

In at least one aspect, the present disclosure provides a method of making an antibody agent that binds to TREM1, the method comprising: culturing a host cell described herein under a condition wherein an antibody agent described herein is expressed by said host cell.

In at least one aspect, the present disclosure provides a composition comprising an antibody agent described herein. In some embodiments, the composition comprises a polypeptide described herein.

In at least one aspect, the present disclosure provides a pharmaceutical composition that comprises or delivers an antibody agent described herein. In some embodiments, the pharmaceutical composition comprises or delivers a polypeptide described herein. In some embodiments, the pharmaceutical composition further comprises an excipient and/or a pharmaceutically acceptable carrier. In some embodiments, the pharmaceutical composition is formulated in one or more unit dosage forms.

In at least one aspect, the present disclosure provides a method of making a pharmaceutical composition, the method comprising a step of: combining an antibody agent described herein, a polypeptide described herein, or a nucleic acid that encodes part or all thereof, with one or more pharmaceutically acceptable carriers.

In at least one aspect, the present disclosure provides a method of treating a disease, disorder, or condition associated with a TREM1 mediated immune response in a subject, the method comprising a step of: administering to the subject a pharmaceutical composition described herein. In some embodiments, the pharmaceutical composition is characterized in that when administered to the subject it reduces the level and/or activity of TREM1 relative to a comparator. In some embodiments, the comparator comprises an otherwise similar subject not administered the pharmaceutical composition, or administered a reference TREM1 inhibitor.

In some embodiments, a disease, disorder, or condition is an inflammatory disease, disorder, or condition. In some embodiments, the inflammatory disease, disorder, or condition is selected from: an inflammatory bowel disease (IBD), sepsis, a fibrotic disease, rheumatoid arthritis, chronic obstructive pulmonary disease (COPD), acute respiratory distress syndrome (ARDS), COVID-19, Post-MI (ischemic reperfusion), atherosclerosis, acute stroke (ischemic reperfusion), stroke (hemorrhagic), renal ischemia reperfusion injury, pancreatitis, renal fibrosis, liver fibrosis, NASH, Sickle cell vaso-occlusive crisis, Marfan syndrome, HIV infection, motor neurogenerative disorders, periodontal/gingivitis, cancers, diabetic foot ulcers, gout, lupus, psoriasis, an arthropathy (e.g., arthritis or synovitis), and Behcet's disease. In some embodiments, the disease, disorder, or condition is an inflammatory bowel disease (IBD) (e.g., intestinal IBD or an extra-intestinal manifestation (EIM) of IBD). In some embodiments, the IBD is Crohn's disease (CD) or ulcerative colitis (UC). In some embodiments, the disease, disorder, or condition is non-responsive to an alternative therapy. In some embodiments, the alternative therapy is or comprises anti-TNFα therapy.

In some embodiments, a subject has received, or is receiving, an alternative therapy. In some embodiments, the alternative therapy is an alternative IBD therapy. In some embodiments, alternative therapy is or comprises an anti-TNFα therapy (e.g., infliximab, adalimumab, golimumab, and certolizumab pegol); a JAK inhibitor therapy (including selective and non-selective inhibitors) (e.g., upadacitinib, tofacitinib and filgotinib); an anti-integrin therapy (e.g., vedolizumab and natalizumab); an anti-IL-23 therapy (e.g., guselkumab, mirikizumab, and brazikumab); an anti-IL-12/23 therapy (e.g., ustekinumab); an anti-IL-23A therapy (e.g., risankizumab); an S1PR agonist or modulator therapy (e.g., ozanimod and etrasimod); a 5-aminosalicylate therapy (e.g., mesalamine, olsalazine, balsalazide, and sulfasalazine); an immunomodulator therapy (e.g., azathioprine, 6-mercaptopurine, and Methotrexate); a corticosteroid therapy (e.g., prednisone, methylprednisolone, hydrocortisone, and budesonide); an anti-TL1A therapy (e.g., PRA023 and RVT-3101); a kinase inhibitor therapy (e.g., ritlecitinib), a TYK2 inhibitor therapy (e.g., deucravacitinib), an anti-IL-36 therapy (e.g., spesolimab); an anti-IL-13 therapy (e.g., dupilumab), a miR-124 upregulator therapy (e.g., obefazimod), a TLR9 agonist therapy (e.g., cobitolimod); or a combination thereof.

In some embodiments, a subject has been determined to express a biomarker that: (i) is or is a proxy for an elevated level of neutrophils and/or monocytes (e.g., inflammatory monocytes); (ii) is or comprises one or more product(s) of neutrophils and/or monocytes (e.g., inflammatory monocytes), wherein the biomarker has been correlated with responsiveness to an antibody agent described herein; and/or (iii) is indicative of TREM1 level and/or activity. In some embodiments, the biomarker is or is a proxy for an elevated level of activated neutrophils. In some embodiments, the biomarker is or comprises one or more of: (i) a cell-based biomarker; (ii) a tissue biomarker; (iii) a product produced by activated neutrophils; (iv) a product produced by inflammatory monocytes; (v) a TREM1 ligand; and (vi) a TREM1 gene activation signature. In some embodiments, the cell-based biomarker is monocyte and/or neutrophil count, TREM1 RNA present in monocytes and/or neutrophils, TREM1 present in monocytes and/or neutrophils, or TREM1 present on the cell surface of monocytes and/or neutrophils. In some embodiments, the tissue biomarker is mucosal ulceration, neutrophils in lamina propria and/or epithelial cells, or lymphocytes, plasmocytes, and/or eosinophils in lamina propria. In some embodiments, the product produced by activated neutrophils is calprotectin, PGLYRP1, or soluble TREM1. In some embodiments, the product produced by inflammatory monocytes is soluble TREM1. In some embodiments, the TREM1 ligand is bacterial peptidoglycan (PGN) and/or PGLYRP1. In some embodiments, the biomarker is detected in a sample selected from the group consisting of: blood, disease tissue, fecal matter, components or fractions thereof, and combinations thereof. In some embodiments, the biomarker is monocyte and/or neutrophil count and the biomarker is detected in disease tissue. In some embodiments, the biomarker is TREM1 RNA present in monocytes and/or neutrophils and the biomarker is detected in disease tissue. In some embodiments, the biomarker is TREM1 present in monocytes and/or neutrophils and the biomarker is detected in disease tissue. In some embodiments, the biomarker is TREM1 present on the cell surface of monocytes and/or neutrophils and the biomarker is detected in disease tissue. In some embodiments, the biomarker is a tissue biomarker and the biomarker is detected in disease tissue. In some embodiments, the biomarker is soluble TREM1 and the biomarker is detected in blood and/or fecal matter. In some embodiments, the biomarker is calprotectin and the biomarker is detected in blood and/or fecal matter. In some embodiments, the biomarker is bacterial PGN and the biomarker is detected in fecal matter. In some embodiments, the biomarker is PGLYRP1 and the biomarker is detected in blood and/or fecal matter. In some embodiments, the biomarker is a TREM1 gene activation signature and the biomarker is detected in disease tissue and/or blood.

In at least one aspect, the present disclosure provides a method comprising steps of: determining whether a subject expresses a biomarker that: (i) is or is a proxy for an elevated level of neutrophils and/or monocytes (e.g., inflammatory monocytes); (ii) is or comprises one or more product(s) of neutrophils and/or monocytes (e.g., inflammatory monocytes), wherein the biomarker has been correlated with responsiveness to an antibody agent disclosed herein; and/or (iii) is indicative of TREM1 level and/or activity; and administering an antibody agent disclosed herein to the subject, if the subject is determined to express the biomarker. In some embodiments, the biomarker is or is a proxy for an elevated level of activated neutrophils. In some embodiments, the biomarker is or comprises one or more of: (i) a cell-based biomarker; (ii) a tissue biomarker; (iii) a product produced by activated neutrophils; (iv) a product produced by inflammatory monocytes; (v) a TREM1 ligand; and (vi) a TREM1 gene activation signature. In some embodiments, the cell-based biomarker is monocyte and/or neutrophil count, TREM1 RNA present in monocytes and/or neutrophils, TREM1 present in monocytes and/or neutrophils, or TREM1 present on the cell surface of monocytes and/or neutrophils. In some embodiments, the tissue biomarker is mucosal ulceration, neutrophils in lamina propria and/or epithelial cells, or lymphocytes, plasmocytes, and/or eosinophils in lamina propria. In some embodiments, the product produced by activated neutrophils is calprotectin, PGLYRP1, or soluble TREM1. In some embodiments, the product produced by inflammatory monocytes is soluble TREM1. In some embodiments, the TREM1 ligand is bacterial peptidoglycan (PGN) and/or PGLYRP1. In some embodiments, the biomarker is detected in a sample selected from the group consisting of: blood, disease tissue, fecal matter, components or fractions thereof, and combinations thereof. In some embodiments, the biomarker is monocyte and/or neutrophil count and the biomarker is detected in disease tissue. In some embodiments, the biomarker is TREM1 RNA present in monocytes and/or neutrophils and the biomarker is detected in disease tissue. In some embodiments, the biomarker is TREM1 present in monocytes and/or neutrophils and the biomarker is detected in disease tissue. In some embodiments, the biomarker is TREM1 present on the cell surface of monocytes and/or neutrophils and the biomarker is detected in disease tissue. In some embodiments, the biomarker is a tissue biomarker and the biomarker is detected in disease tissue. In some embodiments, the biomarker is soluble TREM1 and the biomarker is detected in blood and/or fecal matter. In some embodiments, the biomarker is calprotectin and the biomarker is detected in blood and/or fecal matter. In some embodiments, the biomarker is bacterial PGN and the biomarker is detected in fecal matter. In some embodiments, the biomarker is PGLYRP1 and the biomarker is detected in blood and/or fecal matter. In some embodiments, the biomarker is a TREM1 gene activation signature and the biomarker is detected in disease tissue and/or blood. In some embodiments, the antibody agent is characterized in that when administered to the subject it reduces the level and/or activity of TREM1 relative to a comparator. In some embodiments, the comparator comprises an otherwise similar subject not administered the antibody agent, or administered a reference TREM1 inhibitor.

In at least one aspect, the present disclosure provides a method of inhibiting TREM1, the method comprising a step of: contacting a cell, tissue, or subject with a pharmaceutical composition described herein, thereby inhibiting TREM1 in the cell, tissue, or subject. In some embodiments, inhibition of TREM1 comprises a reduction in level and/or activity of TREM1. In some embodiments, inhibition of TREM1 is assessed relative to a comparator.

In at least one aspect, the present disclosure provides a method of antagonizing TREM1-mediated inhibition of maturation of monocytes to macrophages, the method comprising a step of: contacting a cell, tissue, or subject with a pharmaceutical composition described herein, thereby antagonizing TREM1-mediated inhibition of maturation of monocytes to macrophages in the cell, tissue, or subject. In some embodiments, antagonization of TREM1-mediated inhibition of maturation of monocytes to macrophages is assessed relative to a comparator. In some embodiments, the comparator comprises an otherwise similar cell, tissue, or subject not administered the pharmaceutical composition or administered a reference TREM1 inhibitor. In some embodiments, the step of contacting comprises administering the pharmaceutical composition to the cell, tissue, or subject. In some embodiments, the subject is a mammal. In some embodiments, the mammal is a human. In some embodiments, the human is an adult. In some embodiments, the human is a child.

### BRIEF DESCRIPTION OF THE DRAWINGS

FIG. 1A is a t-distributed stochastic neighbor embedding (tSNE) plot showing cell subpopulations identified in single cell transcriptomics data from samples from ID patients. The cell populations were further analyzed for differences across clinical dimensions (e.g., anti-TNFα therapy responder status or inflammation status).

FIG. 1B is a tSNE plot showing the myeloid cell population from FIG. 1A.

FIG. 2 is a tSNE plot of single cell data from inflammatory bowel disease (IBD) patient tissue samples. The cells were shaded based on the hypergeometric odds ratio for the likelihood of a cell's neighborhood being enriched in cells from inflamed samples. The inflammatory monocyte cell population is circled and is highly associated with inflammation.

FIG. 3 is a volcano plot showing the differential abundance of cell types in inflamed samples compared to non-inflamed samples from IBD patients. The x-axis represents the effect size (log 2(fold change)) while the y-axis represents significance (−log₁₀(p-value)). Inflammatory monocytes are the cell type that is most significantly associated with inflamed samples.

FIG. 4A is a tSNE plot of single cell data from inflamed tissues from IBD patients. The cells are shaded based on TREM1 expression levels. Inflammatory monocyte cell population is circled.

FIG. 4B is a tSNE plot of single cell data from non-inflamed tissues from IBD patients. The cells are shaded based on TREM1 expression levels. Inflammatory monocyte cell population is circled.

FIG. 4C is a tSNE plot of single cell data from healthy tissues from non-IBD patients. The cells are shaded based on the TREM1 expression level. Inflammatory monocyte cell population is circled.

FIG. 5A is a tSNE plot of single cell data from peripheral blood mononuclear cells (PBMCs) from IBD patients. The cells are shaded based on TREM1 expression levels.

FIG. 5B is a tSNE plot of single cell data from peripheral blood mononuclear cells (PBMCs) from healthy individuals. The cells are shaded based on TREM1 expression levels.

FIG. 6A is a tSNE plot of single cell data from IBD patient tissue samples. The cells are shaded based on the hypergeometric odds ratio for enrichment in post-treatment samples from non-responders (NR) to anti-TNFα therapy compared with responders (R). Inflammatory monocyte cell population is circled.

FIG. 6B is a tSNE plot of single cell data from post-treatment IBD patient tissue samples from non-responders to anti-TNFα therapy. The cells are shaded based on TREM1 expression levels. Inflammatory monocyte cell population is circled.

FIG. 6C is a tSNE plot of single cell data from pre-treatment and post-treatment tissue samples from Crohn's disease (CD) patients that are non-responders to anti-TNFα therapy. The cells are shaded based on TREM1 expression levels. Inflammatory monocyte cell population is circled.

FIG. 6D is a tSNE plot of single cell data from pre-treatment and post-treatment tissue samples from ulcerative colitis (UC) patients that are non-responders to anti-TNFα therapy. The cells are shaded based on TREM1 expression levels. Inflammatory monocyte cell population is circled.

FIG. 7 shows a correlation between enrichment of TREM1+ myeloid cells (predominantly monocytes), assessed using single-cell transcriptomics on mucosal biopsies, and neutrophil infiltration level, as assessed using the Nancy histopathology index on adjacent biopsies, in inflamed tissues from patients with active ulcerative colitis. Nancy score of <1 denotes that no neutrophil infiltration was observed (‘None’); score of 2 denotes mild neutrophil infiltration (‘Mild’); and score of ≥3 denotes severe neutrophil infiltration (‘Severe’).

FIG. 8A shows binding of anti-TREM1 antibody agent clone A to TREM1 on the cell surface of neutrophils present in whole blood from healthy human subjects as analyzed using flow cytometry. Figure shows data from a single donor that was considered representative of data from 8 different donors.

FIG. 8B shows geometric mean fluorescence intensity (MFI) data of AlexaFluor® 647-labelled anti-TREM1 antibody agent clone A binding on enriched human primary neutrophils (CD45+/CD66abce+) isolated from three healthy volunteer donors. Geometric MFIs of anti-TREM1 antibody agent clone 1 were plotted against anti-TREM1 antibody agent clone 1 concentrations (log scale). Error bars represent mean I SD from technical replicates.

FIG. 8C shows binding of anti-TREM1 antibody agent clone A to TREM1 on the cell surface of total CD14⁺ monocytes present in whole blood from healthy human subjects as analyzed using flow cytometry. Figure shows data from a single donor that was considered representative of data from 8 different donors.

FIG. 8D shows geometric mean fluorescence intensity (MFI) data of AlexaFluor® 647-labelled anti-TREM1 antibody agent clone A binding on enriched human primary monocytes (CD45+/CD14⁺) isolated from four healthy volunteer donors. Geometric MFIs of anti-TREM1 antibody agent clone 1 were plotted against anti-TREM1 antibody agent clone 1 concentrations (log scale). Error bars represent mean±SD from technical replicates.

FIG. 8E shows lack of binding of anti-TREM1 antibody agent clone A to CD45⁺ Lin⁺ (CD3⁺ or CD7⁺ or CD20⁺) cells (e.g., T cells, NK cells, and B cells) present in whole blood from healthy human subjects as analyzed using flow cytometry. Figure shows data from a single donor that was considered representative of data from 8 different donors.

FIG. 9 shows binding of anti-TREM1 antibody agent clone A to cell surface TREM1 on monocytes in response to stimulation with bacterial PGN or LPS, as analyzed using flow cytometry. Figure shows data from a single donor that was considered representative of data from monocytes from 8 different donors.

FIGS. 10A-10F show the media concentration of inflammatory cytokines and chemokines (CCL3 (FIG. 10A), CCL4 (FIG. 10B), IL-1P (FIG. 10C), IL-6 (FIG. 10D), IL-23 (FIG. 10E), and TNFα (FIG. 10F)) secreted by primary monocytes in response to stimulation by PGLYRP1 alone, PGN alone, or a combination of PGN and PGLYRP1. Unstimulated cells (media) are used to show baseline cytokine levels. Mean and standard deviation of 4 replicates is shown. Statistical significance was determined by one-way ANOVA with Tukey's multiple comparison test: ns=not significant; **=p<0.01; ***=p<0.001; ****=p<0.0001. Figures shows data from a single donor that was considered representative of data from 12 different donors.

FIG. 11 shows the media concentration of inflammatory cytokines and chemokines (CCL3, CCL4, and IL-8) secreted by primary neutrophils in response to stimulation by PGLYRP1 alone, PGN alone, or a combination of PGN and PGLYRP1. Unstimulated cells (media) are used to show baseline cytokine levels. Mean and standard deviation of 8 replicates is shown. Statistical significance was determined by one-way ANOVA with Tukey's multiple comparison test: ns=not significant; *=p<0.05; **=p<0.01; ***=p<0.001; ****=p<0.0001. Figure shows data from a single donor that was considered representative of data from 19 different donors.

FIG. 12 shows an amino acid sequence alignment between the extracellular domain (ECD) of human TREM1 (amino acids 20-205 of UniProt accession no.: Q9NP99) and the ECD of human TREM2 (amino acids 19-174 of UniProt accession no.: Q9NZC2).

FIG. 13 shows the maximum bio-layer interferometry (BLI) response in nanometers measured for binding between human TREM2-ECD (BioLegend, 786404 and R&D Systems, 1828-T2-050) and an exemplary anti-TREM1 antibody agent. An anti-TREM2 antibody (R&D Systems, MAB17291) was used as a positive control.

FIG. 14 shows results from a polyspecificity assay. The binding of an anti-TREM1 antibody agent to each of more than 6,000 human membrane proteins expressed on HEK293 cells was measured. Anti-TREM1 antibody agent clone A was highly selective for binding to TREM1.

FIG. 15 shows mean fluorescence intensity (MFI) of G401 cells, which are TMEM178A positive and TREM1 negative, stained with anti-TREM1 antibody agent clone A conjugated to Alexa Fluor 647 (AF647). Geometric mean (GeoMean) MFI of one set of data that was considered representative of three experiments is shown. An isotype control antibody conjugated to AF647 is used as a negative control.

FIG. 16 shows results of a differential scanning fluorimetry assay to evaluate the thermostability of an anti-TREM1 antibody agent.

FIGS. 17A-17F show the media concentration of inflammatory cytokines and chemokines (CCL3 (FIG. 17A), CCL4 (FIG. 17B), IL-1f (FIG. 17C), IL-6 (FIG. 17D), IL-23 (FIG. 17E), and TNFα (FIG. 17F)) secreted by primary monocytes stimulated with a combination of PGN and PGLYRP1, and also treated with anti-TREM1 antibody agent clone A, an isotype control, or not treated with an antibody agent. Unstimulated cells (media) are used to show baseline cytokine levels. Mean and standard deviation of 4 replicates is shown. n.d.=not detected. Statistical significance was determined by one-way ANOVA with Tukey's multiple comparison test: ns=not significant; *=p<0.05; ***=p<0.001; ****=p<0.0001. Figures show data from a single donor that was considered representative of data from 12 different donors.

FIG. 18 shows the media concentration of TNFα secreted by primary human monocytes from healthy volunteers stimulated with PGN, PGLYRP1, or a combination of PGN and PGLYRP1 in the presence of anti-TREM1 antibody agent clone A or an isotype control. A 10-point titration curve for either an anti-TREM1 antibody agent clone A or an isotype control was performed. Unstimulated cells (media) are used to show baseline cytokine levels. Mean and standard deviation of 4 replicates is shown. Figure shows data from a single donor that was considered representative of data from 12 different donors. A nonlinear curve fit of log(antibody) vs. response (three parameters) is shown for the data.

FIGS. 19A-19C show the media concentration of inflammatory cytokines and chemokines (CCL3 (FIG. 19A), CCL4 (FIG. 19B), and IL-8 (FIG. 19C)) secreted by primary neutrophils stimulated with a combination of PGN and PGLYRP1, and also treated with anti-TREM1 antibody agent clone A, an isotype control, or not treated with an antibody agent. Unstimulated cells (media) are used to show baseline cytokine levels. Mean and standard deviation of 8 replicates is shown. n.d.=not detected. Statistical significance was determined by one-way ANOVA with Tukey's multiple comparison test: ns=not significant; ****=p<0.0001. Figures show data from a single donor that was considered representative of data from 17 different donors.

FIG. 20 shows the media concentration of CCL4 secreted by primary human neutrophils from healthy volunteers stimulated with PGN, PGLYRP1, or a combination of PGN and PGLYRP1 in the presence of anti-TREM1 antibody agent clone A or an isotype control. A 10-point titration curve for either an anti-TREM1 antibody agent clone A or an isotype control was performed. Unstimulated cells (media) are used to show baseline cytokine levels. Figure shows data from a single donor that was considered representative of data from 10 different donors. A nonlinear curve fit of log(antibody) vs. response (three parameters) is shown for the data.

FIGS. 21A-21B show results of a transepithelial electrical resistance (TEER) assay in which jejunum epithelial cells from two donors (Jejunum Donor 1 (FIG. 21A) and Jejunum Donor 5 (FIG. 21B)) were treated with differentiation media containing various concentrations of TNFα (15 ng/mL, 30 ng/mL, and 50 ng/mL). Treatment with differentiation media alone (no treatment) or differentiation media containing vehicle (TNFα vehicle) were used as controls. Mean and standard deviation of 3 replicates is shown.

FIGS. 22A-22D show results of a TEER assay in which jejunum epithelial cells from four donors (Donor 1 (FIG. 22A), Donor 2 (FIG. 22B), Donor 5 (FIG. 22C), and Donor 6 (FIG. 22D)) were treated with differentiation media alone (no treatment) or supplement with 50% total volume of either conditioned media from unstimulated human monocytes or from monocytes stimulated with PGN and PGLYRP1. Mean and standard deviation of 3 replicates is shown.

FIGS. 23A-23D show results of a TEER assay in which jejunum epithelial cells from four donors (Donor 1 (FIG. 23A), Donor 2 (FIG. 23B), Donor 5 (FIG. 23C), and Donor 6 (FIG. 23D))were treated with conditioned media produced by cultured monocytes that were unstimulated (conditioned media), stimulated with bacterial peptidoglycan (PGN) and PGLYRP1 (PGN:PGLYPR1), or stimulated with PGN:PGLYPR1 in the presence of anti-TREM1 antibody agent clone A or an isotype control. Cells treated with no conditioned media were also included as a control. Conditioned media was produced from four healthy volunteers (Donors 1, 2, 5, and 6). Conditioned media produced by Donor 1 and 2 monocytes were tested on Jejunum Donor 1 cells, while conditioned media produced by Donor 5 and 6 monocytes were tested on Jejunum Donor 5 cells. Mean and standard deviation of 3 replicates is shown. Statistical significance was determined by one-way ANOVA with Tukey's multiple comparison test: ns=not significant; *=p<0.05; **=p<0.01, ***=p<0.001; ****=p<0.0001.

FIGS. 24A-24B show results of a TEER assay in which epithelial cells were treated with TREM1-conditioned media produced by monocytes that are unstimulated (untreated monocytes), stimulated by bacterial peptidoglycan (PGN) and PGLYRP1 (TREM1-Ligand), or stimulated with TREM1-Ligand in the presence of either an isotype control antibody, anti-TREM1 antibody agent clone A, or an anti-TNFα antibody. FIG. 24A shows results from conditioned media produced by donor monocytes having low TNFα induction (~400 pg/mL) upon stimulation with PGN and PGLYRP1 which responds to anti-TREM1 antibody agent clone A but not to anti-TNFα antibody. FIG. 24B shows results from conditioned media produced by donor monocytes having high TNFα induction (~1000 pg/mL) upon stimulation with PGN and PGLYRP1 which responds to both anti-TREM1 antibody agent clone A and to anti-TNFα antibody.

FIG. 25 shows results from a bio-layer interferometry assay used to evaluate the impact of an anti-TREM1 antibody agent on blocking interaction between TREM1 and its ligand PGLYRP1. Recombinant hTREM1-chFc1 was immobilized on Octet AHC biosensors and then incubated with anti-TREM1 antibody agent clone A or isotype control, followed by incubation with PGLYRP1. Binding of PGLYRP1 to the immobilized TREM1 correlates with measured response. The data was fitted with the sigmoidal (four-parameter) nonlinear regression equation. No fitting curve is provided for isotype control due to lack of a dose-dependent response. The data is from a single experiment that is representative of three replicates.

FIG. 26 shows results from a cell-based assay to evaluate the effect of anti-TREM1 antibody agent clone A on TREM1 cell surface levels on monocytes. Isolated human peripheral CD14⁺ monocytes from healthy volunteers were treated with anti-TREM1 antibody agent clone A or an isotype control antibody. After 18 hours, the cells were stained using a different and non-competitive anti-TREM1 (clone TREM26) antibody conjugated to a fluorescent dye, and total cell surface TREM1 levels (regardless of anti-TREM1 antibody agent clone A binding status) were measured as fluorescence intensity using flow cytometry. Cell background staining is the background fluorescence intensity observed when the cells were stained using an isotype control antibody conjugated to the fluorescent dye. Figure shows data from a single donor that was considered representative of data from 7 different donors.

FIG. 27 shows results from a cell-based assay to evaluate the effect of anti-TREM1 antibody agent clone A on TREM1 cell surface levels on neutrophils. Isolated human peripheral neutrophils from healthy volunteers were treated with anti-TREM1 antibody agent clone A or an isotype control antibody. After 19 hours, the cells were stained using a different and non-competitive anti-TREM1 (clone TREM26) antibody conjugated to a fluorescent dye, and total cell surface TREM1 levels (regardless of anti-TREM1 antibody agent clone A binding status) were measured as fluorescence intensity using flow cytometry. Cell background staining is the background fluorescence intensity observed when the cells were stained using an isotype control antibody conjugated to the fluorescent dye. Figure shows data from a single donor that was considered representative of data from 9 different donors.

FIGS. 28A-28E show flow cytometry results of an assay to evaluate the effect of TREM1 activation and inhibition by an anti-TREM1 antibody on differentiation of inflammatory monocytes to macrophages. Isolated human peripheral monocytes were cultured in the presence of GM-CSF (2.5 ng/mL), IL-23 (10 ng/mL), and IFN-γ (50 ng/mL) for 6 days to generate inflammatory monocytes. Inflammatory monocytes were then cultured in differentiation media containing TGF-β (1 ng/mL) and IL-10 (10 ng/mL) to induce differentiation toward macrophages. In parallel, inflammatory monocytes were cultured in differentiation media containing TGF-β (1 ng/mL) and IL-10 (10 ng/mL) in the presence of PGN (1 μg/mL) and PGLYRP1 (250 nM) to activate TREM1. Anti-TREM1 antibody agent clone A (10 nM) or an isotype control (10 nM) were further added to investigate the effect of TREM1 inhibition on differentiation of inflammatory monocytes to macrophages. After 6 days in the differentiation media, differentiation status was assessed by staining for cell surface markers CD64 and MERTK and determining the percentage of cells in each condition that were inflammatory monocytes (CD64+MERTK−) or macrophages (CD64+MERTK+).FIGS. 28B-28E show results from cell-based assays to characterize the effect of anti-TREM1 antibody agent clone A on TREM-1 pathway-stimulated inflammatory cytokine release from cells derived from inflammatory monocytes. FIGS. 28B-28E show TNFα (FIG. 28B), IL-1β (FIG. 28C), IL-6 (FIG. 28D), and MDC (CCL22; FIG. 28E) production from inflammatory monocytes treated for 6 days with TGFβ and IL-10: in the presence of media alone; peptidoglycan and PGLYRP1 (PGN+PGLYRP1); peptidoglycan, PGLYRP1, and anti-TREM1 antibody agent clone A (PGN+PGLYRP1+Anti-TREM1 Clone A); or peptidoglycan, PLYRP1, and an isotype antibody control (PGN+PGLYRP1+Isotype).

FIGS. 29A-29B show flow cytometry data from a cell-based assay to characterize the potential internalization of membrane TREM1 by anti-TREM1 antibody agent clone A on monocytes. Isolated human monocytes from healthy volunteers were treated with media alone (FIG. 29A) or stimulated with peptidoglycan (PGN; 1 μg/ml; FIG. 29B). Cells were incubated with anti-TREM1 antibody agent clone A or isotype control and stained with anti-hIgG1 Fab-labeled with a pH sensitive dye.

FIG. 30 shows results from a cell-based assay to characterize the effect of anti-TREM1 antibody agent clone A on soluble TREM1 (sTREM1) production levels from primary monocytes.

FIG. 31 shows results from a cell-based assay to characterize the effect of anti-TREM1 antibody agent clone A on TREM1-stimulated TL1a production in primary monocytes.

FIGS. 32A-32C show results from cell-based assays to characterize the effect of anti-TREM1 antibody agent clone A on TREM-1 pathway-stimulated inflammatory cytokine release from cells derived from ulcerative colitis (UC) patient samples. FIGS. 32A-32C show TNFα (FIG. 32A), IL-1β (FIG. 32B), and IL-23 (FIG. 32C) production from cells derived from UC patients treated with: media alone; peptidoglycan and PGLYRP1 (PGN+PGLYRP1); peptidoglycan, PGLYRP1, and anti-TREM1 antibody agent clone A (PGN+PGLYRP1+Anti-TREM1 Clone A); or peptidoglycan, PGLYRP1, and an isotype antibody control (PGN+PGLYRP1+Isotype).

FIGS. 33A-33C show results from cell-based assays to characterize the effect of anti-TREM1 antibody agent clone A on epithelial barrier permeability. Donor-derived epithelial cells plated on transwell plate inserts were cultured until achieving a corrected transepithelial electrical resistance (TEER) measurement about 200 ohms-cm² then treated with: medium alone; TREM1-condition medium+isotype control antibody; TREM1-condition medium+anti-TREM1 antibody agent clone A; or TREM1-condition medium+infliximab (FIG. 33A). FIG. 33B and FIG. 33C are bar graphs showing the relative permeability of jejunum epithelial cell monolayers derived from two representative donors classified as having high TNFα induction (FIG. 33B) or low TNFα induction (FIG. 33C) in the presence of media alone, anti-TREM1 antibody agent clone A, infliximab, or an isotype control antibody.

FIG. 34 shows results from cell-based assays to characterize the effect of anti-TREM1 antibody agent clone A on TREM-1 pathway-stimulated IL-8 release from primary neutrophils derived from two donors compared to an antibody having the binding domain of an anti-TREM1 antibody (mAb 0318, described in WO2019195126A1, incorporated herein by reference) and the same Fc domain as anti-TREM1 antibody agent clone A.

FIG. 35 shows a deuterium exchange heat map of TREM1 protein mixed with Anti-TREM1 antibody agent clone A after 15s, 60s, 180s, 600s, 1800s, and 7200s incubation time.

FIGS. 36A-36C shows bar graphs of inflammatory cytokine/chemokine production of enriched neutrophils from healthy volunteer donors cultured with media alone, 2 mM anti-TREM1 antibody agent clone A, 2 mM isotype control, 50 nM PGLRYP1, or peptidoglycan (PGN; 300 ng/ml). After 24 hours, tissue culture supernatants were harvested and analyzed for CCL3 (FIG. 36A), CCL4 (FIG. 36B), and IL-8 (FIG. 36C) protein quantification. Error bars represent mean±SD from 8 technical replicates. Significance was determined by One-Way ANOVA, Tukey's multiple comparison test; ns=not significant, ****=p<0.0001.

FIGS. 37A-37D shows bar graphs of inflammatory cytokine/chemokine production of enriched primary monocytes from healthy volunteer donors cultured with media alone, 1.25 mM anti-TREM1 antibody agent clone A, 1.25 mM isotype control, 500 nM PGLRYP1, or peptidoglycan (PGN; 100 ng/ml). After 18-24 hours, tissue culture supernatants were harvested and analyzed for CCL3 (FIG. 37A), CCL4 (FIG. 37B), IL-6 (FIG. 37C), and TNFα (FIG. 37D) protein quantification. Error bars represent mean±SD from 4 technical replicates. Significance was determined by One-Way ANOVA, Tukey's multiple comparison test; ns=not significant, **=p<0.01, ***=p<0.001, ****=p<0.0001.

FIG. 38A shows flow cytometry data of percent TREM1 receptor occupancy using saturating concentrations of anti-TREM1 antibody agent clone A. Enriched monocytes (2×10⁵ cells/well) isolated from healthy volunteer donors were activated with peptidoglycan (100 ng/ml) and PGLYRP1 (500 nM) in the presence of a serial dilution of anti-TREM1 antibody agent clone A of isotype control. After 24 hours, TREM1 levels on the surface of monocytes were determined by flow cytometry.

FIG. 38B shows TREM1 receptor occupancy on primary monocytes plotted against normalized TNFα values. TNFα values were normalized to the maximum cytokine released within each donor.

FIGS. 39A-39M shows bar graphs of inflammatory cytokine/chemokine production of whole blood from healthy volunteer donors cultured with 1 pM anti-TREM1 antibody agent clone A, 1 μM isotype control, or 1 μg/ml LPS. After 18-24 hours, tissue culture supernatants were harvested and analyzed for IL-1β (FIG. 39A), IL-2 (FIG. 39B), IL-4 (FIG. 39C), IL-6 (FIG. 39D), IL-8 (FIG. 39E), IL-10 (FIG. 39F), IL-17A (FIG. 39G), IFNγ (FIG. 39H), GM-CSF (FIG. 39I), TNFα (FIG. 39J), CCL2 (FIG. 39K), CCL3 (FIG. 39L), and CCL4 (FIG. 39M) protein quantification. Error bars represent mean±SD from 3 technical replicates. Significance was determined by One-Way ANOVA, Tukey's multiple comparison test; ns=not significant, ***=p<0.001, ****=p<0.0001.

FIGS. 40A-40B show results of flow cytometry analysis of percent of baseline for free TREM1 (FIG. 40A) and total TREM1 (FIG. 40B) on neutrophils with isotype control subtraction following weekly IV administration of anti-TREM1 antibody agent clone A for 14 weeks in cynomolgus monkeys.

FIG. 41 shows a line graph of the percent of baseline (arithmetic means (±SD)) of free TREM1 receptor levels on neutrophils versus time following a single 1-hour IV infusion of various concentrations of anti-TREM1 antibody agent clone A in human subjects. 0 days=the start of infusion.

FIG. 42 shows a line graph of the percent of baseline (arithmetic means (±SD)) of free TREM1 receptor levels on monocytes versus time following a single 1-hour IV infusion of various concentrations of anti-TREM1 antibody agent clone A in human subjects. 0 days=the start of infusion.

FIG. 43 shows a line graph of the percent of baseline (arithmetic means (±SD)) of total TREM1 receptor levels on neutrophils versus time following a single 1-hour IV infusion of various concentrations of anti-TREM1 antibody agent clone A in human subjects. 0 days=the start of infusion.

FIG. 44 shows a line graph of the individual arithmetic means (±SD) of percent TREM1 receptor occupancy on neutrophils versus time following a single 1-hour IV infusion of various concentrations of anti-TREM1 antibody agent clone A in human subjects. 0 days=the start of infusion.

FIG. 45 shows a line graph of the percent of baseline (arithmetic means (±SD)) of total TREM1 receptor levels on monocytes versus time following a single 1-hour IV infusion of various concentrations of anti-TREM1 antibody agent clone A in human subjects. 0 days=the start of infusion.

FIG. 46 shows a line graph of the individual arithmetic means (±SD) of percent TREM1 receptor occupancy on monocytes versus time following a single 1-hour IV infusion of various concentrations of anti-TREM1 antibody agent clone A in human subjects. 0 days=the start of infusion.

FIG. 47 shows a line graph of the individual arithmetic means (±SD) change from baseline of total soluble TREM1 (sTREM1) versus time following a single 1-hour IV infusion of various concentrations of anti-TREM1 antibody agent clone A in human subjects. 0 days=the start of infusion.

FIGS. 48A-48B show stability of anti-TREM1 antibody agent clone A in human (FIG. 48A) and cynomolgus (FIG. 48B) serum as determined by ELISA binding assay.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%1, 19%1, 8%1, 7% 16%, 15%, 14%, 13%1, 2% 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition.

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: As used herein, the term “agent” refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Amino acid: As used herein, the term “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments, it may be used to refer to an amino acid residue of a polypeptide, e.g., an amino acid residue within a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The CDRs of an antibody's antigen-binding site can be determined by the methods described in Lefranc et al., Dev. Comp. Immunol. 27(1):55-77 (2003), Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared to each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, the term “CDR” is a CDR as defined by Lefranc et al., Dev. Comp. Immunol. 27(1):55-77 (2003). In certain embodiments, heavy chain CDRs and light chain CDRs of an antibody are defined using different conventions. For example, in some embodiments, the heavy chain CDRs are defined according to MacCallum (supra), and the light CDRs are defined according to Kabat (supra).

The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of dog, cat, mouse, rabbit, primate, or human antibodies. In some embodiments, an antibody agent may include one or more sequence elements that are human, humanized, primatized, chimeric, etc, as is known in the art. In some embodiments, an antibody agent may include one or more complementarity determining regions that are human and/or one or more constant region sequences that are characteristic of human antibodies. In many embodiments, the term “antibody agent” is used to refer to one or more of the art-known or developed constructs or formats for utilizing antibody structural and functional features in alternative presentation. For example, in some embodiments, an antibody agent utilized in accordance with the present disclosure is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide comprising an antigen binding specificity fused to an Fc; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (e.g., by expression of an engineered nucleic acid). In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antibody polypeptide: As used herein, the term “antibody polypeptide” refers to a polypeptide(s) that includes characteristic sequence element(s) (e.g., one or more CDRs, or a set of CDRs such as each of a CDR1, 2, and 3 as found in reference antibody chain and/or one or more FR regions and/or a set of FR regions, such as, for example, a complete variable region of a heavy or light chain of a reference antibody) of an antibody; in many embodiments, an antibody polypeptide includes sufficient such sequence element(s) that it binds to an epitope (e.g., an epitope bound by a reference antibody including the characteristic sequence element). In some embodiments, an antibody polypeptide is a full-length antibody or heavy or light chain thereof. In some embodiments, an antibody polypeptide is or comprises a complete heavy and/or light chain variable region of a reference antibody; in some such embodiments, an antibody polypeptide includes characteristic antibody sequence element(s) sufficient to confer specific binding to a relevant epitope—i.e., so that the antibody polypeptide includes at least one binding site. In some embodiments, an “antibody polypeptide” may include a binding domain which is homologous or largely homologous (e.g., shows significant sequence homology and/or in some embodiments significant sequence identity) to an immunoglobulin-binding domain. In some embodiments, an antibody polypeptide shows at least 99% identity with an immunoglobulin binding domain. In some embodiments, an “antibody polypeptide” has a binding domain that shows at least 70%, 80%, 85%, 90%, or 95% identity with an immunoglobulin binding domain, for example a reference immunoglobulin binding domain. In some embodiments, an “antibody polypeptide” may have an amino acid sequence identical to that of an antibody, or chain, or variable region thereof (or combination of variable region(s)) that is found in a natural source. In some embodiments, an antibody polypeptide may be prepared by, for example, isolation from a natural source or antibody library, recombinant production in or with a host system, chemical synthesis, etc., or combinations thereof. In some embodiments, an antibody polypeptide is an antibody agent as described herein.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., IMGT, Kabat, Chothia, etc.); those skilled in the art appreciate the differences between and among these systems and are capable of understanding CDR boundaries to the extent required to understand and to practice the claimed invention.

Child. As used herein, the term “child” refers to a human between 1 day and 18 years of age. In some embodiments, a child may be an infant (e.g., may be less than or equal to about 12 months, 11 months, 10 months, 9 months, 8 months, 7 months, 6 months, 5 months, 4 months, 3 months, 2 months or 1 month old); in some embodiments, a child may be older than an infant. In some embodiments, a child may be a toddler (e.g., about 1 to about 3 years old); in some embodiments, a child may be younger than or older than a toddler. In some embodiments, a child may be a teen (e.g., between about 12 and about 18 years old); in some embodiments, a child may be younger than a teen (and/or older or younger than a toddler or older than an infant). Body weight can vary widely across ages and specific children, with a typical range being 4 pounds to 150 pounds.

Combination Therapy: As used herein, the term “combination therapy” refers to a clinical intervention in which a subject is simultaneously exposed to two or more therapeutic regimens (e.g., two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the phrase “corresponding to” refers to a relationship between two entities, events, or phenomena that share sufficient features to be reasonably comparable such that “corresponding” attributes are apparent. For example, in some embodiments, the term may be used in reference to a compound or composition, to designate the position and/or identity of a structural element in the compound or composition through comparison with an appropriate reference compound or composition. For example, in some embodiments, a monomeric residue in a polymer (e.g., an amino acid residue in a polypeptide or a nucleic acid residue in a nucleic acid) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide, in some such embodiments, a domain is characterized by a particular structural element (e.g., a particular amino acid sequence or sequence motif, a-helix character, p-sheet character, coiled-coil character, random coil character, etc.), and/or by a particular functional feature (e.g., binding activity, enzymatic activity, folding activity, signaling activity, etc.).

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Effector cell: as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Framework” or “framework region: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FRI, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FRI, for example, represents the first framework region closest to the amino terminal end of the variable region and 5′ with respect to CDR1, and FRs represents two or more of the sub-regions constituting a framework region.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

High affinity binding: The term “high affinity binding”, as used herein refers to a high degree of tightness with which a particular ligand binds to its partner. Affinities can be measured by any available method, including those known in the art. In some embodiments, binding is considered to be high affinity if the K_{d} is about 500 pM or less (e.g., below about 400 pM, about 300 pM, about 200 pM, about 100 pM, about 90 pM, about 80 pM, about 70 pM, about 60 pM, about 50 pM, about 40 pM, about 30 pM, about 20 pM, about 10 pM, about 5 pM, about 4 pM, about 3 pM, about 2 pM, etc.) in binding assays. In some embodiments, binding is considered to be high affinity if the affinity is stronger (e.g., the K_{d} is lower) for a polypeptide of interest than for a selected reference polypeptide. In some embodiments, binding is considered to be high affinity if the ratio of the Ka for a polypeptide of interest to the Ka for a selected reference polypeptide is 1:1 or less (e.g., 0.9:1, 0.8:1, 0.7:1, 0.6:1, 0.5:1. 0.4:1, 0.3:1, 0.2:1, 0.1:1, 0.05:1, 0.01:1, or less). In some embodiments, binding is considered to be high affinity if the Ka for a polypeptide of interest is about 100% or less (e.g., about 99%, about 98%, about 97%, about 96%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1% or less) of the Ka for a selected reference polypeptide.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improved, increased or reduced: As used herein, the terms “improved,” “increased,” or “reduced,” or grammatically comparable comparative terms thereof, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

K_{D}: as used herein, refers to the dissociation constant of a binding agent (e.g., an antibody or binding component thereof) from a complex with its partner (e.g., the epitope to which the antibody or binding component thereof binds).

Low affinity binding: The term “low affinity binding”, as used herein refers to a low degree of tightness with which a particular ligand binds to its partner. As described herein, affinities can be measured by any available method, including methods known in the art. In some embodiments, binding is considered to be low affinity if the K_{D} is about 501 pM or more (e.g., above about 501 pM, 600 pM, 700 pM, 800 pM, 900 pM, 1 nM, 1.1.nM, 1.2 nM, 1.3 nM, 1.4 nM, 1.5 nM, etc.) In some embodiments, binding is considered to be low affinity if the affinity is the same or lower (e.g., the K_{D} is about the same or higher) for a polypeptide of interest than for a selected reference polypeptide. In some embodiments, binding is considered to be low affinity if the ratio of the K_{D} for a polypeptide of interest to the K_{D} for a selected reference polypeptide is 1:1 or more (e.g., 1.1:1, 1.2:1, 1.3:1, 1.4:1, 1.5:1. 1.6:1, 1.7:1, 1.8:1, 1.9:1, 2:1, 3:1, 4:1, 5:1, 10:1 or more). In some embodiments, binding is considered to be low affinity if the Ka for a polypeptide of interest is 100% or more (e.g., 100%, 105%, 110%, 115%, 120%, 125%, 130%, 135%, 140%, 145%, 150%, 155%, 160%, 165%, 170%, 175%, 180%, 185%, 190%, 195%, 200%, 300%, 400%, 500%, 1000%, or more) of the K_{D} for a selected reference polypeptide.

Non-Responder: As used herein, the term “non-responder” refers to a subject that displays a lack of improvement in clinical signs and symptoms after receiving an anti-inflammatory therapy (e.g., an alternative anti-inflammatory therapy that does not directly target TREM1 (e.g., anti-TNFα therapy)) for a period of time. In some embodiments, a primary non-responder that does not display an improvement in clinical signs and symptoms after receiving an anti-inflammatory therapy. In some embodiments, a secondary non-responder displays an initial improvement in clinical signs or symptoms after receiving an anti-inflammatory therapy but show a statistically significant decrease in such improvement overtime. Those skilled in the art will understand that the medical community may establish an appropriate period of time for any particular disease or condition, or for any particular patient or patient type. To give but a few examples, in some embodiments, the period of time may be at least 8 weeks. In some embodiments, the period of time may be at least 12 weeks. In some embodiments, the period of time may be 14 weeks.

Pharmaceutical composition: As used herein, the term “pharmaceutical composition” refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR, in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | 3- | 1- | | | Hydrophobicity |
|---|---|---|---|---|---|
| Amino Acid | Letter | Letter | Polarity | Charge | (Kyte-Doolittle) |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat”, “treatment”, or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits only early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Therapeutically effective amount: As used herein, the term “therapeutically effective amount” refers to an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder and/or condition. In some embodiments, a therapeutically effective amount is administered in a single dose; in some embodiments, multiple unit doses are required to deliver a therapeutically effective amount.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature.

Vector: As used herein refers to a carrier for a polynucleotide (i.e., a DNA or RNA molecule), which can be used to introduce the polynucleotide into a cell. An “expression vector” is a vector that comprises a sequence encoding a protein and the necessary regulatory regions needed for expression of the sequence in a cell. In some embodiments, the sequence encoding a protein is operably linked to another sequence in the vector. The term “operably linked” means that the regulatory sequences necessary for expression of the sequence encoding a protein are placed in the polynucleotide in the appropriate positions relative to the sequence to effect expression of the protein.

### DETAILED DESCRIPTION

The present disclosure provides, among other things, TREM1 inhibitor agents, which are or include an anti-TREM1 antibody agent. The present disclosure further provides pharmaceutical compositions including such a TREM1 inhibitor agent and methods of using the same for the treatment of a disease, disorder, or condition.

As described herein, the present disclosure further provides an insight that neutrophil levels and/or monocyte levels may be indicative of likely responsiveness to certain anti-inflammatory therapies (e.g., a TREM1 antibody agent described herein). The present disclosure furthermore demonstrates that certain neutrophils (e.g., activated neutrophils) may, in some embodiments, be particularly useful indicators of likely responsiveness to certain anti-inflammatory therapies (e.g., a TREM1 antibody agent described herein). The present disclosure provides various biomarkers that represent and/or act as a proxy for neutrophil levels (e.g., activated neutrophil levels) and/or monocyte levels (e.g., inflammatory monocyte levels), and describes use of such biomarkers in anti-inflammatory therapy (e.g., with a TREM1 antibody agent described herein). In some embodiments, such biomarkers are or comprise a neutrophil product and/or an inflammatory monocyte product.

Among other things, the present disclosure provides an insight that subjects with elevated levels of activated neutrophils and/or an elevated level of inflammatory monocytes may be good candidates for therapy targeting TREM1 (e.g., using a TREM1 antibody agent described herein). Alternatively or additionally, in some embodiments, such subjects may be less good candidates for alternative therapies (e.g., therapy targeting TNFα).

### TREM1

Triggering Receptor Expressed on Myeloid cells 1 (TREM1 or TREM-1) is a member of the Ig-like immunoregulatory receptor family that is found on the surface of certain myeloid cells, specifically neutrophils and subsets of monocytes, that are part of the innate immune system and has been described as an “amplifier” of inflammation. See, for example, Bouchon et al., J. Immunol. 164:4991 (2000); Dantas et al., Intl. Rev. Immunol. 39(4):188-202 (2020).

TABLE 1
TREM1 protein sequences

| | UniProt | | SEQ ID |
|---|---|---|---|
| Organism | Accession No | Sequence | NO: |
| Human | Q9NP99 | MRKTRLWGLLWMLFVSELRAATKLTEEKYE | 1 |
| | | LKEGQTLDVKCDYTLEKFASSQKAWQIIRDG | |
| | | EMPKTLACTERPSKNSHPVQVGRIILEDYHDH | |
| | | GLLRVRMVNLQVEDSGLYQCVIYQPPKEPHM | |
| | | LFDRIRLVVTKGFSGTPGSNENSTQNVYKIPPT | |
| | | TTKALCPLYTSPRTVTQAPPKSTADVSTPDSEI | |
| | | NLTNVTDIIRVPVFNIVILLAGGFLSKSLVFSV | |
| | | LFAVTLRSFVP | |
| Monkey | A0A2K5VHG8 | MRKTRLWGLLWMLFVSELRATTELTEEKYE | 2 |
| (Cynomolgus | | YKEGQTLEVKCDYALEKYANSRKAWQKME | |
| monkey; cyno) | | GKMPKILAKTERPSENSHPVQVGRITLEDYPD | |
| | | HGLLQVRMTNLQVEDSGLYQCVIYQHPKESH | |
| | | VLFNPICLVVTKGSSGTPGSSENSTQNVYRTP | |
| | | STTAKALGPRYTSPRTVTQAPPESTVVVSTPG | |
| | | SEINLTNVTDIIRVPVENIVIIVAGGFLSKSLVF | |
| | | SVLFAVTLRSFGP | |
| Mouse | Q9JKE2 | MRKAGLWGLLCVFFVSEVKAAIVLEEERYDL | 3 |
| | | VEGQTLTVKCPFNIMKYANSQKAWQRLPDG | |
| | | KEPLTLVVTQRPFTRPSEVHMGKFTLKHDPSE | |
| | | AMLQVQMTDLQVTDSGLYRCVIYHPPNDPV | |
| | | VLFHPVRLVVTKGSSDVFTPVIIPITRLTERPILI | |
| | | TTKYSPSDTTTTRSLPKPTAVVSSPGLGVTIIN | |
| | | GTDADSVSTSSVTISVICGLLSKSLVFIILFIVT | |
| | | KRTFG | |
| Dog | E2RP37 | MRKARLWELLWLLFISELQATTEPDEIKYVL | 4 |
| (Canis | | AEGGTLNMKCTTSTWKYTYSQKAWQKLMD | |
| familiaris) | | REKPLTLIFTENVSGDTSQVQRGRYFLEDIPSE | |
| | | AILNVQMTNLQVEDSGLYQCVIYHPQKNPDI | |
| | | LYPRVRLVVTKGITASDKSPTQNLAQISTHPP | |
| | | TTTKAQSTLLASPRTVTQLPPKSTADTSSPDF | |
| | | GVNITNVTNVTSYGFRFSVINIVILVLCGFLSK | |
| | | SLVFTVLIAVTQRSFGP | |
| Cat | M3VZ43 | MKKAGLWGLLWMVFISELQAENESKEEKFIK | 5 |
| (Felis catus) | | EGETLNVNCPFSTEKYSYSQKAWQKLIDGRK | |
| | | PVTLALTTSTSGNPSQVREGRYFLEDIPSEAIL | |
| | | HVQMTNLQVEDSGLYRCVIYYPSKEPDVLSP | |
| | | LFHVVVIKDPLNTTASDENHTRKLQISTFPPTT | |
| | | TRAQGTLVDRPRTVTKLPPKSTADLSSPGFGV | |
| | | NITNVTDVTSYVFRVSVTSIIIIVLCGVLSKSLV | |
| | | FTVLLAVTQRSFGP | |
| Rat | D4ABU7 | MRKAGLWGLLLVFFVSEVKAAIVPEEERYDL | 6 |
| (Rattus | | VEGQTLTVNCPFNIMKYARSRKAWQRLSAG | |
| norvegicus) | | KEPLTLVVTERSSTTSSEVRVGKYTLKDDPTE | |
| | | AMLFVQMTDLQVTDSGLYRCVIYHPPNDPVL | |
| | | LFHPVRLVVTKGSSGVSVPDIIPTTKPTEVPVL | |
| | | ITTKHSTPTRSLPKSTAVVSSPDPGVTINNGTD | |
| | | PTSVSTYNVVVPVVCGLLIKTLIFFVLFVVTK | |
| | | RSFG | |
| Pig | Q6TYI6 | MRSARLGRLLWMLFITEIQAATELPEEKYILA | 44 |
| (Sus scrofa) | | EGETLNVNCPVTVGVYSNSRKAWQKLNRNG | |
| | | KFQTLAITERVSGQVSKVQVGKIFLTDEPSEG | |
| | | MLHVQMTNVQAEDSGLYRCVIYQPPKDPIILF | |
| | | YPVRLVVTNYSSGTPASAETPTQSCSPTTTLPP | |
| | | TTTTNRHRPRPRTVRTVTQFLTDFTTSLSSPGL | |
| | | KVTLTNVTDITRDTEISLILPAVCGLLSKSLVFI | |
| | | VLFVVTRMSFTP | |

The membrane-associated form of TREM1 includes (i) an ectodomain that includes a single Ig V-type domain; (ii) a transmembrane region; and (iii) a cytoplasmic tail that recruits DNAX-Activation Protein 12 (DAP12) for signaling. A soluble form (sTREM1) has also been described, and is thought to be produced by proteolytic cleavage of TREM1 (potentially post-activation; see, for example, Gómez-Piña et al., J. Immunol. 179(6):4065-4073 (2007); Gingras et al., Mol. Immunol. 38:817 (2002); Bostanci et al., J. Dent. Res. 92(2):161-165 (2013); Jolly et al., Cell Mol. Immunol. 18(8):2054-2056 (2021)); or alternative splicing of TREM1 mRNA (see e.g., Baruach et al., J. Immunol 195(12):5725-5731 (2015)). sTREM1 has been reported to compete with TREM1 for binding to at least some ligands. See, Gibot et al., Intensive Care Med. 32:185, 2006. This binding activity may allow sTREM1 to act as a “decoy” receptor for TREM1 ligands, reducing TREM1 activation (See, e.g., WO 2022/258979).

It has also been reported that monomeric TREM1 is inactive; multimerization may be required for activity. See, Carrasco et al., Cell Mol. Immunol. 16:460, 2019.

Various potential ligands have been proposed for TREM1, including PGLYRP1, actin, HMGB1, Hsp70, extracellular cold-inducible RNA-binding protein (eCIRP), and/or one or more infectious-agent-associated antigens (e.g., zymosan, envelope glycoproteins of HIV and/or Marburg virus, egg antigens of Schistosoma mansoni, etc.); see e.g., Fu et al., Front. Immunol. 8:917 (2017) and Singh et al., Expert Opin. Ther. Pat. 31(6):549-561 (2021).

TREM1 has been reported to be up-regulated in response to, for example, hypoxia (in dendritic cells), Vitamin D₃, oxidized low-density lipoprotein, Tumor Necrosis Factor alpha (TNFα), Interleukin 1 beta (IL-1β), GM-CSF, PGE₂, cAMP, and various microbial compounds such as peptidoglycans (PGNs), LPS, and lipoteichoic acid (LTA); see e.g., Rai and Agarwal, Reports 4(2):17 (2021).

Activated TREM1 leads to DAP12 being phosphorylated by Src kinases, triggering a phosphorylation cascade that ultimately results in activation of various transcription factors associated with production of inflammatory mediators (e.g., IL-6, IL-8, IL-1β, and TNFα); see e.g., Dower et al., J. Immunol., 180(5):3520-2534 (2008). TREM1 activation also inhibits pro-apoptotic agents such as BID, BAD, and BAX, and also inhibits mitochondrial release of cytochrome C; see e.g., Campbell et al., mBio, 10(6); e02638-19 (2019); Yuan et al., J. Biol. Chem. 289(21):15118-15129 (2013); and Yuan et al., Am. J. Physiol. Lung Cell. Mol. Physiol., 310(5):L426-38 (2016).

TREM1 expression is down-regulated by PU.1, and by anti-inflammatory cytokines such as IL-10 and TGF-beta; see e.g., Zeng et al., Eur. J. Immunol, 37(8):2300-8 (2007) and Schenk et al., J. Immunol., 174(1):517-24 (2005). Certain other inhibitors of LPS-induced activation have also been reported to decrease TREM1 activity; see e.g., Owens et al., Front. Cell. Neurosci., 11:56 (2017).

### Anti-Inflammatory Therapies

In general, inflammation may be caused by various mechanisms, including, but not limited to, an immune response to injury and infection. Chronic inflammation is a feature of certain diseases (e.g., autoimmune diseases, cancers, inflammatory diseases, etc.) and can result in tissue damage and/or tissue death over time. Anti-inflammatory therapies may inhibit inflammation and/or reduce the symptoms and/or markers of inflammation.

In some embodiments, an anti-inflammatory therapy may reduce the level(s) of one or more inflammatory markers in a subject. In some embodiments, an anti-inflammatory therapy may reduce the level(s) of one or more downstream signaling molecules (e.g., proteins, cytokines, chemokines, etc.) that trigger an inflammatory response. In some embodiments, an anti-inflammatory therapy may bind to a target (e.g., protein, enzyme, etc.) of interest implicated in inflammatory response. In some embodiments, an anti-inflammatory therapy may inhibit function of a target (e.g., protein, enzyme, etc.) of interest implicated in inflammatory response. In some embodiments, an anti-inflammatory therapy may inhibit function of a target (e.g., protein, enzyme, etc.) of interest, preventing inflammatory response. In some embodiments, an anti-inflammatory therapy may inhibit function of a target (e.g., protein, enzyme, etc.) of interest, preventing release/production of downstream signaling molecules (e.g., cytokines, chemokines, etc.) that trigger inflammatory response. In some embodiments, an anti-inflammatory therapy may inhibit the production, processing, secretion, and/or binding of one or more cytokine including, but not limited to, IL-1α, IL-1β, IL-2, IL-6, IL-4, IL-8, IL-10, IL-12, IL-17, IL-22, IL-23, TNFα, GM-CSF, TNF-RII, and IFNγ. In some embodiments, an anti-inflammatory therapy may inhibit the production, processing, secretion, and/or binding of a chemokine including, but not limited to CCL2, CCL3, CCL4, CCL8, CCL19, CCL20, CCL22, CCL24, CXCL1, CXCL5, CXCL9, and CXCL13. In some embodiments, an anti-inflammatory therapy may inhibit the production, processing, secretion, and/or binding of an activator of a B cell, T cell, and/or other cells including, but not limited to IL-1α, IL-1β, IL-6, IL-10, IL-23, APRIL, BAFF, CD30, M-CSF, TNF-RII, and TNFα. In some embodiments, an anti-inflammatory therapy may inhibit the production, processing, secretion, and/or binding of a factor implicated in reducing or impairing epithelial barrier integrity including, but not limited to IL-1α, IL-1, IL-6, IL-8, IL-10, IL-23, GM-CSF, TRAIL, TWEAK, MMP-1, IL-20, TNFR-II, and TNFα. In some embodiments, an anti-inflammatory therapy may prevent or reduce (e.g., inhibit) epithelial barrier damage. In some embodiments, an anti-inflammatory therapy may inhibit the production, processing, secretion, and/or binding of granule proteins including, but not limited to S100A and antimicrobial peptides. In some embodiments, an anti-inflammatory therapy may inhibit the production, processing, secretion, and/or binding of neutrophil products including, but not limited to myeloperoxidase (MPO), elastase, PR3, MMP8, MMP9, prolyl endopeptidase, lipocalin, and Lamp-2; see e.g., Herrero-Cervera et al., Cell Mol Immunol. 19:177-191 (2022).

In many embodiments, anti-inflammatory therapy involves administration of one or more composition(s) that deliver(s) an anti-inflammatory agent (i.e., an agent that inhibits, for example, by reducing the level and/or activity of, a relevant target). Those skilled in the art are aware of various such agents and/or compositions (e.g., as described herein). For completeness, we note that teachings of the present disclosure may be applicable to agent(s) of various chemical classes (e.g., small molecule, nucleic acid, polypeptide, etc.), and those skilled in the art are aware of appropriate modes of delivery for different such classes. For example, in some embodiments, a small molecule agent may be delivered by administration of a composition that includes the agent or that includes a prodrug thereof (e.g., that may be metabolized or otherwise converted to the agent once administered). In some embodiments, a nucleic acid agent may be delivered by administration of a composition that includes the agent, or that includes its complement, or a nucleic acid that otherwise encodes the agent (e.g., so that the agent may be generated in the subject to which the composition is administered). In some embodiments, a polypeptide agent (e.g., a peptide agent, an antibody agent, etc.) may be delivered by administration of a composition that includes the agent, or that includes a precursor to the agent (e.g., so that the agent is generated by processing in the subject to whom the composition is administered) or that includes a nucleic acid that encodes it (e.g., so that the polypeptide agent may be generated in the body to which the composition is administered).

In some embodiments, a target of an anti-inflammatory therapy may be or comprise TREM1. In some embodiments, a target of an anti-inflammatory therapy may be or comprise an alternative target (e.g., TNFα). In some embodiments, anti-inflammatory therapies targeting alternative targets (i.e., targets that are not TREM1), e.g., TNFα inhibitors, may be referred to as alternative therapies (e.g., alternative anti-inflammatory therapies). As described hereinbelow, certain TREM1 inhibitors and inhibitors of alternative targets (e.g., TNFα inhibitors) are known; for some, clinical formulations and protocols have been developed. In some embodiments of the present disclosure, anti-inflammatory therapy is an established therapy, for example administered according to its established protocol (e.g., approved by a relevant regulatory agency such as the United States Food and Drug Administration or the European Medicines Agency). In some embodiments, an anti-inflammatory therapy is or includes an anti-TREM1 antibody agent described herein, a nucleic acid encoding the same, or a composition thereof.

### TREM1 Inhibitors

In some embodiments, an inflammatory disease, disorder, or condition may be treated by inhibition of TREM1, and in particular administration of a TREM1 inhibitor (i.e., by TREM inhibition therapy). In some embodiments, a TREM1 inhibitor is or comprises an anti-TREM1 agent. Various TREM1 inhibitors are known in the art; see e.g., Siskind et al., Front Immunol, 13:907387 (2022), Sigalov, Front Immunol 11:173 (2020); and Brynjolfsson et al., Inflamm Bowel Dis, 22(8):1803-11 (2016).

In some embodiments, a TREM1 inhibitor is or comprises a TREM1 decoy receptor. Without wishing to be bound by any particular theory, TREM1 decoy receptors bind to TREM1 ligands, and thus prevent TREM1 ligand binding to TREM1 and subsequent TREM1 activation. In some embodiments, a TREM1 decoy receptor is or comprises soluble TREM1 (sTREM1) or a TREM1 Fc fusion protein.

In some embodiments, a TREM1 inhibitor is or comprises soluble TREM1 (sTREM1). In some embodiments, sTREM1 is a cleaved extracellular domain of TREM1. Without wishing to be bound by any particular theory, it is thought that sTREM1 acts as a decoy receptor for TREM1 by binding to TREM1 ligands and reducing TREM1 activation and subsequent inflammatory responses (e.g., release of pro-inflammatory cytokines).

In some embodiments, a TREM1 inhibitor is or comprises a TREM1 Fc fusion protein. In some embodiments, a TREM1 Fc fusion protein comprises an extracellular domain of TREM1 (e.g., of mouse TREM1) and an Fc domain (e.g., an Fc portion of human IgG1). Without wishing to be bound by any particular theory, it is thought that TREM1 Fc fusion protein acts (i) as a TREM1 decoy receptor, and (ii) to promote clearance of TREM1 ligands via Fc-receptor mediated endocytosis.

In some embodiments, a TREM1 inhibitor is or comprises a TREM1 decoy peptide. In some embodiments, a TREM1 decoy peptide is a peptide derived from a portion of TREM1 extracellular domain. In some embodiments, a TREM1 decoy peptide is a peptide derived from a portion of a TREM1 ligand. Without wishing to be bound by any particular theory, TREM1 decoy peptides are thought to (i) bind TREM1 and act as a competitive inhibitor that prevents binding of TREM1 ligands to TREM1, and/or (ii) bind TREM1 ligands, and thus prevent TREM1 ligand binding to TREM1 and subsequent TREM1 activation. In some embodiments, a TREM1 decoy peptide is or comprises an LP17 peptide having the amino acid sequence of LQVTDSGLYRCVIYHPP (SEQ ID NO: 7). In some embodiments, a TREM1 decoy peptide is or comprises an M3 peptide having the amino acid sequence of RGFFRGG (SEQ ID NO: 8). In some embodiments, a TREM1 decoy peptide is or comprises an NI peptide having an amino acid sequence of ARNVQHYHMK corresponding to amino acid positions 77-86 of PGLYRP1 (SEQ ID NO: 9).

In some embodiments, a TREM1 inhibitor is or comprises an inhibitor of TREM1 interaction with DAP12. Without wishing to be bound by any particular theory, it is thought that TREM1 mediated signaling occurs via interaction between TREM1 and DAP12. In some embodiments, an inhibitor of TREM1 interaction with DAP12 is or comprises a peptide derived from a portion of TREM1 transmembrane domain. In some embodiments, an inhibitor of TREM1 interaction with DAP12 is or comprises GF9 peptide having the amino acid sequence GLLSKSLVF (SEQ ID NO: 10). In some embodiments, an inhibitor of TREM1 interaction with DAP12 is or comprises a TREM1 sneaking ligand construct (SLC-TREM1). In some embodiments, an SLC-TREM1 comprises an E-selecting targeting domain, a Pseudomonas aeruginosa exotoxin A, and a polypeptide derived from the transmembrane domain of TREM1 having the amino acid sequence LSKSLVF (SEQ ID NO: 11).

In some embodiments, a TREM1 inhibitor is or comprises a peptide derived from TREM1-like transcript 1 (TLT-1). In some embodiments, a peptide derived from TLT-1 is or comprises LR17 peptide having the amino acid sequence LQEEDAGEYGCMVDGAR (SEQ ID NO: 12). In some embodiments, a peptide derived from TLT-1 is or comprises LR12 peptide having the amino acid sequence LQEEDAGEYGCM (SEQ ID NO: 13). In some embodiments, LR12 peptide may be referred to as nangibotide.

### TREM1 Antibody Agent

Disclosed herein are TREM1 antibody agents, which bind (e.g., specifically bind) to TREM1, e.g., with high affinity.

Certain provided TREM1 antibody agents are characterized by an ability to inhibit TREM1 activation. In some embodiments, provided TREM1 antibody agents directly bind and inhibit TREM1. In some embodiments, provided TREM1 antibody agents are characterized by an ability to reduce cell surface expression of TREM1 (e.g., lowered TREM1 expression, cellular internalization of TREM1, clipping of TREM1 to release sTREM1, etc.). In some embodiments, provided TREM1 antibody agents are characterized by an ability to both inhibit TREM1 activation and reduce cell surface expression of TREM1 (e.g., lowered TREM1 expression, cellular internalization of TREM1, clipping of TREM1 to release sTREM1, etc.). Without wishing to be bound by any theory, in some embodiments, provided TREM1 antibody agents are characterized by an ability to both inhibit TREM1 activation and internalize TREM1.

In some embodiments, provided TREM1 antibody agents are characterized by an ability to inhibit TREM1 activation and reduce production of TREM1-driven inflammatory mediators (e.g., cytokines, other cells, chemokines, etc.) by one or more immune cells as compared to a reference (e.g., healthy cells, untreated cells, etc.). In some embodiments, provided TREM1 antibody agents are characterized by an ability to inhibit TREM1 activation and/or reduce production of TREM1-driven inflammatory mediator(s) (e.g., cytokines, chemokines, etc.) from one or more cells (e.g., neutrophils, myeloid cells (including monocytes), etc.) as compared to a reference (e.g., healthy cells, untreated cells, etc.). In some embodiments, provided TREM1 antibody agents are characterized by an ability to inhibit TREM1 activation and/or reduce production of certain inflammatory cytokine(s) (e.g., IL-1β, IL-6, IL-8, IL-23, TNFα, and/or combinations thereof) as compared to a reference (e.g., healthy cells, untreated cells, etc.). In some embodiments, provided TREM1 antibody agents are characterized by an ability to inhibit TREM1 activation and reduce production of certain inflammatory mediators (e.g., APRIL, BAFF, CD30, M-CSF, TRAIL, TWEAK, MMP-1, IL-20, TNFR-II, etc.) as compared to a reference (e.g., healthy cells, untreated cells, etc.). In some embodiments, provided TREM1 antibody agents are characterized by an ability to inhibit TREM1 activation and reduce production of certain inflammatory chemokines (e.g., CCL2, CCL3, CCL4, CCL8, CCL20, CCL22, CCL24, CXCL1, CXCL5, CXCL9, CXCL13, etc.) as compared to a reference (e.g., healthy cells, untreated cells, etc.). In some embodiments, provided TREM1 antibody agents are characterized by an ability to reduce production of multiple pro-inflammatory mediators (e.g., cytokines, chemokines, etc.). In some embodiments, provided TREM1 antibody agents are characterized by an ability to reduce production of multiple pro-inflammatory mediators (e.g., cytokines, chemokines, etc.) while maintaining an innate anti-bacterial response. In some embodiments, provided TREM1 antibody agents are characterized by an ability to reduce production of multiple pro-inflammatory mediators (e.g., cytokines, chemokines, etc.) while preventing further epithelial barrier damage by pro-inflammatory mediators.

In some embodiments, TREM1 antibody agents of the present disclosure are characterized by an ability to interfere with (i.e., reduce or block) binding of TREM1 to one or more of its cognate ligand(s). For example, in some embodiments, a TREM1 antibody agent of the present disclosure interferes (i.e., reduces or blocks) with binding of TREM1 to actin, PGLYRP1, HMGB1, Hsp70, extracellular cold-inducible RNA-binding protein (eCIRP), and/or one or more infectious-agent-associated antigens (e.g., zymosan, envelope glycoproteins of HIV and/or Marburg virus, egg antigens of Schistosoma mansoni, etc.). By interfering with binding of TREM1 with one or more of its ligands, TREM1 antibody agents of the present disclosure are capable of inhibiting downstream TREM1 signaling (e.g., via DAP12) and reduce or prevent immune cell (e.g., neutrophil, monocytes, etc.) activation. In some embodiments, a TREM1 antibody agent of the present disclosure interferes (i.e., reduces or blocks) with binding of TREM1 to PGLYRP1.

In some embodiments, TREM1 antibody agents of the present disclosure are characterized by an ability to reduce expression of TREM1 on the cell surface of immune cells (e.g., neutrophil, monocytes, etc.). In some embodiments, TREM1 antibody agents of the present disclosure are characterized by an ability to promote TREM1 internalization into the immune cell (e.g., neutrophil, monocytes, etc.) upon whose surface TREM1 is expressed. In some embodiments, such an immune cell is a neutrophil or a monocyte. In some embodiments, such an immune cell is a dendritic cell. Internalization of TREM1 can reduce or eliminate downstream TREM1 signaling (e.g., via DAP12), which can further reduce or prevent immune cell (e.g., neutrophil, monocytes, etc.) activation. In some embodiments, TREM1 antibody agents may promote internalization of TREM1 to varying extents and with varying timings and kinetics. Without wishing to be bound by any particular theory, we propose that such variation in ability of TREM1 antibody agents to promote TREM1 internalization may affect one or more pharmacokinetic properties of TREM1 antibody agents.

In some embodiments, TREM1 antibody agents of the present disclosure are characterized by an ability to promote proteolytic cleavage of TREM1. In some embodiments, proteolytic cleavage of TREM1 results in the production of sTREM1. For example, in some embodiments, TREM1 antibody agents of the present disclosure promote the proteolysis of TREM1 by a matrix metalloproteinase (MMP; e.g., MMP1 or MMP9). In promoting the proteolytic cleavage of TREM1, TREM1 antibody agents of the present disclosure can reduce or eliminate downstream TREM1 signaling (e.g., via DAP12), which can further reduce or prevent immune cell (e.g., neutrophil, monocytes, etc.) activation.

In some embodiments, TREM1 antibody agents of the present disclosure are characterized by an ability to inhibit a TREM1 mediated immune response. For example, in some embodiments, a TREM1 antibody agent of the present disclosure inhibits an amplified immune response that is not an innate immune response (e.g., a TLR mediated immune response). In some embodiments, the immune response is mediated by monocytes (e.g., inflammatory monocytes) and/or neutrophils (e.g., activated neutrophils). In some embodiments, the immune response is mediated by dendritic cells.

In some embodiments, provided TREM1 antibody agents are characterized by binding to human TREM1 with a binding affinity (K_{D}) of about 5 nM or less. In some embodiments, provided TREM1 antibody agents are characterized by binding to human TREM1 with a binding affinity (K_{D}) of no more than about 5, 4, 3, 2, 1.5, 1, 0.5, 0.45, 0.4, 0.35, 0.3, 0.25, 0.24, 0.23, 0.22, 0.21, 0.2, 0.19, 0.18, 0.17, 0.16, 0.15, 0.14, 0.13, 0.12, 0.11, 0.1, 0.09, or 0.08 nM. In some embodiments, provided TREM1 antibody agents are characterized by binding to human TREM1 with a binding affinity (K_{D}) of about 0.21 nM. In some embodiments, the human TREM1 is a monomeric human TREM1 extracellular domain (ECD). In some embodiments, binding affinity is measured using a bio-layer interferometry (BLI) assay.

In some embodiments, provided TREM1 antibody agents are characterized by binding to cyno TREM1 with a binding affinity (K_{D}) of about 5 nM or less. In some embodiments, provided TREM1 antibody agents are characterized by binding to cyno TREM1 with a binding affinity (K_{D}) of no more than about 5, 4, 3, 2.5, 2.4, 2.3, 2.2, 2.1, 2, 1.9, 1.8, 1.7, 1.6, 1.5, 1.4, 1.3, 1.2, 1.1, 1, 0.5, 0.45, 0.4, 0.35, 0.3, 0.25, 0.2, 0.15, or 0.1 nM. In some embodiments, provided TREM1 antibody agents are characterized by binding to cyno TREM1 with a binding affinity (K_{D}) of about 1.9 nM. In some embodiments, the cyno TREM1 is a monomeric cyno TREM1 extracellular domain (ECD). In some embodiments, binding affinity is measured using a bio-layer interferometry (BLI) assay.

In some embodiments, provided TREM1 antibody agents are characterized by binding to TREM1 in a cell-binding assay with a binding affinity (K_{D}) of 1 nM or less. In some embodiments, provided TREM1 antibody agents are characterized by binding to TREM1 in a cell-binding assay with a binding affinity (K_{D}) of no more than about 1, 0.9, 0.8 0.7, 0.6, 0.5, 0.45, 0.4, 0.35, 0.3, 0.25, 0.2, 0.1, 0.09, 0.08, 0.07, 0.06, 0.05, 0.04, 0.03, 0.02, or 0.01 nM. In some embodiments, provided TREM1 antibody agents are characterized by binding to TREM1 in a cell-binding assay with a binding affinity (K_{D}) of no more than about 400, 390, 380, 370, 360, 350, 340, 330, 320, 310, 300, 290, 280, 270, 260, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75 pM. In some embodiments, provided TREM1 antibody agents are characterized by binding to TREM1 in a cell-binding assay with a binding affinity (K_{D}) of about 87 pM. In some embodiments, provided TREM1 antibody agents are characterized by binding to TREM1 in a cell-binding assay with a binding affinity (K_{D}) of about 213 pM. In some embodiments, provided TREM1 antibody agents are characterized by binding to TREM1 in a cell-binding assay with a binding affinity (K_{D}) of about 103 pM. In some embodiments, provided TREM1 antibody agents are characterized by binding to TREM1 in a cell-binding assay with a binding affinity (K_{D}) of about 132 pM. In some embodiments, provided TREM1 antibody agents are characterized by binding to TREM1 in a cell-binding assay with a binding affinity (K_{D}) of about 251 pM. In some embodiments, provided TREM1 antibody agents are characterized by binding to TREM1 in a cell-binding assay with a binding affinity (K_{D}) of about 23 pM to about 37 pM. In some embodiments, provided TREM1 antibody agents are characterized by binding to TREM1 in a cell-binding assay with a binding affinity (K_{D}) of about 4 pM to about 13 pM. In some embodiments, a cell binding assay measures binding of a provided TREM1 antibody agent to human TREM1. In some embodiments, a cell binding assay measures binding of a provided TREM1 antibody agent to cyno TREM1. In some embodiments, a cell-binding assay measures binding of a provided TREM1 antibody agent to human TREM1 present on enriched human monocytes. In some embodiments, a cell-binding assay measures binding of a provided TREM1 antibody agent to human TREM1 present on enriched human neutrophils. In some embodiments, a cell-binding assay measures binding of a provided TREM1 antibody agent to human TREM1 present on whole blood human monocytes. In some embodiments, a cell-binding assay measures binding of a provided TREM1 antibody agent to human TREM1 present on whole blood human neutrophils. In some embodiments, a cell-binding assay measures binding of a provided TREM1 antibody agent to cyno TREM1 present on whole blood cyno monocytes. In some embodiments, a cell-binding assay measures binding of a provided TREM1 antibody agent to cyno TREM1 present on whole blood cyno neutrophils.

In some embodiments, provided TREM1 antibody agents are characterized by inhibiting TREM1 activity in a primary cell function assay with a IC50 of about 50 pM, about 48 pM, about 46 pM, about 44 pM, about 42 pM, about 40 pM, about 38 pM, about 36 pM, about 34 pM, about 32 pM, about 30 pM, about 28 pM, about 26 pM, about 24 pM, about 22 pM, about 20 pM, about 18 pM, about 16 pM, about 15 pM, about 14 pM, about 13 pM, about 12 pM, about 11 pM, about 10 pM, about 9 pM, about 8 pM, about 7 pM, about 6 pM, about 5 pM, about 4 pM, about 3 pM, or about 2 pM. In some embodiments, such a primary cell function assay measures inhibition of production of a cytokine or chemokine by an activated neutrophil or monocyte. In some embodiments, a primary cell function assay measures inhibition of CCL3, CCL4, or IL-8 production by activated neutrophils. In some embodiments, inhibition of CCL3, CCL4, or IL-8 production is a reduction of CCL3, CCL4, or IL-8 levels by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to CCL3, CCL4, or IL-8 levels produced by activated neutrophils that are not treated with TREM1 antibody agent. In some embodiments, a primary cell function assay measures inhibition of CCL3, CCL4, TNFα, IL-1β, IL-6, or IL-23 production by monocytes. In some embodiments, inhibition of CCL3, CCL4, TNFα, IL,-10, IL-6, or IL-23 production is a reduction of CCL3, CCL4, TNFα, IL-1β, IL-6, or IL-23 levels by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to CCL3, CCL4, TNFα, IL-1β, IL-6, or IL-23 levels produced by monocytes that are not treated with TREM1 antibody agent. In some embodiments, a primary cell function assay is a human whole blood enriched neutrophil or monocyte primary cell function assay. In some embodiments, a primary cell function assay is a cyno whole blood primary cell function assay.

In some embodiments, provided TREM1 antibody agents are characterized by having a melting temperature that is about 60° C. or more. In some embodiments, provided TREM1 antibody agents are characterized by having a melting temperature that is no less than about 60, about 65, about 66, about 67, about 68, about 69, about 70, about 71, about 72, about 73, about 74, about 75, about 76, about 77, about 78, about 79, about 80, about 85, about 90° C. In some embodiments, provided TREM1 antibody agents are characterized by having a melting temperature that is about 74° C. In some embodiments, melting temperature of an antibody agent is assessed using differential scanning fluorimetry.

In some embodiments, provided TREM1 antibody agents are characterized by an ability to antagonize TREM1-mediated inhibition of maturation of monocytes to macrophages.

In some embodiments, provided TREM1 antibody agents are characterized by an ability to bind non-activated TREM1. In some embodiments, provided TREM1 antibody agents are characterized by an ability to bind activated TREM1 (e.g., TREM1 activated by exposure to bacterial peptidoglycan and PGLYRP1). In some embodiments, provided TREM1 antibody agents are characterized by an ability to bind non-activated TREM1 and activated TREM1. Without wishing to be bound by any particular theory, it is thought that TREM1 activation results in a change in conformation of TREM1. In some embodiments, provided TREM1 antibody agents are characterized by an ability to bind TREM1 in multiple conformational states.

In some embodiments, provided TREM1 antibody agents are characterized in that they do not bind to the surface of cells that do not express TREM1. In some embodiments, such a cell may be a human cell or a cynomolgus monkey cell. Without wishing to be bound by any particular theory, it is thought that cells that do not typically express TREM1 include, but are not limited to, B cell, T cells, and NK cells. In some embodiments, provided TREM1 antibody agents are characterized in that they do not bind to the cell surface of B cells. In some embodiments, provided TREM1 antibody agents are characterized in that they do not bind to the cell surface of T cells. In some embodiments, provided TREM1 antibody agents are characterized in that they do not bind to the cell surface of NK cells.

In some embodiments, provided TREM1 antibody agents are characterized by an ability to inhibit epithelial barrier damage caused by TREM1 and or one or more TREM1 inflammatory mediators. For example, in some embodiments, TREM1 antibody agents of the present disclosure inhibit epithelial barrier damage caused by one or more cytokines amplified by TREM1 (e.g., TNFα, IL-6, IL-8, and IL-1β). In some embodiments, provided TREM1 antibody agents are characterized by an ability to inhibit epithelial barrier damage caused by TREM1 soluble factors associated with epithelial apoptosis (e.g., TWEAK and TRAIL).

In some embodiments, provided TREM1 antibody agents are human antibodies. In some embodiments, provided TREM1 antibody agents are humanized antibodies. In some embodiments, provided TREM1 antibody agents are or comprise human IgG. In some embodiments, provided TREM1 antibody agents are or comprise human IgG1k. In some embodiments, provided TREM1 antibody agents comprise a mutated Fc backbone. In some embodiments, provided TREM1 antibody agents comprise a mutated Fc backbone with reduced binding to Fc receptors. In some embodiments, a TREM1 inhibitor is or comprises an anti-TREM1 chimeric antibody.

In some embodiments, a TREM1 antibody agent disclosed herein competes with a reference TREM1 antibody agent for binding to TREM1. In some embodiments, such a reference TREM1 antibody agent is or comprises an anti-TREM1 antibody described in any of WO2013/120553, WO2016/009086, WO2017/152102, WO2019/195126, WO2020/0163564, WO2021/011681, WO2021/011678, WO2022/233764, WO2022/253991, and WO2022/272018.

In some embodiments, a TREM1 antibody agent of the present disclosure may be an immunoglobulin (e.g., IgG1, IgG2, IgG3, or IgG4), a heterodimer, a Crossmab, a DVD-Ig, a 2-in-1 IgG, an IgG-sc-Fv, an scFv-scFv, a BiTE, a DART, a diabody, a Fab-scFv fusion, a Fab-Fab fusion, or a tandem antibody. In some embodiments, a TREM1 antibody agent of the present disclosure may be an intact IgA, IgG, IgD, IgE, or IgM antibody; an antibody fragment; a single domain antibody; a single chain Fv; or a polypeptide comprising antigen binding specificity fused to an Fc domain.

In some embodiments, a TREM1 antibody agent of the present disclosure comprises a second binding specificity that is to an antigen other than human TREM1. For example, the second binding specificity may be to another pro-inflammatory mediator, or a protein expressed on the surface of an immune cell.

In various embodiments, provided TREM1 antibody agents are characterized by CDR sequences as found in the heavy chain and/or light chain polypeptide variable region sequences included herein as SEQ ID NOs: 14 and 18, respectively. In many embodiments, a provided antibody agent includes all three CDRs from one chain (i.e., heavy chain CDRs 1, 2, and 3, or light chain CDRs 1, 2, and 3, or both). Those skilled in the art will appreciate that different systems for defining CDR sequences have been described such as, for example, the systems described in Lefranc et al., Dev. Comp. Immunol. 27(1):55-77 (2003), Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). Those skilled in the art, moreover, will be readily able to define and utilize those sequences included in SEQ ID NOs: 14 and 18 that make up the CDRs in those sequences.

The present disclosure provides antibody heavy chains and binding portions (e.g., variable domain, or CDRs) thereof, and also provides antibody light chains and binding portions (e.g., variable domain, or CDRs) thereof. In some embodiments, provided heavy chains or binding portions thereof may be utilized together with provided light chains or binding portions thereof. Alternatively or additionally, in some embodiments, provided heavy chains or binding portions thereof may be utilized with different light chains or binding portions thereof, and/or provided light chains or binding portions thereof may be utilized with different heavy chains or binding portions thereof.

In many embodiments, provided antibody heavy chains, or binding portions (e.g., variable domain, or CDRs) thereof are utilized together with provided antibody light chains, or binding portions (e.g., variable domain, or CDRs) thereof. In some embodiments, provided antibody heavy chain and light chain CDRs are incorporated into one or more alternative framework regions.

In some embodiments, provided TREM1 antibody agents of the present disclosure comprise heavy chain CDR1, CDR2, and CDR3 sequences at least 75%, at least 80%, at least 85%, at least 90%, at least 95%, or 100% identical to SEQ ID NOs: 15, 16, and 17, respectively. In some embodiments, provided TREM1 antibody agents of the present disclosure comprise light chain CDR1 and CDR3 sequences at least 75%, at least 80%, at least 85%, at least 90%, at least 95%, or 100% identical to SEQ ID NOs: 19 and 20, respectively, and a light chain CDR2 sequence of GAS. In some embodiments, provided TREM1 antibody agents of the present disclosure comprise heavy chain CDR1, CDR2, and CDR3 sequences 100% identical to SEQ ID NOs: 15, 16, and 17, respectively. In some embodiments, provided TREM1 antibody agents of the present disclosure comprise light chain CDR1 and CDR3 sequences 100% identical to SEQ ID NOs: 19 and 20, respectively, and a light chain CDR2 sequence of GAS.

In some embodiments, provided TREM1 antibody agents comprise a heavy chain variable domain sequence with at least 60% sequence identity to SEQ ID NO: 14, or a portion thereof. In some embodiments, provided TREM1 antibody agents comprise a heavy chain variable domain sequence with at least 70% sequence identity to SEQ ID NO: 14, or a portion thereof. In some embodiments, provided TREM1 antibody agents comprise a heavy chain variable domain sequence with at least 80% sequence identity to SEQ ID NO: 14, or a portion thereof. In some embodiments, provided TREM1 antibody agents comprise a heavy chain variable domain sequence with at least 90% sequence identity to SEQ ID NO: 14, or a portion thereof. In some embodiments, provided TREM1 antibody agents comprise a heavy chain variable domain with at least 95% sequence identity to SEQ ID NO: 14, or a portion thereof. In some embodiments, provided TREM1 antibody agents comprise a heavy chain variable domain sequence with at least 99% sequence identity to SEQ ID NO: 14, or a portion thereof. In some embodiments, provided TREM1 antibody agents comprise a heavy chain variable domain sequence that is identical to SEQ ID NO: 14, or a portion thereof.

In some embodiments, provided TREM1 antibody agents comprise a light chain variable domain sequence with at least 60% sequence identity to SEQ ID NO: 18, or a portion thereof. In some embodiments, provided TREM1 antibody agents comprise a light chain variable domain sequence with at least 70% sequence identity to SEQ ID NO: 18, or a portion thereof. In some embodiments, provided TREM1 antibody agents comprise a light chain variable domain sequence with at least 80% sequence identity to SEQ ID NO: 18, or a portion thereof. In some embodiments, provided TREM1 antibody agents comprise a light chain variable domain sequence with at least 90% sequence identity to SEQ ID NO: 18, or a portion thereof. In some embodiments, provided TREM1 antibody agents comprise a light chain variable domain with at least 95% sequence identity to SEQ ID NO: 18, or a portion thereof. In some embodiments, provided TREM1 antibody agents comprise a light chain variable domain sequence with at least 99% sequence identity to SEQ ID NO: 18, or a portion thereof. In some embodiments, provided TREM1 antibody agents comprise a light chain variable domain sequence that is identical to SEQ ID NO: 18, or a portion thereof.

In some embodiments, provided TREM1 antibody agents comprise a heavy chain variable domain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 14, and a light chain variable domain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 18. In some embodiments, provided TREM1 antibody agents comprise a heavy chain variable domain sequence having 100% sequence identity to SEQ ID NO: 14, and a light chain variable domain sequence having 100% sequence identity to SEQ ID NO: 18.

In some embodiments, provided TREM1 antibody agents are single-chain antibodies (e.g., scFvs) comprising a heavy chain variable domain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 14, and a light chain variable domain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 18. In some embodiments, provided TREM1 antibody agents are scFv antibodies comprising a heavy chain variable domain sequence having 100% sequence identity to SEQ ID NO: 14, and a light chain variable domain sequence having 100% sequence identity to SEQ ID NO: 18.

In some embodiments, a heavy chain and/or light chain of a TREM1 antibody agent of the present disclosure may include not more than 1, 2, 3, 4, or 5 amino acid sequence differences relative to SEQ ID NO: 14 and/or SEQ ID NO: 18, respectively.

In some embodiments, a heavy chain of a TREM1 antibody agent of the present disclosure may include one, two, three, or four framework regions (FR regions) that, collectively, have at least 85%, 90%, 95%, 96%, 97%, 98%, or 99% identity to those found in SEQ ID NO: 14; and/or include not more than 1, 2, 3, 4, or 5 amino acid sequence differences relative to those found in SEQ ID NO: 14. In some embodiments, a heavy chain of a TREM1 antibody agent of the present disclosure may include a variable region (VH) comprising one, two, three, or four FR regions that each independently has an amino acid sequence that is or comprises the amino acid sequence of one of SEQ ID NOs: 21, 22, 23, or 24, an amino acid sequence with at least 85%, 90%, 95%, 96%, 97%, 98%, or 99% identity to one of SEQ ID NOs: 21, 22, 23, or 24, or an amino acid sequence having not more than 1, 2, 3, 4, or 5 amino acid sequence differences relative to one of SEQ ID NOs: 21, 22, 23, or 24.

In some embodiments, a light chain of a TREM1 antibody agent of the present disclosure may include one, two, three, or four framework regions (FR regions) that, collectively, have at least 85%, 90%, 95%, 96%, 97%, 98%, or 99% identity to those found in SEQ ID NO: 18; and/or include not more than 1, 2, 3, 4, or 5 amino acid sequence differences relative to those found in SEQ ID NO: 18.

In some embodiments, a light chain of a TREM1 antibody agent of the present disclosure may include one, two, three, or four framework regions (FR regions) that, collectively, have at least 85%, 90%, 95%, 96%, 97%, 98%, or 99% identity to those found in SEQ ID NO: 18; and/or include not more than 1, 2, 3, 4, or 5 amino acid sequence differences relative to those found in SEQ ID NO: 18. In some embodiments, a light chain of a TREM1 antibody agent of the present disclosure may include a variable region (VL) comprising one, two, three, or four FR regions that each independently has an amino acid sequence that is or comprises the amino acid sequence of one of SEQ ID NOs: 25, 26, 27, or 28, an amino acid sequence with at least 85%, 90%, 95%, 96%, 97%, 98%, or 99% identity to one of SEQ ID NOs: 25, 26, 27, or 28, or an amino acid sequence having not more than 1, 2, 3, 4, or 5 amino acid sequence differences relative to one of SEQ ID NOs: 25, 26, 27, or 28.

In some embodiments, provided TREM1 antibody agents comprise at least one constant region (CH), e.g., a CH1, CH2, and/or CH3 region). In some embodiments, provided TREM1 antibody agents comprise a CH2 region and a CH3 region, e.g., an Fc domain. In some embodiments, an Fc domain is a mouse, a rat, a rabbit, a primate, a human, a dog, a pig, or a cat Fc domain. In some embodiments, provided TREM1 antibody agents comprise an Fc domain selected from an immunoglobulin isotype (e.g., IgA, IgG, IgM, or IgE). In some embodiments, provided TREM1 antibody agents comprise an IgG1, IgG2, IgG2, or IgG4 Fc domain. For example, in some embodiments, provided TREM1 antibody agents of the present disclosure comprise an IgG1 Fc domain. In some embodiments, the IgG1 Fc domain comprises a polypeptide having a sequence with at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO:29, or a portion thereof.

(SEQ ID NO: 29)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK

In some embodiments, an Fc domain is a wild-type Fc domain, e.g., a wild-type human Fc domain. In some embodiments, an Fc domain comprises a variant, e.g., an Fc domain comprising an addition, substitution, or deletion of at least one amino acid residue in an Fc region which results in e.g., reduced or abated affinity for at least on Fc receptor.

The Fc domain of an antibody interacts with a number of receptors or ligands including Fe Receptors (e.g., FcγRI, FcγRIIA, FcγRIIIA), the complement protein C1q, and other molecules such as proteins A and G. These interactions are essential for a variety of effector functions and downstream signaling events including: antibody dependent cell-mediated cytotoxicity (ADCC), antibody-dependent cellular phagocytosis (ADCP), and complement dependent cytotoxicity (CDC).

In some embodiments, provided TREM1 antibody agents comprise a variant Fc domain having one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wild-type Fc region. In some embodiments, a TREM1 antibody agent comprising a variant Fc region has reduced affinity to a human Fc receptor, e.g., FcγRI, FcγRII and/or FcγRIII, or a complement component, e.g., C1q. Exemplary Fe region variants are known in the art.

In some embodiments, provided TREM1 antibody agents comprise a variant IgG1 Fc domain having one or more amino acid additions, substitutions, or deletions. In some embodiments, provided TREM1 antibody agents comprise a IgG1 Fc domain having one or more amino acid substitutions and/or deletions at positions 233, 234, 235 and/or 236 (in accordance with the EU numbering scheme). In some embodiments, provided TREM1 antibody agents comprise an IgG1 Fc domain where the ELLG residues at amino acid positions 233, 234, 235 and 236 (in accordance with the EU numbering scheme) are replaced with PVA (“PVAdelG”). In some embodiments, provided TREM1 antibody agents comprise an IgG1 Fc domain sequence with at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 30, or a portion thereof. In some embodiments, provided TREM1 antibody agents comprise an IgG1 Fc domain sequence identical to SEQ ID NO: 30, or a portion thereof.

(SEQ ID NO: 30)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPPVAGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK

In some embodiments, provided TREM1 antibody agents comprise a heavy chain sequence with at least 60% sequence identity to SEQ ID NO: 31, or a portion thereof. In some embodiments, provided TREM1 antibody agents comprise a heavy chain with at least 70% sequence identity to SEQ ID NO: 31, or a portion thereof. In some embodiments, provided TREM1 antibody agents comprise a heavy chain with at least 80% sequence identity to SEQ ID NO: 31, or a portion thereof. In some embodiments, provided TREM1 antibody agents comprise a heavy chain with at least 90% sequence identity to SEQ ID NO: 31, or a portion thereof. In some embodiments, provided TREM1 antibody agents comprise a heavy chain with at least 95% sequence identity to SEQ ID NO: 31, or a portion thereof. In some embodiments, provided TREM1 antibody agents comprise a heavy chain with at least 99% sequence identity to SEQ ID NO: 31, or a portion thereof. In some embodiments, provided TREM1 antibody agents comprise a heavy chain that is identical to SEQ ID NO: 31, or a portion thereof.

In some embodiments, TREM1 antibody agents comprise a light chain constant region (CL). For example, such a light chain CL may comprise a kappa CL or a lambda CL. In some embodiments, a kappa CL has an amino acid sequence that is or comprises the amino acid sequence of SEQ ID NO: 32, an amino acid sequence at least 85%, 90%, 95%, 96%, 97%, 98%, or 99% identity to SEQ ID NO: 32, or an amino acid sequence having not more than 5, 10, or 20 amino acid sequence differences relative to SEQ ID NO: 32.

(SEQ ID NO: 32)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC

In some embodiments, provided TREM1 antibody agents comprise a light chain sequence with at least 60% sequence identity to SEQ ID NO: 33, or a portion thereof. In some embodiments, provided TREM1 antibody agents comprise a light chain with at least 70% sequence identity to SEQ ID NO: 33, or a portion thereof. In some embodiments, provided TREM1 antibody agents comprise a light chain with at least 80% sequence identity to SEQ ID NO: 33, or a portion thereof. In some embodiments, provided TREM1 antibody agents comprise a light chain with at least 90% sequence identity to SEQ ID NO: 33, or a portion thereof. In some embodiments, provided TREM1 antibody agents comprise a light chain with at least 95% sequence identity to SEQ ID NO: 33, or a portion thereof. In some embodiments, provided TREM1 antibody agents comprise a light chain with at least 99% sequence identity to SEQ ID NO: 33, or a portion thereof. In some embodiments, provided TREM1 antibody agents comprise a light chain that is identical to SEQ ID NO: 33, or a portion thereof.

In some embodiments, provided TREM1 antibody agents comprise a heavy chain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 31, and a light chain sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 33. In some embodiments, provided TREM1 antibody agents comprise a heavy chain sequence having 100% sequence identity to SEQ ID NO: 31, and a light chain sequence having 100% sequence identity to SEQ ID NO: 33.

In some embodiments, provided TREM1 antibody agents comprise two heavy chains, each having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 31, and two light chains each having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 33. In some embodiments, provided TREM1 antibody agents comprise two heavy chains each having a sequence S00% identical to SEQ ID NO. 31, and two light chains each having a sequence 0% identical to SEQ TD NO: 33.

In some embodiments, provided TREM1 antibody agents comprise a heavy chain of SEQ ID NO: 31 and a light chain of SEQ TD NO: 33, or humanized variants thereof. In some embodiments, provided TREM1 antibody agents consist of a heavy chain of SEQ TD NO: 31 and a light chain of SEQ TD NO: 33, or humanized variants thereof.

In some embodiments, a heavy chain and/or light chain of a TREM1 antibody agent of the present disclosure may include not more than 1, 2, 3, 4, or 5 amino acid sequence differences relative to SEQ ID NO: 31 and/or SEQ ID NO: 33, respectively.

Table 2 below provides exemplary TREM1 antibody agent sequences of the present disclosure.

TABLE 2
Exemplary TREM1 antibody agent sequences

| | SEQ ID | | |
|---|---|---|---|
| Clone | NO: | Feature | Sequence |
| A | 14 | VH | EVQLVESGGVLVQPGGSLRLSCAASGITFSNSW |
| | | | MSWVRQAPGKGLEWVANIKQDGSEKYYVDSV |
| | | | KGRFTISRDNAKESLFLQMNSLRAEDTAVYYCA |
| | | | RGGISTFDIWGQGTMVTVSS |
| | 15 | HC CDR1 | GITFSNSW |
| | 16 | HC CDR2 | IKQDGSEK |
| | 17 | HC CDR3 | ARGGISTFDI |
| | 18 | VL | EILMTQSPATLSVSPGERATLSCRASQSVSSNLA |
| | | | WYQQKPGQAPRLLIYGASIRATGIPARFSGSGSR |
| | | | TEFTLTISSLQSEDFAVYYCQQYKNWMYTFGQG |
| | | | TKLEIK |
| | 19 | LC CDR1 | QSVSSN |
| | — | LC CDR2 | GAS |
| | 20 | LC CDR3 | QQYKNWMYT |
| | 21 | HC FR1 | EVQLVESGGVLVQPGGSLRLSCAAS |
| | 22 | HC FR2 | MSWVRQAPGKGLEWVAN |
| | 23 | HC FR3 | YYVDSVKGRFTISRDNAKESLFLQMNSLRAEDT |
| | | | AVYYC |
| | 24 | HC FR4 | WGQGTMVTVSS |
| | 25 | LC FR1 | EILMTQSPATLSVSPGERATLSCRAS |
| | 26 | LC FR2 | LAWYQQKPGQAPRLLIY |
| | 27 | LC FR3 | IRATGIPARFSGSGSRTEFTLTISSLQSEDFAVYY |
| | | | C |
| | 28 | LC FR4 | FGQGTKLEIK |
| | 31 | HC | EVQLVESGGVLVQPGGSLRLSCAASGITFSNSW |
| | | (no signal | MSWVRQAPGKGLEWVANIKQDGSEKYYVDSV |
| | | sequence) | KGRFTISRDNAKESLFLQMNSLRAEDTAVYYCA |
| | | | RGGISTFDIWGQGTMVTVSSASTKGPSVFPLAPS |
| | | | SKSTSGGTAALGCLVKDYFPEPVTVSWNSGALT |
| | | | SGVHTFPAVLQSSGLYSLSSVVTVPSSSLGTQTY |
| | | | ICNVNHKPSNTKVDKKVEPKSCDKTHTCPPCPA |
| | | | PPVAGPSVFLFPPKPKDTLMISRTPEVTCVVVDV |
| | | | SHEDPEVKFNWYVDGVEVHNAKTKPREEQYNS |
| | | | TYRVVSVLTVLHQDWLNGKEYKCKVSNKALPA |
| | | | PIEKTISKAKGQPREPQVYTLPPSRDELTKNQVS |
| | | | LTCLVKGFYPSDIAVEWESNGQPENNYKTTPPV |
| | | | LDSDGSFFLYSKLTVDKSRWQQGNVFSCSVMH |
| | | | EALHNHYTQKSLSLSPGK |
| | 33 | LC | EILMTQSPATLSVSPGERATLSCRASQSVSSNLA |
| | | (no signal | WYQQKPGQAPRLLIYGASIRATGIPARFSGSGSR |
| | | sequence) | TEFTLTISSLQSEDFAVYYCQQYKNWMYTFGQG |
| | | | TKLEIKRTVAAPSVFIFPPSDEQLKSGTASVVCLL |
| | | | NNFYPREAKVQWKVDNALQSGNSQESVTEQDS |
| | | | KDSTYSLSSTLTLSKADYEKHKVYACEVTHQGL |
| | | | SSPVTKSFNRGEC |

### TREM1 Combination Therapy

In some embodiments, an inflammatory disease, disorder, or condition may be treated with a combination of TREM1 antibody agent and another anti-inflammatory therapeutic. For example, in some embodiments, a TREM1 antibody agent may be used for the treatment of an inflammatory disease, disorder, or condition, in combination with one or more of alternative anti-inflammatory therapies that are not targeted to TREM1. Such alternative anti-inflammatory therapies may include, but not limited to, an anti-TFNFα agent, an α₄ integrin-targeted agent (e.g., natalizumab), an α4β7 integrin-targeted agent (e.g. vedolizumab), a Janus kinase (JAK) inhibitor (e.g., tofacitinib), an anti-IL,-12/TL,-23 agent (e.g., ustekinumab), an anti-TL1A agent (e.g., PRA023 and RVT-3101), and a sphingosine 1 phosphate receptor (SIPR) agonist (e.g., ozanimod and etrasimod).

In some embodiments, an inflammatory disease, disorder, or condition may be treated with a combination of TREM1 antibody agent and inhibition of an integrin, and in particular administration of an integrin inhibitor (i.e., by integrin inhibition therapy). In some embodiments, an integrin inhibitor is or comprises an anti-integrin agent. In some embodiments, an anti-integrin agent is or comprises an α₄ integrin-targeted agent (e.g., natalizumab). In some embodiments, an anti-integrin agent is or comprises an α4β7 integrin-targeted agent (e.g. vedolizumab).

In some embodiments, an inflammatory disease, disorder, or condition may be treated with a combination of TREM1 antibody agent and activation of sphingosine 1 phosphate receptor (SIPR), and in particular administration of a SIPR agonist. In some embodiments, a S1PR agonist is or comprises ozanimod. In some embodiments, a S1PR agonist is or comprises etrasimod.

In some embodiments, an inflammatory disease, disorder, or condition may be treated with a combination of TREM1 antibody agent and inhibition of TNFα, and in particular administration of a TNFα inhibitor (i.e., by TNFα inhibition therapy). In some embodiments, a TNFα inhibitor is or comprises an anti-TNFα agent. Examples of anti-TNFα agents approved for use in the United States include monoclonal antibodies such as adalimumab (Humira®), certolizumab pegol (Cimiza®), golimumab (Simponi®), infliximab (Remicade®), and decoy circulating receptor fusion proteins such as etanercept (Enbrel®). These agents are currently approved for use in treatment of indications such as juvenile idiopathic arthritis, psoriatic arthritis, rheumatoid arthritis, ankylosing spondylitis, adult Crohn's disease, pediatric Crohn's disease, ulcerative colitis, plaque psoriasis, hidradenitis suppurativa, and uveitis. In some embodiments, an anti-TNFα agent is a biosimilar of an approved anti-TNFα agent.

In some embodiments, a TNFα inhibitor inhibits TNFα. In some embodiments, a TNF inhibitor specifically and/or selectively inhibits TNFα. In some embodiments, a TNFα inhibitor is or comprises an anti-TNFα antibody agent. In some embodiments, a TNFα inhibitor is or comprises an anti-TNFα antibody. In some embodiments, a TNFα inhibitor is or comprises an anti-TNFα monoclonal antibody. In some embodiments, a TNFα inhibitor is or comprises an anti-TNFα humanized antibody. In some embodiments, a TNFα inhibitor is or comprises an anti-TNFα chimeric antibody.

The present disclosure also provides immunoconjugates comprising a TREM1 antibody agent or portion thereof as described herein, conjugated to an anti-inflammatory agent, a cytotoxic agent (e.g., a chemotherapeutic agent), a toxin (e.g., an enzymatically active toxin of bacterial, fungal, plant, or animal origin, or fragments thereof), and/or a diagnostic agent (e.g., a radioactive isotope, etc.).

### Nucleic Acids

The present disclosure, among other things, provides nucleic acids encoding TREM1 antibody agents described herein, or polypeptides provided herein (e.g., LC polypeptides and/or HC polypeptides). The present disclosure includes polynucleotides encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g. a mammalian cell for production of an antibody agent described herein, or polypeptides provided herein (e.g., LC polypeptides and/or HC polypeptides).

Nucleic acids encoding an antibody agent, or polypeptides provided herein (e.g., LC polypeptides and/or HC polypeptides) may be modified to include codons that are optimized for expression in a particular cell type or organism. Codon optimized sequences are synthetic sequences, and preferably encode an identical polypeptide (or biologically active fragment of a full-length polypeptide which has substantially the same activity as the full-length polypeptide) encoded by a non-codon optimized reference nucleic acid. Codon-optimization may be performed by a commercial antibody development vendor (e.g., Sanyou Bio, Aragen, Viva Biotech, Sino Biological, WuXi Biologics, etc.). In some embodiments, a coding region of a nucleic acids encoding an antibody agent described herein, or polypeptides provided herein (e.g., LC polypeptides and/or HC polypeptides), in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in bacterial cells. Alternatively, the coding sequence may be optimized for expression in a mammalian cell (e.g., a CHO cell). Such a sequence may be described as a codon-optimized sequence.

In some embodiments, the present disclosure provides a single nucleic acid (e.g., DNA or RNA) that encodes a TREM1 antibody agent heavy chain variable domain and a light chain variable domain. In some embodiments, the present disclosure provides a single nucleic acid that encodes a heavy chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 14, and a light chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 18. In some embodiments, a nucleic acid has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 38 and/or SEQ ID NO: 39.

In some embodiments, the present disclosure provides a single nucleic acid (e.g., DNA or RNA) that encodes a TREM1 antibody agent heavy chain and light chain. In some embodiments, the present disclosure provides a single polynucleotide that encodes a heavy chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 31, and a light chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 33. In some embodiments, such a nucleic acid has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 42 and/or SEQ ID NO: 43.

In some embodiments, the present disclosure provides a composition comprising two or more nucleic acids (e.g. DNA or RNA), where one nucleic acid encodes a TREM1 antibody agent heavy chain variable domain, and another nucleic acid encodes a TREM1 antibody agent light chain variable domain. In some embodiments, such a composition comprises a nucleic acid that encodes a heavy chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 14, and another nucleic acid that encodes a light chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 18. In some embodiments, a nucleic acid that encodes a TREM1 antibody agent heavy chain variable domain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 38. In some embodiments, a nucleic acid that encodes a TREM1 antibody agent light chain variable domain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 39.

In some embodiments, the present disclosure provides a composition comprising two or more nucleic acids (e.g. DNA or RNA), where one nucleic acid encodes a TREM1 antibody agent heavy chain, and another nucleic acid encodes a TREM1 antibody agent light chain. In some embodiments, such a composition comprises a nucleic acid that encodes a heavy chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 31, and another nucleic acid that encodes a light chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 33. In some embodiments, a nucleic acid that encodes a TREM1 antibody agent heavy chain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 42. In some embodiments, a nucleic acid that encodes a TREM1 antibody agent heavy chain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 43.

In some embodiments, the present disclosure provides two or more compositions, where one composition comprises a nucleic acid that encodes a TREM1 antibody agent heavy chain variable domain, and another composition comprises a nucleic acid that encodes a TREM1 antibody agent light chain variable domain. In some embodiments, one composition comprises a nucleic acid that encodes a heavy chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 14, and another composition comprises a nucleic acid that encodes a light chain variable domain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 18. In some embodiments, a nucleic acid that encodes a TREM1 antibody agent heavy chain variable domain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 38. In some embodiments, a nucleic acid that encodes a TREM1 antibody agent light chain variable domain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 39.

In some embodiments, the present disclosure provides two or more compositions, where one composition comprises a nucleic acid that encodes a TREM1 antibody agent heavy chain, and another composition comprises a nucleic acid that encodes a TREM1 antibody agent light chain. In some embodiments, one composition comprises a nucleic acid that encodes a heavy chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO:31, and another composition comprises a nucleic acid that encodes a light chain having a sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 33. In some embodiments, a nucleic acid that encodes a TREM1 antibody agent heavy chain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 42. In some embodiments, a nucleic acid that encodes a TREM1 antibody agent heavy chain has a nucleotide sequence that is or comprises a nucleotide sequence that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 43.

Table 3 provides exemplary polynucleotide sequences encoding heavy chains and light chains, or portions thereof, of TREM1 antibody agents of the present disclosure.

TABLE 3
Exemplary nucleic acid sequences encoding TREM1 antibody agents

| | SEQ ID | | |
|---|---|---|---|
| Clone | NO: | Feature | Sequence |
| A | 38 | VH | GAAGTGCAGCTGGTGGAGTCTGGGGGGGTGCTC |
| | | | GTCCAACCCGGCGGCAGCCTCCGACTGAGCTGC |
| | | | GCTGCCTCCGGAATAACTTTCTCCAACTCCTGGA |
| | | | TGAGCTGGGTTCGCCAAGCACCCGGCAAGGGCC |
| | | | TGGAATGGGTGGCCAATATTAAACAGGATGGCT |
| | | | CTGAAAAGTATTATGTGGACTCTGTAAAAGGAA |
| | | | GGTTCACAATTTCACGAGACAATGCCAAGGAAT |
| | | | CCCTGTTCCTGCAAATGAACTCCTTGCGGGCAGA |
| | | | GGATACTGCTGTTTATTACTGTGCTAGGGGAGG |
| | | | AATCAGCACATTCGATATCTGGGGGCAAGGCAC |
| | | | CATGGTCACTGTGTCATCT |
| | 39 | VL | GAGATCCTTATGACCCAATCACCAGCCACCCTG |
| | | | AGTGTCTCCCCCGGAGAACGCGCAACACTGTCC |
| | | | TGTCGGGCCAGCCAGTCCGTATCCTCTAATTTGG |
| | | | CTTGGTATCAGCAAAAGCCAGGACAGGCCCCAC |
| | | | GACTTCTGATCTATGGCGCATCTATAAGGGCAA |
| | | | CAGGAATACCCGCTAGATTTTCTGGGTCAGGGA |
| | | | GCAGAACCGAGTTCACCCTGACCATTTCCAGCCT |
| | | | CCAGTCCGAGGACTTTGCTGTCTACTACTGTCAA |
| | | | CAATATAAGAACTGGATGTATACATTTGGCCAA |
| | | | GGCACCAAGCTGGAGATTAAA |
| | 42 | HC | GAAGTGCAGCTGGTGGAGTCTGGGGGGGTGCTC |
| | | (no signal | GTCCAACCCGGCGGCAGCCTCCGACTGAGCTGC |
| | | sequence) | GCTGCCTCCGGAATAACTTTCTCCAACTCCTGGA |
| | | | TGAGCTGGGTTCGCCAAGCACCCGGCAAGGGCC |
| | | | TGGAATGGGTGGCCAATATTAAACAGGATGGCT |
| | | | CTGAAAAGTATTATGTGGACTCTGTAAAAGGAA |
| | | | GGTTCACAATTTCACGAGACAATGCCAAGGAAT |
| | | | CCCTGTTCCTGCAAATGAACTCCTTGCGGGCAGA |
| | | | GGATACTGCTGTTTATTACTGTGCTAGGGGAGG |
| | | | AATCAGCACATTCGATATCTGGGGGCAAGGCAC |
| | | | CATGGTCACTGTGTCATCTGCTAGCACCAAGGG |
| | | | CCCATCGGTCTTCCCCCTGGCACCCTCCTCCAAG |
| | | | AGCACCTCTGGGGGCACAGCGGCCCTGGGCTGC |
| | | | CTGGTCAAGGACTACTTCCCCGAACCGGTGACG |
| | | | GTGTCGTGGAACTCAGGCGCCCTGACCAGCGGC |
| | | | GTGCACACCTTCCCGGCCGTCCTACAGTCCTCAG |
| | | | GACTCTACTCCCTCAGCAGCGTGGTGACCGTGCC |
| | | | CTCCAGCAGCTTGGGCACCCAGACCTACATCTG |
| | | | CAACGTGAATCACAAGCCCAGCAACACCAAGGT |
| | | | GGACAAGAAGGTTGAGCCCAAATCTTGTGACAA |
| | | | AACTCACACATGCCCACCGTGCCCAGCACCTCC |
| | | | CGTGGCTGGGCCGTCAGTCTTCCTCTTCCCCCCA |
| | | | AAACCCAAGGACACCCTCATGATCTCCCGGACC |
| | | | CCTGAGGTCACATGCGTGGTGGTGGACGTGAGC |
| | | | CACGAAGACCCTGAGGTCAAGTTCAACTGGTAC |
| | | | GTGGACGGCGTGGAGGTGCATAATGCCAAGACA |
| | | | AAGCCGCGGGAGGAGCAGTACAACAGCACGTA |
| | | | CCGTGTGGTCAGCGTCCTCACCGTCCTGCACCAG |
| | | | GACTGGCTGAATGGCAAGGAGTACAAGTGCAAG |
| | | | GTCTCCAACAAAGCCCTCCCAGCCCCCATCGAG |
| | | | AAAACCATCTCCAAAGCCAAAGGGCAGCCCCGA |
| | | | GAACCACAGGTGTACACCCTGCCCCCATCCCGG |
| | | | GACGAGCTGACCAAGAACCAGGTCAGCCTGACC |
| | | | TGCCTGGTCAAAGGCTTCTATCCCAGCGACATCG |
| | | | CCGTGGAGTGGGAGAGCAATGGGCAGCCGGAG |
| | | | AACAACTACAAGACCACGCCTCCCGTGCTGGAC |
| | | | TCCGACGGCTCCTTCTTCCTCTACAGCAAGCTCA |
| | | | CCGTGGACAAGAGCAGGTGGCAGCAGGGGAAC |
| | | | GTCTTCTCATGCTCCGTGATGCATGAGGCTCTGC |
| | | | ACAACCACTACACGCAGAAGAGCCTCTCCCTGT |
| | | | CTCCGGGCAAATAATAG |
| | 43 | LC | GAGATCCTTATGACCCAATCACCAGCCACCCTG |
| | | (no signal | AGTGTCTCCCCCGGAGAACGCGCAACACTGTCC |
| | | sequence) | TGTCGGGCCAGCCAGTCCGTATCCTCTAATTTGG |
| | | | CTTGGTATCAGCAAAAGCCAGGACAGGCCCCAC |
| | | | GACTTCTGATCTATGGCGCATCTATAAGGGCAA |
| | | | CAGGAATACCCGCTAGATTTTCTGGGTCAGGGA |
| | | | GCAGAACCGAGTTCACCCTGACCATTTCCAGCCT |
| | | | CCAGTCCGAGGACTTTGCTGTCTACTACTGTCAA |
| | | | CAATATAAGAACTGGATGTATACATTTGGCCAA |
| | | | GGCACCAAGCTGGAGATTAAACGTACGGTGGCT |
| | | | GCACCATCTGTCTTCATCTTCCCGCCATCTGATG |
| | | | AGCAGTTGAAATCTGGAACTGCCTCTGTTGTGTG |
| | | | CCTGCTGAATAACTTCTATCCCAGAGAGGCCAA |
| | | | AGTACAGTGGAAGGTGGATAACGCCCTCCAATC |
| | | | GGGTAACTCCCAGGAGAGTGTCACAGAGCAGGA |
| | | | CAGCAAGGACAGCACCTACAGCCTCAGCAGCAC |
| | | | CCTGACGCTGAGCAAAGCAGACTACGAGAAACA |
| | | | CAAAGTCTACGCCTGCGAAGTCACCCATCAGGG |
| | | | CCTGAGCTCGCCCGTCACAAAGAGCTTCAACAG |
| | | | GGGAGAGTGTTAATAG |

In some embodiments, the sequence of one or more nucleic acids encoding TREM1 antibody agents of the present disclosure, or portions thereof (e.g., heavy chain, light chain, heavy chain variable domain, or light chain variable domain) is codon optimized for expression in a subject, e.g., humans. The sequence of one or more nucleic acids may also be codon optimized for specific organs.

In brief, codon optimization refers to a process of modifying a nucleotide sequence for enhanced expression in a host cell of interest by replacing at least one codon (e.g., about or more than about 1, 2, 3, 4, 5, 10, 15, 20, 25, 50, 75, 100 or more codons) of the native sequence with codons that are more frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is dependent on, at least in part, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.or.jp/codon/ and these tables can be adapted in a number of ways. See e.g., Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.).

### Compositions

The present disclosure, among other things, provides compositions that comprise or deliver antibody agents, conjugates, combinations, and/or nucleic acids as described herein. In some embodiments, compositions are pharmaceutical compositions comprising one or more antibody agents disclosed herein (e.g., TREM1 antibody agent) and one or more pharmaceutical excipients.

### Polymeric Compositions

Among other things, the present disclosure provides compositions comprising a TREM1 antibody agent or portion thereof (e.g., heavy chain, light chain, heavy chain variable domain, or light chain variable domain) or one or more nucleic acid encoding a TREM1 antibody agent or portion thereof, and one or more polymer(s) to form particulates such as a microsphere, microparticle, nanoparticle, nanosphere, or liposome. Suitable polymers include, but are not limited to, natural or synthetic copolymers or polymers such as gelatin agar, starch, arabinogalactan, albumin, collagen, polyglycolic acid, polylactic aced, glycolide-L(−) lactide poly(epsilon-caprolactone, poly(epsilon-caprolactone-CO-lactic acid), poly(epsilon-caprolactone-CO-glycolic acid), poly(B-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) or poly(methyl methacrylate).

Among other things, the present disclosure provides a composition comprising a microsphere, microparticle, nanoparticle, nanosphere, or liposome encapsulating a nucleic acid encoding a heavy chain variable domain and a light chain variable domain of a TREM1 antibody agent. Among other things, the present disclosure provides a composition comprising a microsphere, microparticle, nanoparticle, nanosphere, or liposome encapsulating a nucleic acid encoding a heavy chain and a light chain of a TREM1 antibody agent.

Among other things, the present disclosure provides a composition comprising a microsphere, microparticle, nanoparticle, nanosphere, or liposome encapsulating at least two nucleic acids where one nucleic acid encodes a heavy chain variable domain of a TREM1 antibody agent and another nucleic acid encodes a light chain variable domain of a TREM1 antibody agent. Among other things, the present disclosure provides a composition comprising a microsphere, microparticle, nanoparticle, nanosphere, or liposome encapsulating at least two nucleic acids where one nucleic acid encodes a heavy chain of a TREM1 antibody agent and another nucleic acid encodes a light chain of a TREM1 antibody agent.

Among other things, the present disclosure provides at least two compositions where one composition comprises a microsphere, microparticle, nanoparticle, nanosphere, or liposome encapsulating a nucleic acid encoding a heavy chain variable domain of a TREM1 antibody agent and another composition comprises a microsphere, microparticle, nanoparticle, nanosphere, or liposome encapsulating a nucleic acid encoding a light chain variable domain of a TREM1 antibody agent. Among other things, the present disclosure provides at least two compositions where one composition comprises a microsphere, microparticle, nanoparticle, nanosphere, or liposome encapsulating a nucleic acid encoding a heavy chain of a TREM1 antibody agent and another composition comprises a microsphere, microparticle, nanoparticle, nanosphere, or liposome encapsulating a nucleic acid encoding a light chain of a TREM1 antibody agent. Among other things, the at least two compositions are delivered to a cell simultaneously. Among other things, the at least two compositions are delivered to a cell at different times.

### Viral Compositions

The present disclosure, among other things, provides compositions for delivering one or more nucleic acids encoding a TREM1 antibody agent or portion thereof (e.g., heavy chain, light chain, heavy chain variable domain, or light chain variable domain) using a viral vector-based platform such as vaccinia, fowlpox, self-replicating alphavirus, marabavirus, adenovirus (see, e.g., Tatsis et al., Adenoviruses, Molecular Therapy (2004) 10, 616-629), a lentivirus, including but not limited to second, third or hybrid second/third generation lentivirus and recombinant lentivirus of any generation designed to target specific cell types or receptors (see, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880), or an adeno-associated virus (“AAV”) vector, as described in more detail in U.S. Pat. No. 5,173,414; Tratschin et al, Mol. Cell. Biol. 5:3251-3260 (1985); Tratschin, et al, Mol. Cell, Biol. 4:2072-2081 (1984); Hermonat and Muzyczka, PNAS 81:64666470 (1984); and Samuiski et al, J. Virol. 63:03822-3828 (1989)). As well understood by persons of skill in the art, to facilitate expression of an antibody agent, or portion thereof, by a cell, viral delivery of one or more nucleic acids encoding the antibody agent or portion thereof may involve genome editing, such as by use of a nuclease-based genome editing system (e.g., a Clustered Regularly Interspaced Short Palindromic Repeats (CRISPR) based genome editing system, a Transcription activator-like effector nuclease (TALEN), a zinc-finger nuclease (ZFN), and a homing endonuclease (HE) based genome editing system or a derivative thereof).

### Pharmaceutical Compositions

The present disclosure, among other things, provides pharmaceutical compositions that comprise or otherwise deliver a TREM1 antibody agent; typically, such pharmaceutical compositions comprise an active agent (e.g., an antibody agent or portion thereof, or a nucleic acid that encodes such antibody agent or portion thereof, etc.) one or more pharmaceutically or physiologically acceptable carriers, diluents, or excipients. Non-limiting examples of, and methods of, preparing such pharmaceutical compositions are well-known in the art, such as, but not limited to, Gennaro, Ed., Remington's Pharmaceutical Sciences, 18th Edition, Mack Publishing Co. (Easton, Pa.) 1990.

When “a therapeutically effective amount” or “an immunologically effective amount” is indicated, a precise amount of a pharmaceutical composition that comprises or delivers a TREM1 antibody agent described herein can be determined by a physician with consideration, for example, of individual differences in age, weight, immune response, and condition of the patient (subject).

In some embodiments, pharmaceutical composition described herein may comprise a pharmaceutically acceptable carrier that is selected for suitability for the mode of administration, solubility, and/or stability of a TREM1 antibody agent, or portion thereof (e.g., heavy chain, light chain, heavy chain variable domain, or light chain variable domain), and/or a nucleic acid encoding a TREM1 antibody agent or portion thereof.

In some embodiments, pharmaceutical compositions described herein may comprise excipients and additives including, but not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients suitable for use in compositions of the present disclosure include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. In some embodiments, an excipient or additive is a polymeric excipient or additive, e.g., including but not limited to, polyvinylpyrrolidones, ficolls (a polymeric sugar), dextrates (e.g., cyclodextrins, such as 2-hydroxypropyl-3-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

In some embodiments, pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a buffer is a salt prepared from an organic acid or base. Exemplary buffers include, but are not limited to, organic acid salts (such as salts of citric acid, ascorbic acid, gluconic acid, carbonic acid, tartaric acid, succinic acid, acetic acid, and phthalic acid), Tris, tromethamine hydrochloride, and phosphate buffers. In some embodiments, a pharmaceutical composition is substantially free of contaminants, e.g., there are no detectable levels of a contaminant (e.g., an endotoxin).

In some embodiments, pharmaceutical compositions described herein may be administered in a manner appropriate to the disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. Typically, pharmaceutical compositions that comprise or deliver antibody agents are injectable or infusible solutions; in some such embodiments, such compositions can be formulated for administration intravenously, subcutaneously, intradermally, intratumorally, intranodally, intramedullary, intramuscularly, transarterially, sublingually, intranasally, topically or intraperitoneally. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety).

In some embodiments, pharmaceutical compositions described herein are administered in combination with (e.g., before, simultaneously, or following) an additional therapy for a symptom, disease or disorder, e.g., a standard-of-care therapy for a symptom, disease or disorder. In some embodiments, pharmaceutical compositions described herein may be administered before or following surgery.

In some embodiments, a dosage of any aforementioned therapy to be administered to a subject will vary with a disease, disorder, or condition being treated and based on a specific subject. Scaling of dosages for human administration can be performed according to art-accepted practices.

### Diseases, Disorders or Conditions

In general, TREM1 antibody agents disclosed herein are useful in any context in which administration of an anti-inflammatory therapy is contemplated or implemented. In some embodiments, TREM1 antibody agents of the present disclosure are useful in the treatment of a subject suffering from a disease, disorder, or condition associated with aberrant (e.g., elevated) inflammation.

Other aspects of the present disclosure relate to an isolated antibody agent that binds to a TREM1 protein as described herein for use in preventing, reducing risk, or treating an individual having a disease, disorder, or injury selected from the group consisting of chronic obstructive pulmonary disease (COPD), acute lung injury or acute respiratory distress syndrome (ARDS), post-stroke injury, post-myocardial infarction injury, ischemic-reperfusion injury, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakoia disease, stroke, acute trauma, chronic trauma, cognitive deficit, memory loss, lupus, acute and chronic colitis, rheumatoid arthritis (RA), atherosclerosis, wound healing, Crohn's disease, inflammatory bowel disease (TBD), ulcerative colitis, obesity, malaria, essential tremor, central nervous system lupus, psoriasis, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, bladder cancer, brain cancer, e.g., glioma such as low-grad glioma, or glioblastoma; breast cancer, cervical cancer, colon cancer, rectal cancer, endometrial cancer, kidney cancer, renal cell cancer, renal pelvis cancer, leukemia, lung cancer, e.g., non-small cell lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, fibrosis, primary or idiopathic myelofibrosis, primary or idiopathic myelosclerosis, myeloid-derived tumors, thyroid cancer, infections, CNS herpes, parasitic infections, Trypanosome infection, Cruzi infection, Pseudomonas aeruginosa infection, Leishmania donovani infection, arthropathy, lyme arthritis, group B Streptococcus infection, Campylobacter jejuni infection, Neisseria meningiditis infection, type I HIV, and Haemophilus influenza. In some embodiments, such an isolated antibody agent is an antibody agent described herein.

### Inflammatory Bowel Disease (IBD)

Inflammatory bowel disease (IBD) is a group of chronic inflammatory conditions affecting the gastrointestinal tract where bacterial dysbiosis and epithelial barrier dysfunction play a key role in disease pathogenesis. In particular, bacterial components have been reported to bind to a selection of receptors, including Toll-Like Receptors (TLRs) and to induce various pathways and mechanisms that can increase intestinal inflammation. IBD can affect other parts of the body (e.g., the joints, skin, bones, eyes, kidneys, and liver) in what is commonly referred to as extraintestinal manifestations (EIMs) of IBD. Anemia is another EM of IBD. IBD often manifests in young adulthood and the relapsing/remitting nature of the condition has been shown to adversely affect subjects' quality of life. Ulcerative colitis (UC) and Crohn's disease (CD) are two of the principal forms of IBD. UC affects the mucosal and submucosal layers of the colon with continuous lesions, where it is common to find crypt abscesses. CD is a transmural disease leading to complications such as fissures, fistulas, strictures, and linear clefts that can result in obstruction of the gut. TREM1 and its associated downstream effects have been implicated in IBD.

The present disclosure provides, among other things, an insight that TREM1-expressing monocytes and neutrophils are enriched in IBD-inflamed gut tissues (e.g., as compared to healthy tissue, IBD non-inflamed tissues, and PBMCs from IBD patients). Bacterial invasion after epithelial barrier dysfunction releases bacterial peptidoglycans (PGNs) into the intestinal mucosa or tissue, leading to activation of TLRs on endogenous neutrophils and myeloid cells (e.g., monocytes). PGN-activated monocytes and neutrophils produce an initial immune response through release of various inflammatory cytokines (e.g., IL-23, TNFα, IL-6, IL-1β, etc.). PGN-activated neutrophils show elevated secretion of PGLYRP1, which, in combination with PGN, can further activate TREM1 on both neutrophils and monocytes and lead to an amplified immune response comprising amplified proinflammatory cytokine and chemokine production, flooding affected tissues with increased levels of inflammatory cytokines and chemokines (e.g., IL-23, TNFα, IK-6, IL-1β, etc.).

Without wishing to be bound by a particular theory, the present disclosure provides an insight that inhibition of TREM1 on neutrophils and myeloid cells (e.g., monocytes) within the gut of IBD patients may preserve innate anti-bacterial response while breaking the chronic cycle of amplified immune response. In some embodiments, such TREM1 inhibitors (e.g., a TREM1 antibody agent disclosed herein) may reduce multiple pro-inflammatory cytokines simultaneously (e.g., IL-23, TNFα, IK-6, IL-1β, etc.) and/or prevent further epithelial barrier damage mediated by cytokines.

There are currently various therapies available for treatment of IBD, including anti-inflammatory aminosalicylates and corticosteroids, immunosuppressive agents, antibiotics, and biologic agents. However, many patients are not fully responsive to conventional treatment and/or treatment may lose efficacy over time. Approved therapies, such as anti-TNFα antibody adalimumab (Humira®), sequester downstream inflammatory cytokines that amplify IBD pathology. However, such therapies are not effective for all patients, with some proving only partially responsive or non-responsive to adalimumab administration.

Without wishing to be bound by a particular theory, current therapies may not adequately address primary sources of amplified inflammatory response (e.g., activation of inflammatory monocytes and/or neutrophils). In particular, it is possible that sequestration of TNFα alone does not limit production and effects of other downstream inflammatory mediators (e.g., cytokines, chemokines, etc.), which lead to additional inflammatory damage, including damage to the epithelial barrier of the gut. There is currently a need to develop therapies that can robustly treat disease across the spectrum of ID and other inflammatory conditions. The present disclosure provides an insight that such therapies can target certain cells and/or cell receptors, which may initiate unwanted inflammatory responses, reducing downstream effects including amplified immune response. In some embodiments, such improved therapies may be applicable to a broader range of subjects and/or provide improved reduction of disease effects/pathology as compared to a reference (e.g., traditional therapies or alternative therapies, such as, e.g., adalimumab). In some embodiments, such improved therapies may inhibit TREM1 and corresponding TREM1-activated inflammatory/immune responses (e.g., preventing production of TREM1-activated inflammatory mediators).

The present disclosure, among other things, provides TREM1 antibody agents, nucleic acids encoding the same, and compositions thereof, for the treatment of IBD. For example, TREM1 antibody agents, nucleic acids encoding the same, and compositions thereof, may be used for, but not limited to, the treatment of Crohn's disease, ulcerative colitis, monogenic and/or very early onset inflammatory bowel disease (VEO-IBD), adult Crohn's disease, pediatric Crohn's disease, post-operative Crohn's disease, ileal fibrostenotic Crohn's disease, and/or immune checkpoint therapy (ICT)-induced Crohn's disease. In some embodiments, treatment of Crohn's disease may include treatment of a complication associated with Crohn's disease, which complication may include, but is not limited to, fibrostenotic disease and/or fistulizing disease.

### Methods of Treatment

Compositions and antibody agents disclosed herein may be used for treatment of diseases associated with cells expressing TREM1 and/or any downstream inflammatory mediators triggered by TREM1 activation. In some embodiments, diseases to be treated may be selected from any disease, disorder, or condition disclosed herein. In some embodiments, diseases to be treated may be inflammatory disorders, e.g., conditions associated with unwanted inflammation or with an increase in inflammation. In some embodiments, a disease to be treated is inflammatory bowel disease (IBD). In some embodiments, a subject to be treated is a mammal (e.g., a human adult or a human child).

In some embodiments, methods of treatment comprise a step of assessing a subject for presence of one or more markers for a disease of interest (e.g., IBD), then administrating a corresponding treatment (e.g., TREM1 antibody agent). In some embodiments, methods of treatment comprise a step of assessing a subject for presence of one or more markers of IBD (e.g., neutrophil density/location, PGLYRP1 levels, calprotectin, sTREM1, etc.), then administrating a corresponding treatment (e.g., TREM1 antibody agent).

In some embodiments, methods of treatment comprise a step of administering a pharmaceutical composition disclosed herein (e.g., TREM1 antibody agent composition). In some embodiments, methods of treatment comprise a step of administering a pharmaceutical composition comprising a TREM1 antibody agent to a subject through an administration route known in the art. In some embodiments, methods of treatment comprise a step of intravenous administration of a pharmaceutical composition comprising a TREM1 antibody agent to a subject. In some embodiments, methods of treatment comprise a step of subcutaneous administration of a pharmaceutical composition comprising a TREM1 antibody agent to a subject.

In some embodiments, provided methods of treatment include treating a subject having an inflammatory disease, disorder or condition, where the subject is determined to have elevated levels of neutrophils (e.g., activated neutrophils) and/or monocytes (e.g., inflammatory monocytes). Typically, such determination is by assessment of a biological sample from the subject. In some embodiments, a biological sample is a blood sample. In some embodiments, a biological sample is a fecal matter sample. In some embodiments, a biological sample is a tissue sample, for example, a sample of a small intestine tissue (e.g., tissue of the duodenum, jejunum, or ileum), large intestine tissue (e.g., cecum, ascending colon, transverse colon, or descending colon), or rectal tissue. In some embodiments, a provided method includes treating the subject with a TREM1 antibody agent and/or composition as disclosed herein.

In some embodiments, provided methods of treatment include treating a subject having an inflammatory disease, disorder or condition, where the subject is determined to have elevated levels of activated neutrophils and/or monocytes (e.g., inflammatory monocytes). For example, in some such embodiments, a subject is determined to have elevated levels of activated neutrophils and/or monocytes by measuring expression of one or more activated neutrophil and/or monocyte biomarker(s) and/or one or more biomarker(s) associated with (i.e., is a proxy for) activated neutrophils and/or monocytes. In some embodiments, a provided method includes treating the subject with a TREM1 antibody agent and/or composition as disclosed herein.

In some embodiments, a subject is determined to have elevated levels of activated neutrophils and/or monocytes by measuring level(s) of one or more activated neutrophil and/or monocyte biomarker(s), and/or one or more biomarker(s) associated with (i.e., is a proxy for) activated neutrophils and/or monocytes, for example as compared to level(s) observed in the subject when the subject does not have the inflammatory disease, disorder or condition, or when the subject does not have an active form of the disease, disorder or condition (e.g., a historical reference). In some embodiments, a subject is determined to have elevated levels of activated neutrophils and/or monocytes by level(s) of one or more activated neutrophil and/or monocyte biomarker(s), and/or one or more biomarker(s) associated with (i.e., is a proxy for) activated neutrophils and/or monocytes, as compared to level(s) observed in an appropriate reference population that is not suffering from the disease, disorder or condition (or is not in an active phase of such disease, disorder or condition); in some such embodiments, an appropriate reference population is the general population.

In some embodiments, provided methods of treatment include treating a subject having an inflammatory disease, disorder or condition, where the subject is predicted to be responsive to treatment with a TREM1 inhibitor and the subject is treated with a TREM1 antibody agent and/or composition as disclosed herein. In some embodiments, a subject is predicted to be responsive to treatment with a TREM1 inhibitor when the subject is determined to have elevated neutrophils and/or monocytes in a biological sample from the subject. In some embodiments, a biological sample is a blood sample. In some embodiments, a biological sample is a fecal matter sample. In some embodiments, a biological sample is a tissue sample, for example a sample of a small intestine tissue (e.g., tissue of the duodenum, jejunum, or ileum), large intestine (e.g., cecum, ascending colon, transverse colon, or descending colon), or rectal tissue. In some embodiments, a subject is predicted to be responsive to treatment with a TREM1 inhibitor when the subject is determined to have elevated levels of activated neutrophils and/or monocytes. For example, in some embodiments, a subject is determined to have elevated levels of activated neutrophils and/or monocytes by measuring the expression of one or more activated neutrophil and/or monocyte biomarker(s), and/or one or more biomarker(s) associated with (i.e., is a proxy for) activated neutrophils and/or monocytes.

In some embodiments, a subject is predicted to be responsive to treatment with a TREM1 inhibitor when the subject is determined to have elevated levels of activated neutrophils and/or monocytes by measuring elevated levels of one or more activated neutrophil and/or monocyte biomarker(s), and/or one or more biomarker(s) associated with (i.e., is a proxy for) activated neutrophils and/or monocytes, as compared to levels observed in the subject when the subject does not have the inflammatory disease, disorder or condition, or when the subject does not have an active form of the disease, disorder or condition (e.g., a historical reference for the subject).

In some embodiments, a subject is predicted to be responsive to treatment with a TREM1 inhibitor when the subject is determined to have elevated levels of activated neutrophils and/or monocytes by measuring elevated levels of one or more activated neutrophil and/or monocyte biomarker(s), and/or one or more biomarker(s) associated with (i.e., is a proxy for) activated neutrophils and/or monocytes, as compared to levels observed in an appropriate reference population that is not suffering from the disease, disorder or condition (or is not in an active phase of such disease, disorder or condition); in some such embodiments, an appropriate reference population is the general population.

In some embodiments, provided methods of treatment include treating a subject having an inflammatory disease, disorder or condition, with a TREM1 antibody agent and/or composition as disclosed herein, based on measured levels of one or more activated neutrophil and/or monocyte biomarker(s), and/or one or more biomarker(s) associated with (i.e., is a proxy for) activated neutrophils and/or monocytes, in the subject. In some embodiments, such a method includes treating a subject with a TREM1 antibody agent and/or composition as disclosed herein when such measured levels, is/are elevated as compared to those observed in the subject when the subject does not have the inflammatory disease, disorder or condition, or when the subject does not have an active form of the disease, disorder or condition. In some embodiments, such a method includes treating a subject with a TREM1 antibody agent and/or composition as disclosed herein when determined level(s) of one or more activated neutrophil and/or monocyte biomarker(s), and/or one or more biomarker(s) associated with activated neutrophils and/or monocytes, is/are elevated as compared to level(s) observed in an appropriate reference population that is not suffering from the disease, disorder or condition (or is not in an active phase of such disease, disorder or condition); in some such embodiments, an appropriate reference population is the general population.

In some embodiments, provided methods of treatment include treating a subject having an inflammatory disease, disorder, or condition who is resistant and/or non-responsive to one or more alternative anti-inflammatory therapies that do not directly target TREM1 (e.g., TNFα inhibitor therapy). In some embodiments, an alternative anti-inflammatory therapy is or comprises anti-inflammatory aminosalicylates and corticosteroids, immunosuppressive agents, antibiotics, and biologic agents (e.g., biologic agents that do not target TREM1). In some embodiments, an alternative anti-inflammatory therapy is or comprises an TNFα inhibitor, a Janus kinase (JAK) inhibitor (e.g., upadacitinib, tofacitinib and filgotinib), an anti-integrin therapy (e.g., an α₄ integrin-targeted agent (e.g., natalizumab) or an α4β7 integrin-targeted agent (e.g. vedolizumab)), an anti-IL23 therapy (e.g., guselkumab, mirikizumab, and brazikumab); an anti-IL-12/23 therapy (e.g., ustekinumab); an anti-IL-23A therapy (e.g., risankizumab); an S1PR agonist or modulator therapy (e.g., ozanimod and etrasimod); a 5-aminosalicylate therapy (e.g., mesalamine, olsalazine, balsalazide, and sulfasalazine); an immunomodulatory therapy (e.g., azathioprine, 6-mercaptopurine, and Methotrexate); a corticosteroid therapy (e.g., prednisone, methylprednisolone, hydrocortisone, and budesonide); an anti-TL1 A therapy (e.g., PRA023 and RVT-3101); a kinase inhibitor (e.g., ritlecitinib), a TYK2 inhibitor therapy (e.g., deucravacitinib), an anti-IL-36 therapy (e.g., spesolimab); an anti-IL-13 therapy (e.g., dupilumab), a miR-124 upregulator therapy (e.g., obefazimod), a TLR9 agonist therapy (e.g., cobitolimod); or a combination thereof. In some embodiments, TNFα inhibitor therapy involves treatment with an anti-TNFα antibody agent. In some embodiments, TNFα inhibitor therapy includes treatment with adalimumab (Humira®), certolizumab pegol (Cimiza®), golimumab (Simponi®), infliximab (Remicade®), or decoy circulating receptor fusion proteins such as etanercept (Enbrel®), or a biosimilar thereof.

In some embodiments, provided methods of treatment include treating a subject having an inflammatory disease, disorder, or condition, wherein the subject is predicted to be resistant and/or non-responsive to TNFα inhibitor therapy. In some embodiments, a provided method includes treating with a TREM1 antibody agent and/or composition as disclosed herein. In some embodiments, TNFα inhibitor therapy involves treatment with an anti-TNFα antibody agent (e.g., an anti-TNFα monoclonal antibody, an anti-TNFα humanized antibody, and/or an anti-TNFα chimeric antibody). In some embodiments, TNFα inhibitor therapy involves treatment with a decoy circulating receptor fusion protein. In some embodiments, TNFα inhibitor therapy includes treatment with adalimumab, certolizumab, golimumab, infliximab, and/or etanercept. In some embodiments, a TNFα inhibitor is or comprises a biosimilar of a TNFα inhibitor. In some embodiments, a subject is predicted to be resistant and/or non-responsive to TNFα inhibitor therapy when the subject is determined to have elevated levels of neutrophils (e.g., activated neutrophils), e.g., by assessment of a biological sample from the subject. In some embodiments, a subject is predicted to be resistant and/or non-responsive to TNFα inhibitor therapy when the subject is determined to have elevated levels of neutrophils (e.g., activated neutrophils) and/or monocytes (e.g., inflammatory monocytes), e.g., by assessment of a biological sample from the subject. In some embodiments, a biological sample is a blood sample. In some embodiments, a biological sample is a fecal matter sample. In some embodiments, a biological sample is a tissue sample, for example a sample of a small intestine tissue (e.g., tissue of the duodenum, jejunum, or ileum), large intestine (e.g., cecum, ascending colon, transverse colon, or descending colon), or rectal tissue.

In some embodiments, a subject is predicted to be resistant and/or non-responsive to one or more alternative anti-inflammatory therapies that do not directly target TREM1 (e.g., TNFα inhibitor therapy) when the subject is determined to have elevated levels of activated neutrophils and/or monocytes. For example, in some embodiments, a subject is determined to have elevated levels of activated neutrophils and/or monocytes by measuring the expression of one or more activated neutrophil and/or monocyte biomarker(s) and/or one or more biomarker(s) associated with (i.e., is a proxy for) activated neutrophils and/or monocytes. In some embodiments, a subject is predicted to be resistant and/or non-responsive to one or more alternative anti-inflammatory therapies that do not directly target TREM1 (e.g., TNFα inhibitor therapy) when the subject is determined to have elevated levels of one or more activated neutrophil and/or monocyte biomarker(s), and/or one or more biomarker(s) associated with (i.e. is a proxy for) activated neutrophils and/or monocytes, for example as compared to that observed when the subject does not have the inflammatory disease, disorder or condition, or when the subject does not have an active form of the disease, disorder or condition (e.g., a historical reference for the subject). In some embodiments, a subject is determined to be resistant and/or non-responsive to one or more alternative anti-inflammatory therapies that do not directly target TREM1 (e.g., TNFα inhibitor therapy) when the subject is determined to have elevated levels relative to level(s) observed in an appropriate reference population that is not suffering from the disease, disorder or condition (or is not in an active phase of such disease, disorder or condition); in some such embodiments, an appropriate reference population is the general population.

In some embodiments, methods provided by the present disclosure include methods of identifying or characterizing subject(s) having an inflammatory disease, disorder or condition, and determining those likely to be responsive (e.g., predicted to be responsive) to treatment with a TREM1 antibody agent and/or composition as disclosed herein. In some embodiments, a subject is predicted to be responsive to treatment when the subject is determined to have elevated neutrophil and/or monocyte levels in a biological sample from the subject. In some embodiments, a biological sample is a blood sample. In some embodiments, a biological sample is a fecal matter sample. In some embodiments, a biological sample is a tissue sample, for example a sample of a small intestine tissue (e.g., tissue of the duodenum, jejunum, or ileum), large intestine (e.g., cecum, ascending colon, transverse colon, or descending colon), or rectal tissue. In some embodiments, a subject is predicted to be responsive to treatment when the subject is determined to have elevated levels of activated neutrophils and/or monocytes. In some embodiments, a subject is predicted to be responsive to treatment when the subject is determined to have elevated levels of one or more activated neutrophil and/or monocyte biomarker(s), and/or one or more biomarker(s) associated with (i.e. is a proxy for) activated neutrophils and/or monocytes, for example as compared to that observed when the subject does not have the inflammatory disease, disorder or condition, or when the subject does not have an active form of the disease, disorder or condition (e.g., a historical reference for the subject). In some embodiments, a subject is predicted to be responsive to treatment when the subject is determined to have elevated levels relative to level(s) observed in an appropriate reference population that is not suffering from the disease, disorder or condition (or is not in an active phase of such disease, disorder or condition); in some such embodiments, an appropriate reference population is the general population.

In some embodiments, methods provided by the present disclosure include methods for monitoring the efficacy of treatment in a subject by a TREM1 antibody agent and/or composition as disclosed herein. In some embodiments, efficacy in a subject is monitored by measuring the expression of one or more activated neutrophil and/or monocyte biomarker(s), and/or one or more biomarker(s) associated with (i.e. is a proxy for) activated neutrophils and/or monocytes. In some embodiments, efficacy in a subject is monitored by measuring the levels of one or more activated neutrophil and/or monocyte biomarker(s), and/or one or more biomarker(s) associated with (i.e. is a proxy for) activated neutrophils and/or monocytes in the subject, for example as compared to that observed when the subject does not have the inflammatory disease, disorder or condition, or when the subject does not have an active form of the disease, disorder or condition (e.g., a historical reference for the subject). In some embodiments, efficacy in a subject is monitored by measuring the levels of one or more activated neutrophil and/or monocyte biomarker(s), and/or one or more biomarker(s) associated with (i.e. is a proxy for) activated neutrophils and/or monocytes in the subject, for example as compared to an appropriate reference population that is not suffering from the disease, disorder, or condition (or is not in an active phase of such disease, disordered, or condition); in some such embodiments, an appropriate reference population is the general population. In some embodiments, provided methods include modifying the amount of anti-inflammatory therapy (e.g., a TREM1 antibody agent and/or composition as disclosed herein) administered to a subject based on expression or expression level of one or more activated neutrophil and/or monocyte biomarker(s), and/or one or more biomarker(s) associated with (i.e., is a proxy for) activated neutrophils and/or monocytes. In some embodiments, provided methods include modifying administration of anti-inflammatory therapy (e.g., a TREM1 antibody agent and/or composition as disclosed herein) to target a specific tissue that expresses, or expresses elevated levels of one or more activated neutrophil and/or monocyte biomarker(s), and/or one or more biomarker(s) associated with (i.e. is a proxy for) activated neutrophils and/or monocytes. For example, in some such embodiments, anti-inflammatory therapy may be modified to specifically target small intestine tissue (e.g., tissue of the duodenum, jejunum, or ileum), large intestine (e.g., cecum, ascending colon, transverse colon, or descending colon), and/or rectal tissue expressing, or expressing elevated levels of, one or more activated neutrophil and/or monocyte biomarker(s), and/or one or more biomarker(s) associated with (i.e. is a proxy for) activated neutrophils and/or monocytes.

In some embodiments of provided methods of treatment, the one or more biomarker(s) and/or one or more biomarker(s) associated with (i.e., is a proxy for) activated neutrophils and/or monocytes is a cell-based biomarker. For example, in some embodiments, the cell-based biomarker is a blood and/or tissue neutrophil count, monocyte count, or neutrophil and monocyte count. In some embodiments, the one or more activated neutrophil and/or monocyte biomarker and/or one or more biomarker(s) associated with (i.e., is a proxy for) activated neutrophils and/or monocytes is TREM1 RNA in monocytes and/or neutrophils. In some embodiments, the one or more activated neutrophil and/or monocyte biomarker(s) and/or one or more biomarker(s) associated with (i.e., is a proxy for) activated neutrophils and/or monocytes is TREM1 on the cell surface of monocytes and/or neutrophils.

In some embodiments of provided methods of treatment, the one or more activated neutrophil and/or monocyte biomarker(s) and/or one or more biomarker(s) associated with (i.e., is a proxy for) activated neutrophils and/or monocytes is a tissue biomarker. For example, in some embodiments, the tissue biomarker is mucosal ulceration. In some embodiments, the one or more activated neutrophil biomarker(s) and/or one or more biomarker(s) associated with (i.e., is a proxy for) activated neutrophils is the presence of neutrophils in the lamina propria or neutrophils in an epithelial layer of the subject. In some embodiments, the one or more activated neutrophil biomarker(s) and/or one or more biomarker(s) associated with (i.e., is a proxy for) activated neutrophils is the presence of lymphocytes, plasmocytes and/or eosinophils in the lamina propria of the subject.

In some embodiments of provided methods of treatment, the one or more activated neutrophil biomarker(s) and/or one or more biomarker(s) associated with (i.e., is a proxy for) activated neutrophils is a product of neutrophils. For example, in some embodiments, the product of neutrophils is calprotectin, PGLYRP1, or soluble TREM1.

In some embodiments of provided methods of treatment, the one or more monocyte biomarker(s) and/or one or more biomarker(s) associated with (i.e., is a proxy for) monocytes is a product of monocytes. For example, in some embodiments, the product of monocytes is soluble TREM1.

In some embodiments of provided methods of treatment, the one or more activated neutrophil and/or monocyte biomarker(s) and/or one or more biomarker associated with (i.e., is a proxy for) activated neutrophils and/or monocytes is a TREM1 ligand. For example, in some embodiments, the TREM1 ligand is bacterial peptidoglycan (PGN) and/or PGLYRP1.

In some embodiments of provided methods of treatment, the one or more activated neutrophil and/or monocytes biomarker(s) and/or one or more biomarker(s) associated with (i.e., is a proxy for) activated neutrophils and/or monocytes is a TREM1 gene activation signature.

In some embodiments of provided methods of treatment, the one or more activated neutrophil and/or monocyte biomarker(s) and/or one or more biomarker(s) associated with (i.e. is a proxy for) activated neutrophils and/or monocytes includes any one or combination of: a neutrophil and/or monocyte count detected in diseased tissue; TREM1 on the cell surface of monocytes and/or neutrophils in diseased tissue; TREM1 RNA in monocytes and/or neutrophils in diseased tissue; a tissue biomarker in diseased tissue; soluble TREM1 in a blood and/or fecal sample; calprotectin in a blood and/or fecal sample; bacterial PGN in a fecal sample; PGLYRP1 in a blood and/or fecal sample; and a TREM1 gene activation signature in diseased tissue and/or blood.

In some embodiments of provided methods of treatment, the one or more activated neutrophil and/or monocyte biomarker(s) and/or one or more biomarker(s) associated with activated neutrophils and/or monocytes is detected in a subject blood sample, diseased tissue sample, fecal matter sample, components or fractions thereof, or combinations thereof.

In some embodiments of provided methods of treatment, the anti-inflammatory therapy (e.g., a TREM1 antibody agent and/or composition as disclosed herein) is specifically targeted to a tissue expressing one or more activated neutrophil and/or monocyte biomarker(s) and/or one or more biomarker(s) associated with (i.e., is a proxy for) activated neutrophils and/or monocytes. In some embodiments, the anti-inflammatory therapy (e.g., a TREM1 antibody agent and/or composition as disclosed herein) is specifically targeted to a tissue expressing elevated levels of the one or more activated neutrophil and/or monocyte biomarker(s), and/or one or more biomarker(s) associated with (i.e. is a proxy for) activated neutrophils and/or monocytes, as compared to levels of the one or more inflammatory biomarker(s), and/or one or more biomarker(s) associated with activated neutrophils and/or monocytes, in the subject when the subject does not have the inflammatory disease, disorder or condition, or when the subject does not have an active form of the disease, disorder or condition. In some embodiments, the anti-inflammatory therapy (e.g., a TREM1 antibody agent and/or composition as disclosed herein) is specifically targeted to a tissue expressing elevated levels of one or more activated neutrophil and/or monocyte biomarker(s), and/or one or more biomarker(s) associated with (i.e. is a proxy for) activated neutrophils and/or monocytes, as compared to levels of the one or more inflammatory biomarker(s), and/or one or more biomarker(s) associated with activated neutrophils and/or monocytes in the general population. For example, in some embodiments, the anti-inflammatory therapy is specifically targeted to small intestine tissue (e.g., tissue of the duodenum, jejunum, or ileum), large intestine (e.g., cecum, ascending colon, transverse colon, or descending colon), and/or rectal tissue expressing, or expressing elevated levels of, the one or more activated neutrophil and/or monocyte biomarker(s), and/or one or more biomarker(s) associated with (i.e., is a proxy for) activated neutrophils and/or monocytes.

In some embodiments of provided methods of treatment, the subject having an inflammatory disease, disorder, or condition is treated with a TREM1 antibody agent and/or composition as disclosed herein in combination with one or more one or more alternative anti-inflammatory therapies that do not directly target TREM1 (e.g., one or more TNFα inhibitor(s)). For example, in some embodiments, a subject is treated with a TREM1 antibody agent and/or composition as disclosed herein) in combination with an anti-TNFα antibody agent (e.g., an anti-TNFα monoclonal antibody, an anti-TNFα humanized antibody, and/or an anti-TNFα chimeric antibody). In some embodiments, a subject is treated with TREM1 inhibitor in combination with adalimumab, certolizumab, golimumab, infliximab, and/or etanercept.

In some embodiments, a TREM1 antibody agent or composition of the present disclosure is administered to a subject who has received, or is receiving, an alternative therapy. For example, in some embodiments, the alternative therapy is an alternative IBD therapy such as an anti-TNFα therapy (e.g., infliximab, adalimumab, golimumab, and certolizumab pegol); a JAK inhibitor therapy (including selective and non-selective inhibitors) (e.g., upadacitinib, tofacitinib and filgotinib); an anti-integrin therapy (e.g., vedolizumab and natalizumab); an anti-IL23 therapy (e.g., guselkumab, mirikizumab, and brazikumab); an anti-IL-12/23 therapy (e.g., ustekinumab); an anti-IL-23A therapy (e.g., risankizumab); an S1PR agonist or modulator therapy (e.g., ozanimod and etrasimod); a 5-aminosalicylate therapy (e.g., mesalamine, olsalazine, balsalazide, and sulfasalazine); an immunomodulatory therapy (e.g., azathioprine, 6-mercaptopurine, and Methotrexate); a corticosteroid therapy (e.g., prednisone, methylprednisolone, hydrocortisone, and budesonide), an anti-TL1A therapy (e.g., PRA023 and RVT-3101); a kinase inhibitor (e.g., ritlecitinib), a TYK2 inhibitor therapy (e.g., deucravacitinib), an anti-IL-36 therapy (e.g., spesolimab); an anti-IL-13 therapy (e.g., dupilumab), a miR-124 upregulator therapy (e.g., obefazimod), a TLR9 agonist therapy (e.g., cobitolimod); or a combination thereof.

### Methods of Production

TREM1 antibody agents or portions thereof, of the present disclosure may be produced using a recombinant expression method as commonly known by persons of skill in the art. For example, a nucleic acid encoding the TREM1 antibody agent, or portion thereof, is isolated and inserted into a replicable vector for further cloning and amplification, or for expression. DNA encoding the antibody or portion thereof is isolated and sequenced using conventional procedures (e.g., by using oligonucleotide probes that are capable of binding specifically to genes encoding the heavy and light chains of the antibody). Various expression vectors may be used and are known to persons of skill in the art. The choice of vector may depend in part on the host cell to be used for expression. Generally, preferred host cells are of either prokaryotic or eukaryotic (e.g., mammalian) origin. In some embodiments, TREM1 antibody agents, or portions thereof, are produced in a bacterial cell, a yeast cell, an insect cell, or a mammalian cell. In some embodiments, TREM1 antibody agents, or portions thereof, are produced in Chinese hamster ovary (CHO) cells. In some embodiments, TREM1 antibody agents, or portions thereof are produced in cell-free systems as are well known by persons of skill in the art.

In some embodiments, TREM1 antibody agents, or portions thereof described herein are characterized by an ability to be produced at a concentration of at least 3000 mg/mL. In some embodiments, TREM1 antibody agents, or portions thereof described herein are characterized by an ability to be produced at a concentration of about 3.5, about 4, about 4.5, about 5, about 5.5, about 6, about 6.5, about 7, about 7.5, about 8, about 8.5, about 9, about 9.5, or about 10 g/L. In some embodiments, TREM1 antibody agents, or portions thereof described herein are characterized by an ability to be produced at a concentration of about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5 g/L. In some embodiments, TREM1 antibody agents, or portions thereof described herein are characterized by an ability to be produced at a concentration of about 5.12, about 5.13, about 5.18, or about 5.7 g/L.

Compositions and antibody agents disclosed herein may be used for treatment of a disease, disorder, or condition associated with cells expressing TREM1 and/or any downstream inflammatory mediators triggered by TREM1 activation. In some embodiments, a disease, disorder, or condition to be treated may be selected from a disease, disorder, or condition disclosed herein. In some embodiments, a disease, disorder, or condition to be treated may be an inflammatory disorder, e.g., a condition associated with unwanted inflammation or with an increase in inflammation. In some embodiments, the disease to be treated is inflammatory bowel disease (IBD).

### Detection of Activated Immune Cells

Circulating neutrophils are generally quiescent, until activation is triggered, e.g., through exposure to one or more external signals (e.g., pathogens, chemical signals, proteins, etc.). Without wishing to be bound by any particular theory, we note that neutrophils may undergo a multistep activation process, entering a partially activated state through exposure to one or more initial signals and then a fully activated state (referred to interchangeably herein as an “activated state”) upon exposure to one or more additional signals. Activated neutrophils may, among other things, produce signaling molecules (e.g., cytokines, chemokines, degranulating enzymes, etc.) in order to initiate an amplified immune response in a subject.

Inflammatory monocytes sense their environments and can be activated through exposure to one or more external signals (e.g., pathogens, etc.). Inflammatory monocytes selectively traffic to sites of inflammation, produce inflammatory cytokines and chemokines, and contribute to local and systemic inflammation. Without wishing to be bound by any particular theory, we note that inflammatory monocytes may undergo a multistep activation process, entering a partially activated state through exposure to one or more initial signals and then a fully activated state (referred to interchangeably herein as an “activated state”) upon exposure to one or more additional signals. Activated inflammatory monocytes may, among other things, produce signaling molecules (e.g., cytokines, chemokines, etc.) in order to promote an amplified immune response in a subject.

The present disclosure teaches that presence and/or level of monocytes (e.g., inflammatory monocytes) and/or neutrophils (e.g., activated neutrophils) may be useful in the context of anti-inflammatory therapy. In particular, the present disclosure provides an insight that subjects determined to have elevated levels of monocytes and/or activated neutrophils (e.g., relative to an appropriate reference such as, for example, an established population norm and/or a historical personal reference), may be particularly likely to respond to therapy targeting TREM1 (and/or, relatively less likely to respond to alternative therapies that do not directly target TREM1, e.g., anti-TNFα therapies).

In some embodiments, monocyte and/or neutrophil activation may be triggered by a pathogen. In some embodiments, monocyte and/or neutrophil activation is triggered by a bacterial pathogen, a viral pathogen, or a fungal pathogen.

In some embodiments, monocyte and/or neutrophil activation may be triggered by exposure to one or more bacterial markers (e.g., peptidoglycans). In some embodiments, monocyte and/or neutrophil activation may be triggered by exposure to one or more pathogen-associated molecular patterns (PAMPs). In some embodiments, monocyte and/or neutrophil activation may be triggered by lipopolysaccharide. In some embodiments, monocyte and/or neutrophil activation may be triggered by lipoteichoic acids. In some embodiments, monocyte and/or neutrophil activation may be triggered by double-stranded viral RNA. In some embodiments, monocyte and/or neutrophil activation may be triggered by bacterial DNA.

In some embodiments, monocyte and/or neutrophil activation may be triggered by a damage-associated molecular pattern released by a necrotic cell. In some embodiments, monocyte and/or neutrophil activation may be triggered by a high-mobility group protein B1. In some embodiments, monocyte and/or neutrophil activation may be triggered by a mitochondrial formyl peptide. In some embodiments, monocyte and/or neutrophil activation may be triggered by mitochondrial DNA.

In some embodiments, monocyte and/or neutrophil activation may be triggered by an inflammatory cytokine (e.g., TNFα). In some embodiments, monocyte and/or neutrophil activation may be triggered by a chemokine. In some embodiments, monocyte and/or neutrophil activation may be triggered by a growth factor. In some embodiments, monocyte and/or neutrophil activation may be triggered by adhesion to an endothelial cell. In some embodiments, monocyte and/or neutrophil activation may be triggered by an immune complex. In some embodiments, monocyte and/or neutrophil activation may be triggered by hypoxic conditions.

Monocytes (e.g., inflammatory monocytes) and/or activated neutrophils may be characterized by the presence of one or more markers (e.g., biomarkers and/or proxy markers). In some embodiments, a biomarker and/or proxy marker of activated neutrophils and/or monocytes is a cell-based biomarker. For example, in some embodiments, the cell-based biomarker is a blood and/or tissue neutrophil count, monocyte count, or neutrophil and monocyte count. In some embodiments, the biomarker and/or proxy marker is TREM1 RNA in monocytes and/or neutrophils. In some embodiments, the biomarker or proxy marker is TREM1 on the cell surface of monocytes and/or neutrophils.

In some embodiments, a biomarker and/or proxy marker of activated neutrophils is a tissue biomarker. For example, in some embodiments, the tissue biomarker is mucosal ulceration. In some embodiments, the biomarker and/or proxy marker is the presence of neutrophils in the lamina propria or neutrophils in an epithelial layer of the subject. In some embodiments, a biomarker and/or proxy marker is the presence of lymphocytes, plasmocytes and/or eosinophils in the lamina propria of the subject.

In some embodiments, a biomarker and/or proxy marker of activated neutrophils is a product of neutrophils. For example, in some embodiments, the product of neutrophils is calprotectin, PGLYRP1, or soluble TREM1.

In some embodiments, a biomarker and/or proxy marker of monocytes (e.g., inflammatory monocytes) is a product of monocytes (e.g., inflammatory monocytes). For example, in some embodiments, the product of monocytes (e.g., inflammatory monocytes) is soluble TREM1.

In some embodiments, a biomarker and/or proxy marker of activated neutrophils and/or monocytes is a TREM1 ligand. For example, in some embodiments, the TREM1 ligand is bacterial peptidoglycan (PGN) and/or PGLYRP1.

In some embodiments, a biomarker and/or proxy marker of activated neutrophils and/or monocytes is a TREM1 gene activation signature.

In some embodiments, a biomarker and/or proxy marker of activated neutrophils and/or monocytes includes any one or combination of: a neutrophil and/or monocyte count detected in diseased tissue; TREM1 on the cell surface of monocytes and/or neutrophils in diseased tissue; TREM1 RNA in monocytes and/or neutrophils in diseased tissue; a tissue biomarker in diseased tissue; soluble TREM1 in a blood and/or fecal sample; calprotectin in a blood and/or fecal sample; bacterial PGN in a fecal sample; PGLYRP1 in a blood and/or fecal sample; and a TREM1 gene activation signature in diseased tissue and/or blood.

In some embodiments, a biomarker and/or proxy marker of activated neutrophils and/or monocytes is detected in a subject blood sample, diseased tissue sample, fecal matter sample, components or fractions thereof, or combinations thereof.

In some embodiments, the elevated levels of activated neutrophils and/or monocytes is determined by measuring the presence of elevated levels of one or more activated neutrophil and/or monocyte biomarker(s), and/or one or more biomarker(s) associated with (i.e., is a proxy for) activated neutrophils and/or monocytes, as compared to levels of the one or more inflammatory biomarker(s), and/or one or more biomarker(s) associated with activated neutrophils and/or monocytes, in a subject when the subject does not have the inflammatory disease, disorder or condition, or when the subject does not have an active form of the disease, disorder or condition. In some embodiments, the elevated levels of activated neutrophils and/or monocytes is determined by measuring the presence of elevated levels of one or more activated neutrophil and/or monocyte biomarker(s), and/or one or more biomarker(s) associated with (i.e., is a proxy for) activated neutrophils and/or monocytes in a subject, as compared to levels of the one or more inflammatory biomarker(s), and/or one or more biomarker(s) associated with activated neutrophils and/or monocytes in the general population. In some embodiments, the elevated levels of activated neutrophils and/or monocytes is determined by measuring the presence of elevated levels of one or more activated neutrophil and/or monocyte biomarker(s), and/or one or more biomarker(s) associated with (i.e., is a proxy for) activated neutrophils and/or monocytes in a diseased tissue sample collected from a subject, as compared to levels of the one or more inflammatory biomarker(s), and/or one or more biomarker(s) associated with activated neutrophils and/or monocytes in a healthy tissue sample collected from the same subject.

In some embodiments, activated neutrophils and/or monocytes, activated neutrophil and/or monocyte biomarkers, and/or biomarkers associated with (i.e., that are a proxy for) activated neutrophils and/or monocytes may be detected through one or more methods available in the art. For example, in some embodiments, activated neutrophils and/or monocytes may be detected through one or more microscopy methods (e.g., fluorescence microscopy). In some embodiments, activated neutrophils and/or monocytes, activated neutrophil and/or monocyte biomarkers, and/or biomarkers associated with (i.e. is a proxy for) activated neutrophils and/or monocytes may be detected through one or more histopathology methods. In some embodiments, activated neutrophils and/or monocytes, activated neutrophil and/or monocyte biomarkers, and/or biomarkers associated with (i.e. is a proxy for) activated neutrophils and/or monocytes may be detected through one or more polynucleotide (e.g. DNA or RNA) detection methods (e.g., quantitative PCR, reverse-transcription PCR, in situ hybridization, in vitro hybridization, nucleic acid sequencing, etc.). In some embodiments, activated neutrophils and/or monocytes, activated neutrophil and/or monocyte biomarkers, and/or biomarkers associated with (i.e. is a proxy for) activated neutrophils and/or monocytes may be detected through one or more spatial profiling methods. In some embodiments, activated neutrophils and/or monocytes, activated neutrophil and/or monocyte biomarkers, and/or biomarkers associated with (i.e. is a proxy for) activated neutrophils and/or monocytes may be detected through one or more methods for protein identification and/or quantification (e.g., antibody-based detection, ELISA, competitive binding studies, mass spectrometry and/or high-pressure liquid chromatography of a tissue sample collected from a subject, etc.). In some embodiments, activated neutrophils and/or monocytes, activated neutrophil and/or monocyte biomarkers, and/or biomarkers associated with (i.e. is a proxy for) activated neutrophils and/or monocytes may be detected through one or more cell sorting or characterization methods (e.g., FACS, flow cytometry, etc.).

### EXAMPLES

The following examples are provided so as to describe to the skilled artisan how to make and use methods and compositions described herein, and are not intended to limit the scope of the present disclosure.

### Example 1: Elevated TREM1 Positive Inflammatory Monocytes and Neutrophils in Inflamed Tissues

The present Example includes the identification of elevated inflammatory monocytes and neutrophils in patients with inflammatory bowel disease (IBD). Inflammatory monocytes were enriched in inflamed tissues, enriched in post-treatment samples from non-responders to anti-TNFα therapy, as compared to responders to anti-TNFα therapy, and showed elevated TREM1 expression.

Single cell transcriptomics data were generated from approximately 200 tissues samples (comprising over 1 million cells) from inflammatory bowel disease (IBD) patients that were naïve to treatment with biologic therapy. Samples were collected from multiple anatomical locations both prior to treatment and after at least 3-months of treatment with adalimumab, an anti-TNFα therapy. The samples were annotated with anti-TNFα therapy responder status and inflammation status.

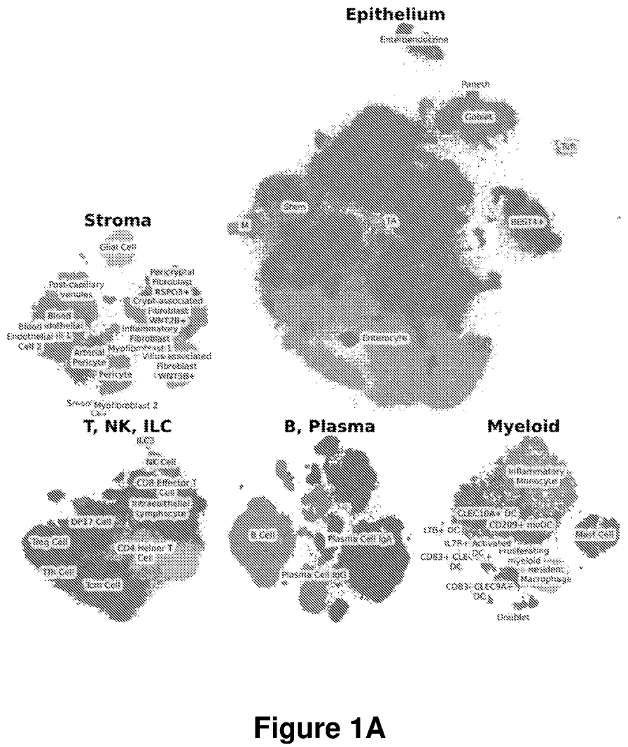

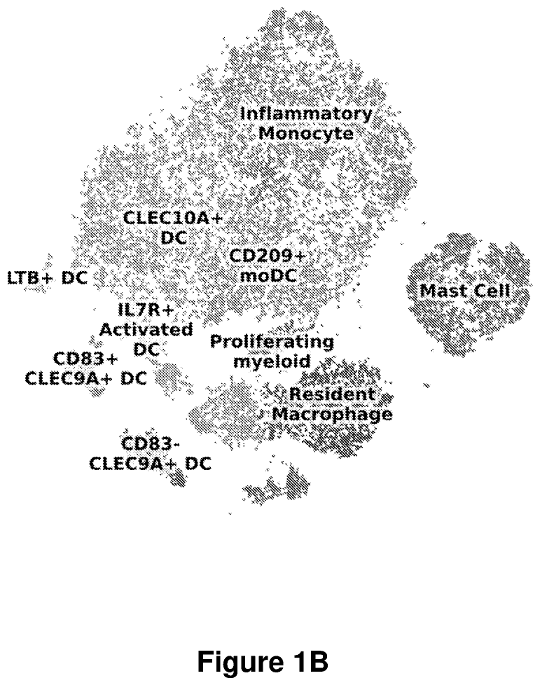

The single cell transcriptomics data was clustered into cell populations that were annotated with cell identities (FIGS. 1A and 1B). The cell populations were then analyzed for differences across clinical dimensions (e.g., anti-TNFα therapy responder status and/or inflammation status).

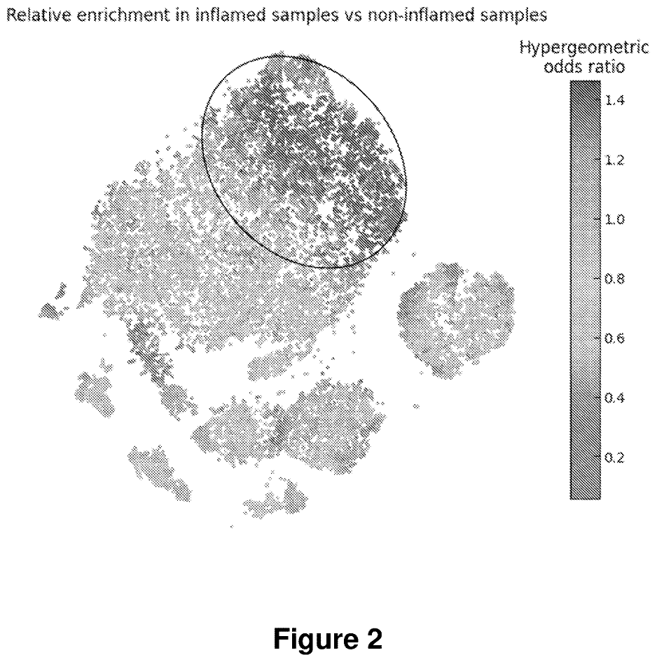

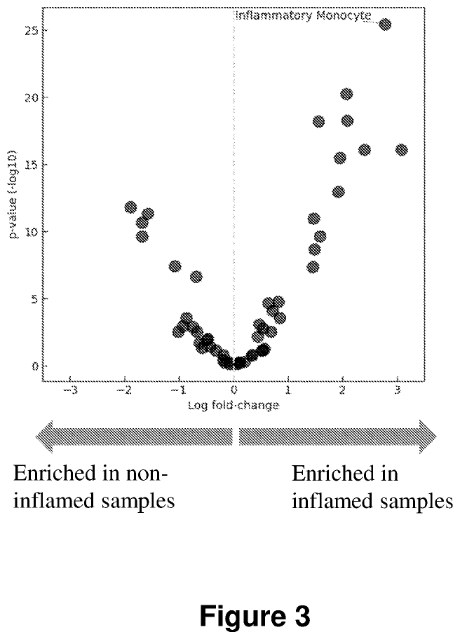

Single cell profiling revealed that certain cell populations (e.g., inflammatory monocytes) were enriched in inflamed samples compared to non-inflamed samples (FIGS. 2 and 3).

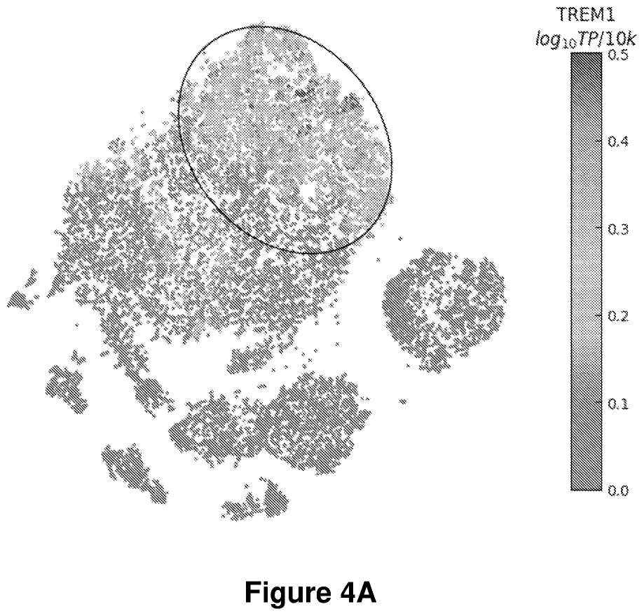

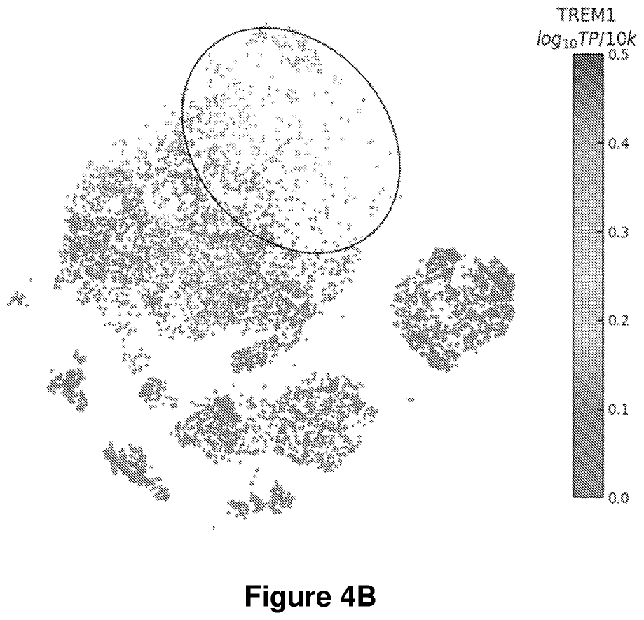

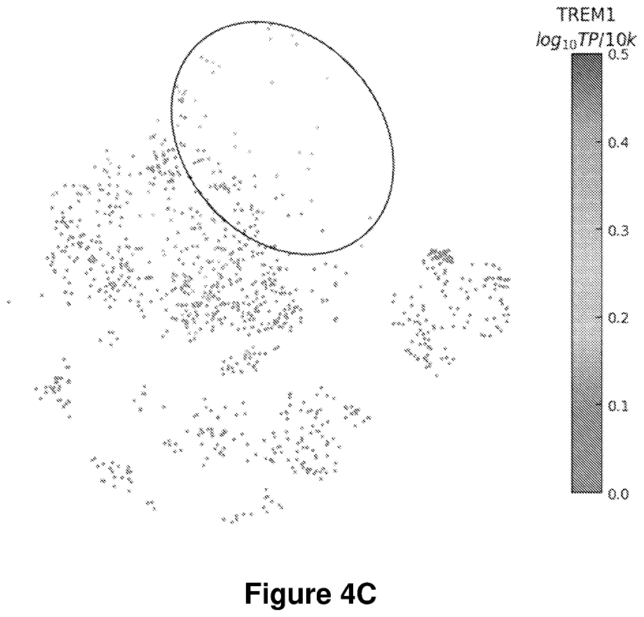

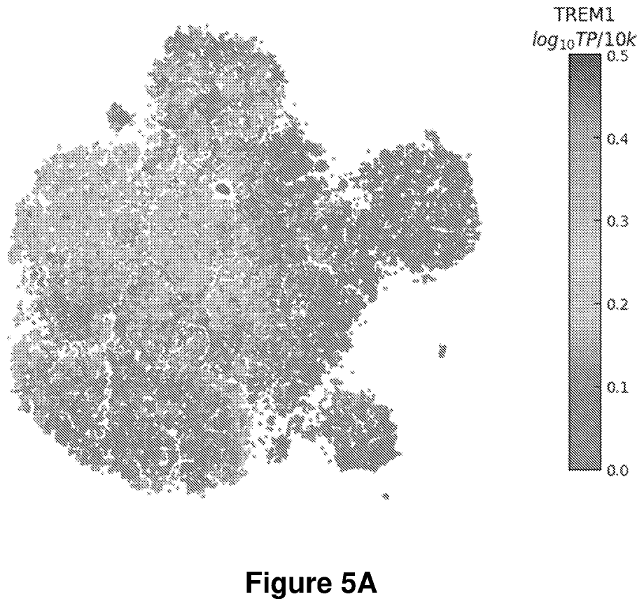

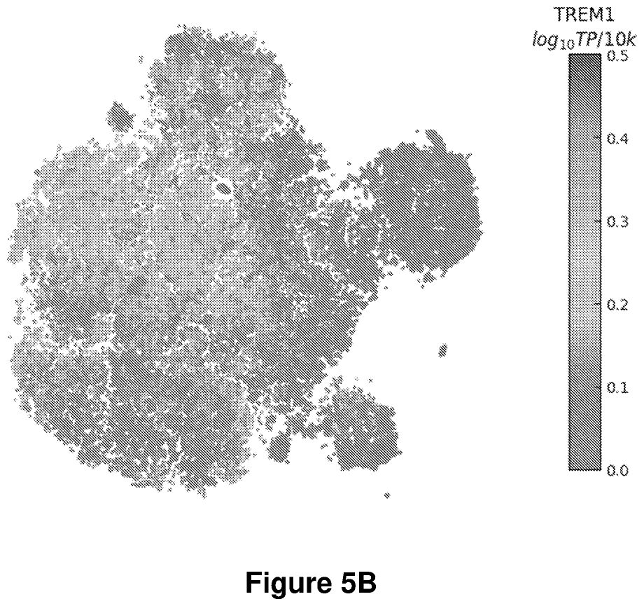

Analysis showed that TREM1 is highly expressed in inflammatory monocytes present in inflamed tissues from IBD patients (FIG. 4A). In contrast, TREM1 positive inflammatory monocytes were not enriched in non-inflamed tissues from IBD patients (FIG. 4B) or in healthy tissues from non-IBD patients (FIG. 4C). TREM1 positive monocytes were also not enriched in peripheral blood mononuclear cells (PBMCs) from IBD patients compared to healthy individuals (FIGS. 5A and 5B).

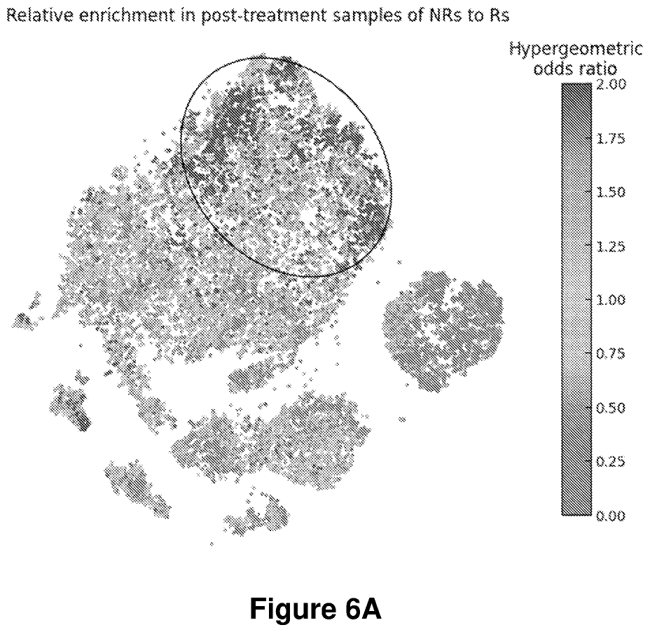

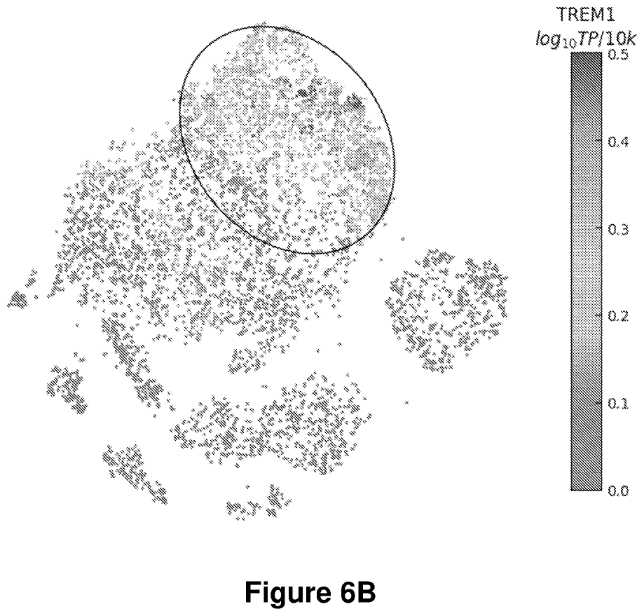

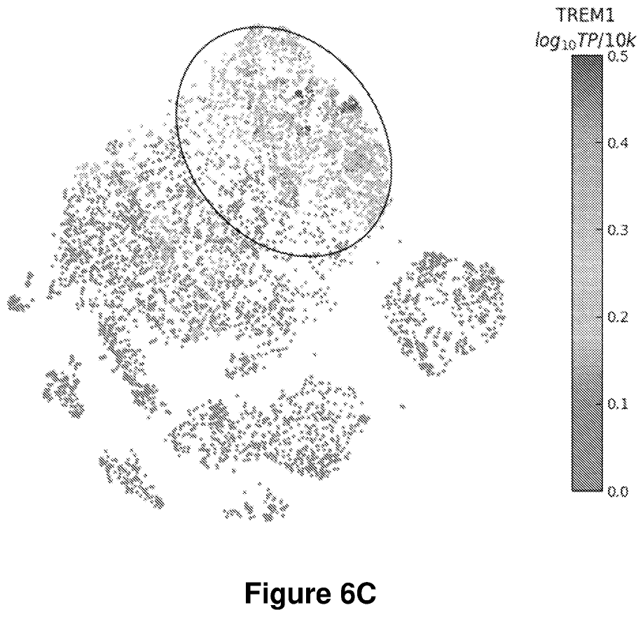

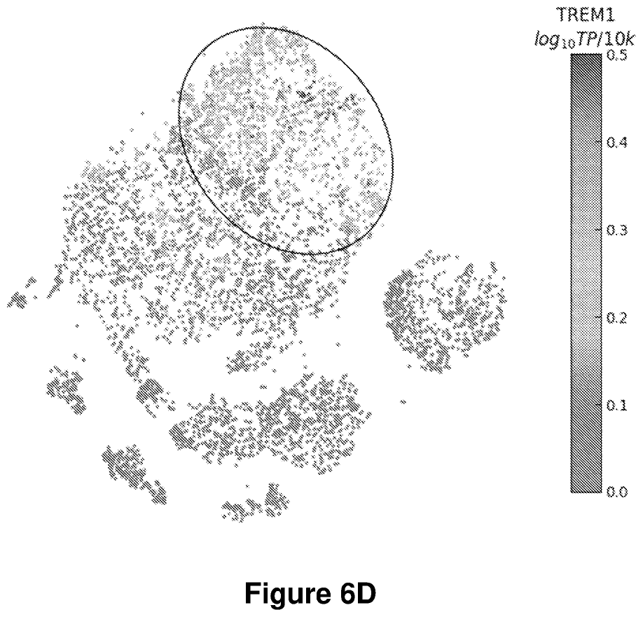

Inflammatory monocytes were enriched in post-treatment samples from the tissues of IBD patients that are non-responders to anti-TNFα therapy as compared to post-treatment samples from IBD patients that are responders to anti-TNFα therapy (FIG. 6A). Inflammatory monocytes in post-treatment samples from tissues of IBD patients that are non-responders to anti-TNFα therapy displayed high levels of TREM1 (FIG. 6B). High TREM1 inflammatory monocytes were enriched in tissues of both Crohn's disease (CD) and ulcerative colitis (UC) IBD patients that are non-responders to anti-TNFα therapy (FIGS. 6C and 6D).

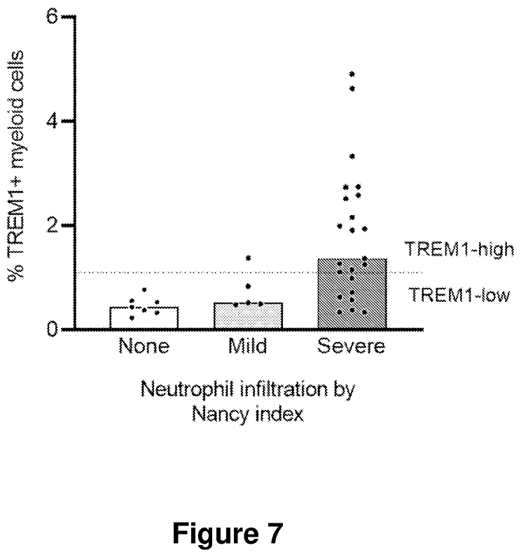

Enrichment of TREM1+ inflammatory monocytes in inflamed tissues correlated with neutrophil infiltration of the inflamed tissues, as assessed by histology, in patients with ulcerative colitis (FIG. 7). Neutrophils also express high levels of TREM1 (see Example 2).

These results demonstrate that inflammatory monocytes and neutrophils are enriched in inflamed IBD tissues, and that these cell populations highly express TREM1 and are enriched in tissues of IBD patients that are non-responders to anti-TNFα therapy relative to responders to anti-TNFα therapy.

### Example 2: Anti-TREM1 Antibody Agent Binds to Cells that Express TREM1

The present Example demonstrates that an exemplary anti-TREM1 antibody agent disclosed herein is able to bind to TREM1 expressed on the cell surface of monocytes and neutrophils. The exemplary anti-TREM1 antibody agent has amino acid sequences as disclosed in Table 2 as corresponding to clone A. Additionally, the present Example indicates that bacterial components (e.g., PGN or LPS) can stimulate monocytes to upregulate cell surface levels of TREM1.

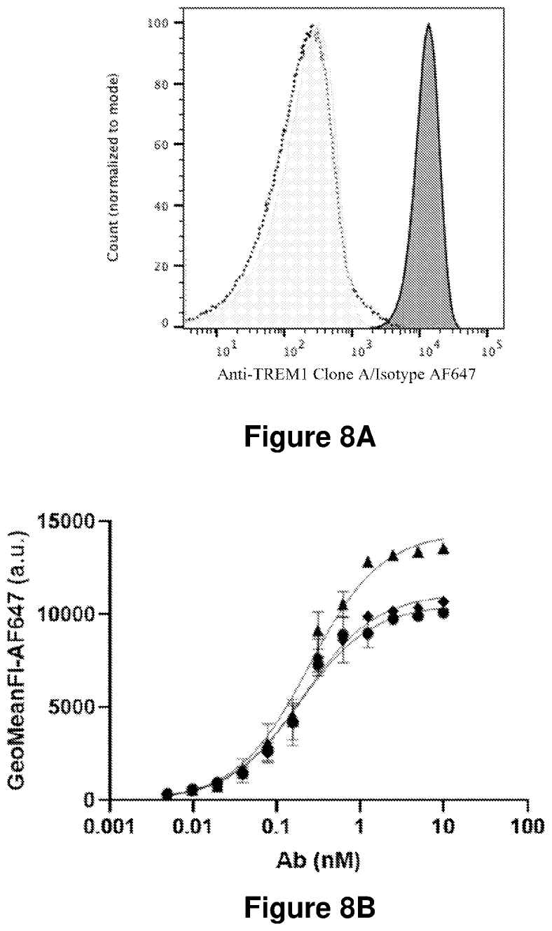

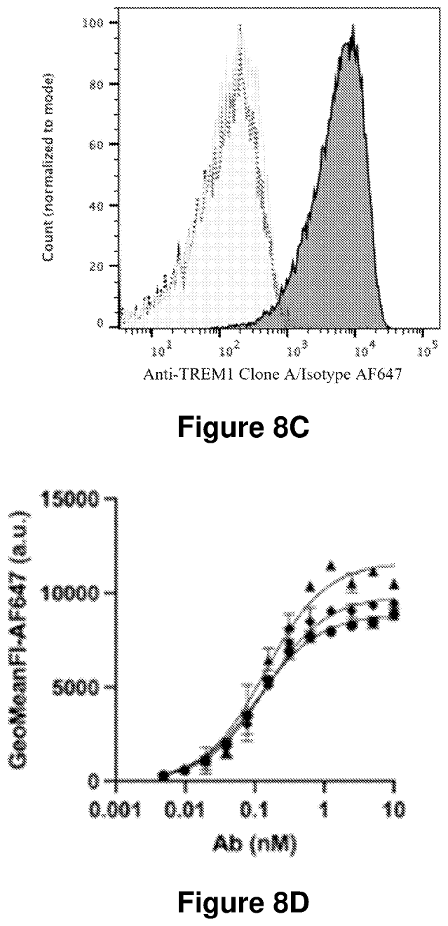

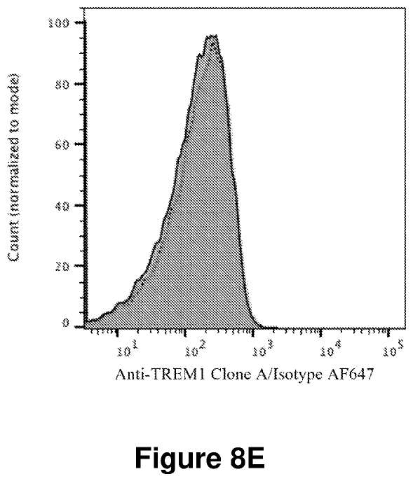

Binding of anti-TREM1 antibody agent clone A to cell surface TREM1 was analyzed by staining of cells present in whole blood samples from a healthy human subject followed by flow cytometry analysis. Briefly, whole blood samples were depleted of red blood cells (RBCs) by sedimentation and then Fc blocked before being stained using various antibodies against various cell surface markers directly conjugated to fluorophores. The cells were also stained with saturating concentration (10 nM) of anti-TREM1 antibody agent clone A directly conjugated to Alexa Fluor 647. Staining with an isotype control antibody conjugated to Alexa Fluor 647 was used as a negative control. The stained samples were then analyzed using flow cytometry. Different cell types were identified using the cell surface markers and cell surface TREM1 staining intensities were assessed for each cell type. Neutrophils (CD45⁺ CD66⁺ CD16⁺) and total CD14⁺ monocytes (CD45⁺ CD14⁺ CD16⁻ or CD45⁺ CD14⁺ CD16⁻), which are both known to express TREM1, showed binding of anti-TREM1 antibody agent clone A (FIGS. 8A through 8D), while CD45+Lin⁺(CD3⁺ CD7⁺ CD20⁺) cells (corresponding to T cells, NK cells, and B cells), which are not thought to express TREM1, did not show binding of anti-TREM1 antibody clone A (FIG. 8E). As the cells were not exposed to bacterial peptidoglycan (PGN) or PGLYRP1 prior to staining, it is expected that TREM1 present on the cell surfaces is in a non-activated state. These results demonstrate that TREM1 is selectively expressed on the cell surface of neutrophils and monocytes and that anti-TREM1 antibody agent clone A binds to TREM1 (e.g., non-activated TREM1) present on the cell surface of neutrophils and monocytes.

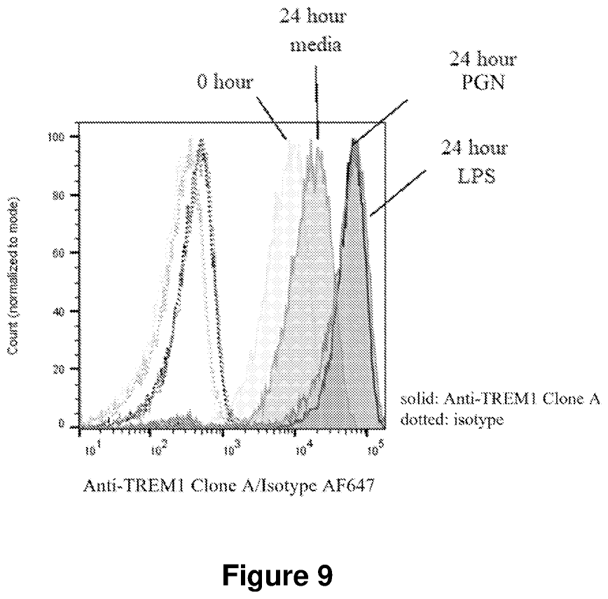

To determine whether anti-TREM1 antibody agent clone A is able to bind to activated TREM, human primary monocytes from healthy donors were plated and stimulated with bacterial peptidoglycan (PGN) (10 μg/mL) or LPS (100 ng/mL) for 24 hours, followed by staining with saturating concentration of anti-TREM1 antibody agent clone A (5 nM) and flow cytometry analysis. PGN stimulation and LPS stimulation resulted in upregulation of cell surface TREM1 (FIG. 9). A modest increase in cell surface TREM1 was observed in monocytes at 24 hours without PGN (24 hours media), consistent with low-level activation of the monocytes due to plating. An isotype control antibody was used as a control for anti-TREM1 staining. These results demonstrate that monocyte activation by PGN or LPS results in upregulation of TREM1 on the cell surface. Without wishing to be bound by any particular theory, these results may be consistent with bacterial components present in diseased tissues promoting upregulation of TREM1 on the cell surface of monocytes. The results also demonstrate that anti-TREM1 antibody agent clone A is able to bind activated TREM1 present on the surface of cells.

### Example 3: TREM1 Amplifies Inflammatory Responses Mediated by TREM1 Positive Monocytes and Neutrophils

The present Example demonstrates that TREM1 amplifies inflammatory responses mediated by TREM1 positive monocytes and neutrophils.

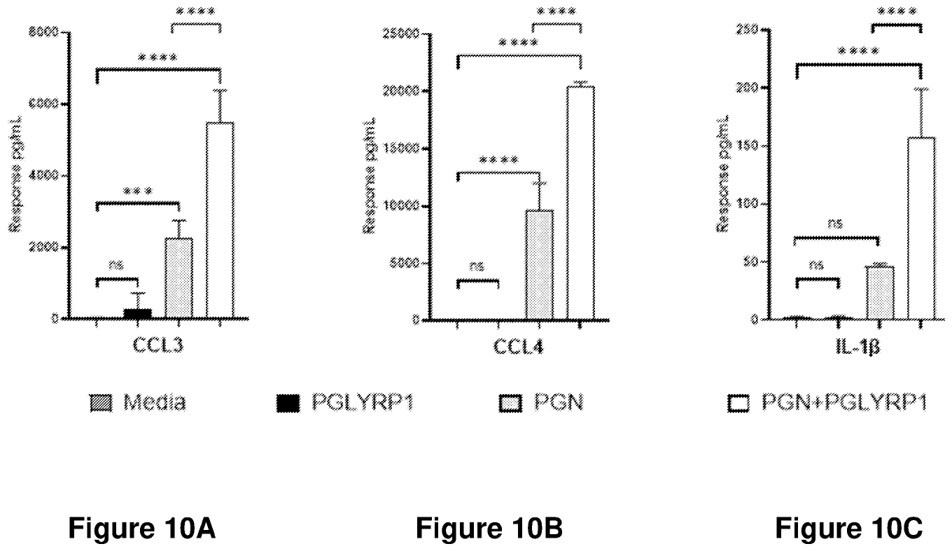

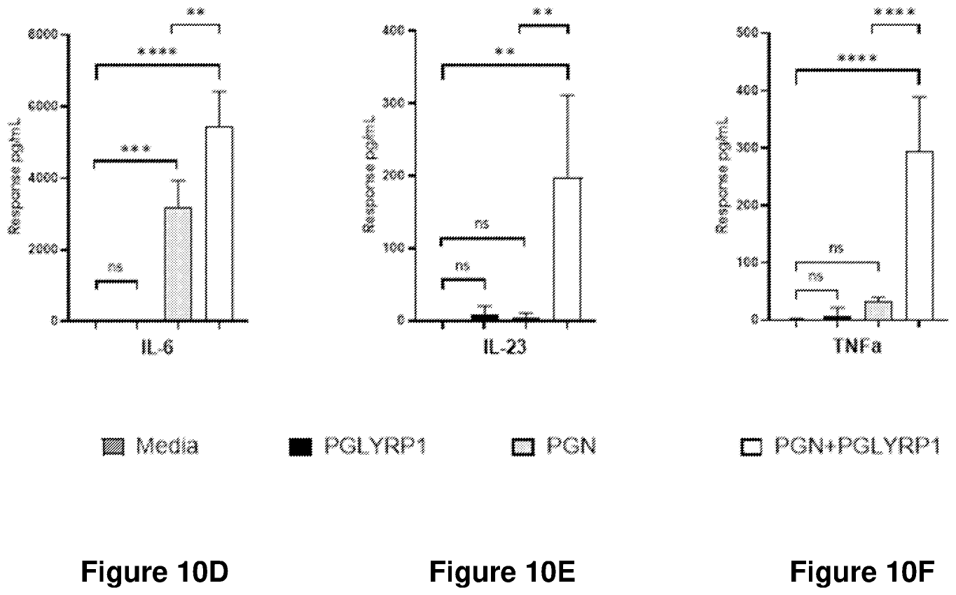

Primary human monocytes from healthy volunteers were isolated and stimulated with PGLYRP1 alone (500 nM), bacterial PGN alone (100 ng/mL), or a combination of PGN (100 ng/mL) and PGLYRP1 (500 nM). Unstimulated monocytes (media) were used as an additional control to show baseline cytokine levels. After 18-24 hours, monocyte mediated inflammatory responses were monitored by assaying the levels of various cytokines and chemokines (e.g., CCL3, CCL4, IL-1β, IL-6, IL-23, and TNFα) secreted into the culture media. The results showed that PGLYRP1 alone does not stimulate monocytes to secrete the assayed inflammatory cytokines and chemokines (FIG. 10). In contrast, PGN alone significantly stimulated monocytes to secrete several of the assayed cytokines and chemokines (e.g., CCL3, CCL4, and IL-6) (FIG. 10). The combination of PGN with PGLYRP1 significantly amplified the secretion of the assayed inflammatory cytokines and chemokines relative to PGN alone (FIG. 10). These results demonstrate bacterial PGN stimulates monocytes to produce an initial anti-bacterial innate immune response that includes the secretion of inflammatory cytokines and chemokines that is amplified by the TREM1 ligand PGLYRP1.

In a similar experiment, inflammatory responses mediated by primary human monocytes were monitored by assaying the levels of various inflammatory mediators using a 64-plex assay panel (data not shown), which demonstrated that TREM1 amplifies the secretion by monocytes of a variety of inflammatory mediators including, but not limited to, e.g., inflammatory cytokines (e.g., IL-1β, IL-6, IL-23, and TNFα), chemokines (e.g., CCL2, CCL3, CCL4, CCL8, CCL20, CCL22, CCL24, CXCL1, CXCL5, CXCL9, and CXCL13), activators of B cell, T cells, and/or other cells (e.g., APRIL, BAFF, CD30, and M-CSF), and factors implicated in epithelial barrier integrity (e.g., TRAIL, TWEAK, MMP-1, IL-20, and TNFR-II).

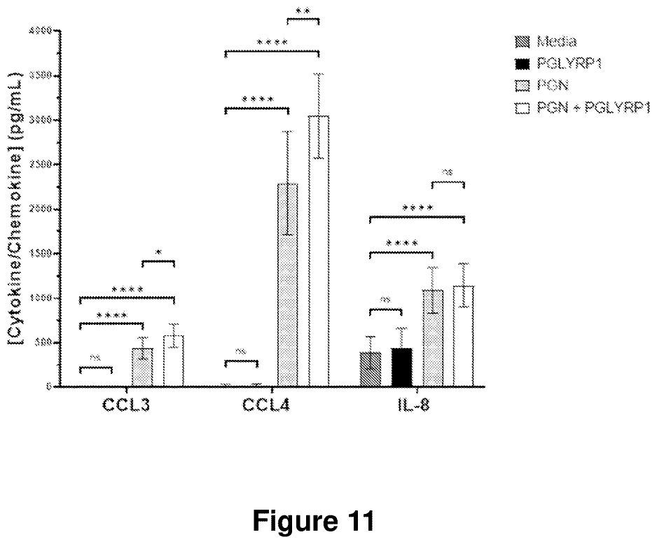

Primary human neutrophils from healthy volunteers were isolated and stimulated with PGLYRP1 alone (50 nM), bacterial PGN alone (300 ng/mL), or a combination of PGN (300 ng/mL) and PGLYRP1 (50 nM). Unstimulated neutrophils (media) were used as an additional control to show baseline cytokine levels. After 18-24 hours, neutrophils mediated inflammatory responses were monitored by assaying the levels of various cytokines and chemokines (e.g., CCL3, CCL4, and IL-8) secreted into the culture media. The results showed that PGLYRP1 alone does not stimulate neutrophils to secrete the assayed inflammatory cytokines and chemokines (FIG. 11). In contrast, PGN alone significantly stimulated neutrophils to secrete the assayed cytokines and chemokines (e.g., CCL3, CCL4, and IL-8) (FIG. 11). Neutrophils release endogenous PGLYRP1 in response to stimulation by PGN. The combination of PGN with PGLYRP1 significantly amplified the secretion of CCL3 and CCL4 relative to PGN alone (FIG. 11). These results demonstrate bacterial PGN stimulates neutrophils to produce an initial anti-bacterial innate immune response that includes the secretion of inflammatory cytokines and chemokines that is amplified by the TREM1 ligand PGLYRP1.

### Example 4: Characterization of an Anti-TREM1 Antibody Agent

The present Example provides characterization of an exemplary anti-TREM1 antibody agent disclosed herein. The exemplary anti-TREM1 antibody agent is a monoclonal antibody that specifically binds to TREM1 and inhibits TREM1 activity. The exemplary anti-TREM1 antibody agent has amino acid sequences as disclosed in Table 2 as corresponding to clone A.

The binding affinity of the TREM1 antibody agent to TREM1 extracellular domain (ECD) from various species was examined using bio-layer interferometry (BLI). Briefly, the TREM1 antibody agent (5 μg/mL) was used to immobilized on an anti-human Fc capture (AHC) sensor. Sensors were monitored to establish a baseline. Sensors were then exposed to recombinant monovalent TREM1-ECD proteins of human, cynomolgus monkey (cyno), dog, rabbit, rat, mouse, or pig at 4 concentrations (0.03 125, 0.0625, 0.25, and 1 μg/mL) for 300 seconds for on-rate measurement, and afterwards were transferred to assay buffer for at least 900 seconds for off-rate measurement.

Table 4 shows results of the BLI experiment. TREM1-ECD proteins with a maximum BLI response less than 0.1 nm had insufficient association response for K_{D} determination and thus are considered to have no significant binding to TREM1 antibody agent clone A. The results demonstrate that the TREM1 antibody agent binds to the ECD of human and cyno TREM1. Binding to the ECD of dog, rabbit, rat, mouse, and pig TREM1 was not detected. The coefficient of determination of the 1:1 model fit to the BLI sensorgrams is represented by the R\hat{ }2 value. Data for human and cynomolgus monkey represents averages across three replicates. Data for pig, dog, rabbit, rat, and mouse represents averages across two replicates.

TABLE 4
Results of BLI assays

| TREM1-ECD | Maximum BLI | | | | |
|---|---|---|---|---|---|
| species | response (nm) | K_{D} (nM) | Kₐ (1/Ms) | K_{dis} (1/s) | R\hat{ }2 |
| Human | 0.385 ± 0.012 | 0.21 ± 0.10 | 4.21 × 10⁵ | 9.20 × 10⁻⁵ | >0.99 |
| Cynomolgus | 0.343 ± 0.008 | 1.90 ± 0.46 | 3.92 × 10⁵ | 7.70 × 10⁻⁴ | >0.99 |
| monkey | | | | | |
| Pig | 0.013 ± 0.012 | n/a | n/a | n/a | n/a |
| Dog | 0.006 ± 0.001 | n/a | n/a | n/a | n/a |
| Rabbit | 0.005 ± 0.001 | n/a | n/a | n/a | n/a |
| Rat | 0.018 ± 0.003 | n/a | n/a | n/a | n/a |
| Mouse | 0.005 ± 0.003 | n/a | n/a | n/a | n/a |

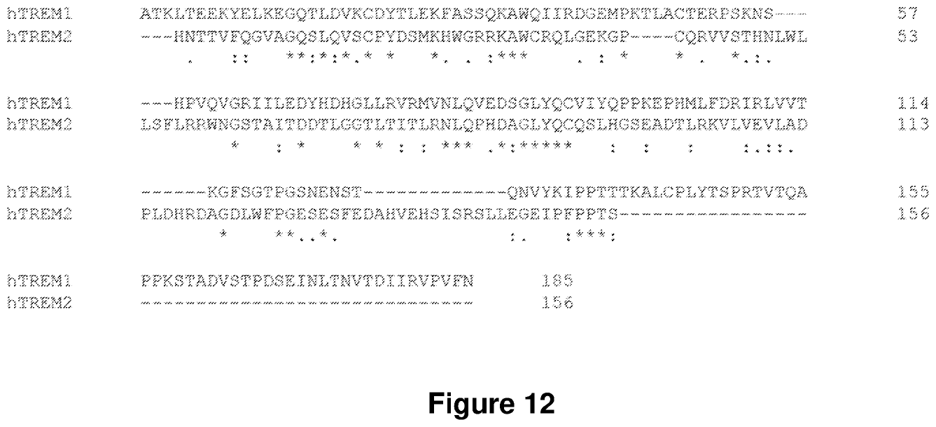

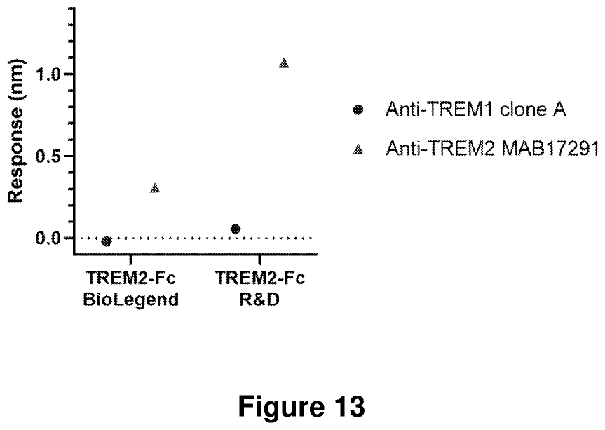

In addition to TREM1, the TREM gene family also includes TREM2. The ECDs of TREM1 and TREM2 have 25% sequence identity to each other (FIG. 12). Without wishing to be bound by any particular theory, TREM1 and TREM2 are thought to play opposing roles in modulating immune responses; see e.g., Sharif and Knapp, Immunobiology, 213(9-10):701-713 (2008). To determine whether the TREM1 antibody agent is also capable of binding to TREM2, bio-layer interferometry (BLI) assay was performed. Briefly, 10 μg/mL TREM2-ECD fused to an Fc domain was immobilized on a sensor. Sensors were quenched and monitored to establish a baseline. Sensors were then exposed to a solution containing 3 μg/mL of the anti-TREM1 antibody agent, or, as a positive control, an anti-TREM2 antibody agent (R&D Systems, MAB17291). The maximum BLI response was measured (FIG. 13). The results demonstrate that the exemplary anti-TREM1 antibody agent does not bind to TREM2-ECD.

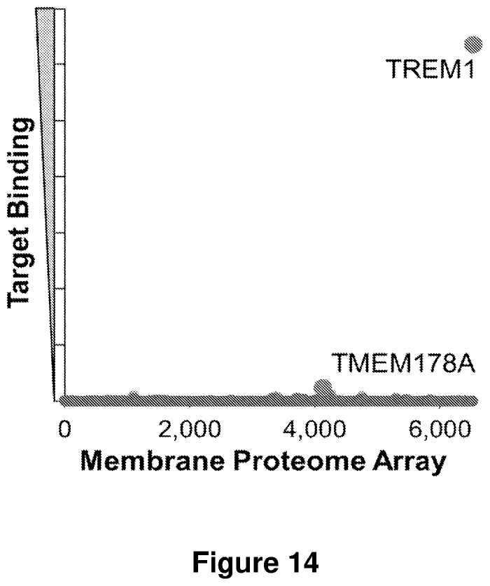

A polyspecificity assay was used to further evaluate the target specificity profile of the anti-TREM1 antibody agent. Transient transfection was used to express an array of each of more than 6,000 human membrane proteins including TREM2 and other TREM family members on the surface of HEK293 cells. The HEK293 cells were stained using 5 μg/mL of the anti-TREM1 antibody agent followed by 3.75 μg/mL of a secondary antibody conjugated to Alexa Fluor 647 (Jackson ImmunoResearch, 109-606-008), and then examined using flow cytometry. The polyspecificity assay showed strong on-target binding of the anti-TREM1 antibody agent to TREM1 (FIG. 14). Only one of the tested human membrane proteins (TMEM178A) showed weak off-target binding that was close to background, while the other tested membrane proteins did not show target binding over background (FIG. 14). TMEM178A is an intracellular protein that localizes to the endoplasmic reticulum; see e.g., Decker et al., Proc. Natl. Acad. Sci., 112(51):15654-9 (2015). The identification of TMEM178A by the polyspecificity assay was not considered biologically relevant, as no anti-TREM1 clone A binding to cells endogenously expressing TMEM178A was detected using flow cytometry.

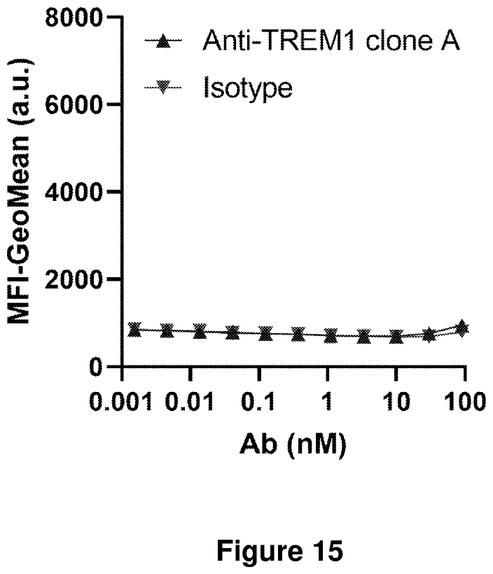

To confirm that the anti-TREM1 antibody agent does not bind to TMEM178A-expressing cells, G401 cells, which are TMEM178A positive and TREM1 negative, were stained with the anti-TREM1 antibody agent conjugated directly to Alexa Fluor 647. Staining with an isotype control antibody conjugated directly to Alexa Fluor 647 was used as a negative control. G401 cells stained with the anti-TREM1 antibody agent or with the isotype control antibody showed comparable fluorescent intensities across a range of antibody concentrations (FIG. 15). These results demonstrate that the anti-TREM1 antibody agent does not appreciably bind to cells expressing TMEM178A.

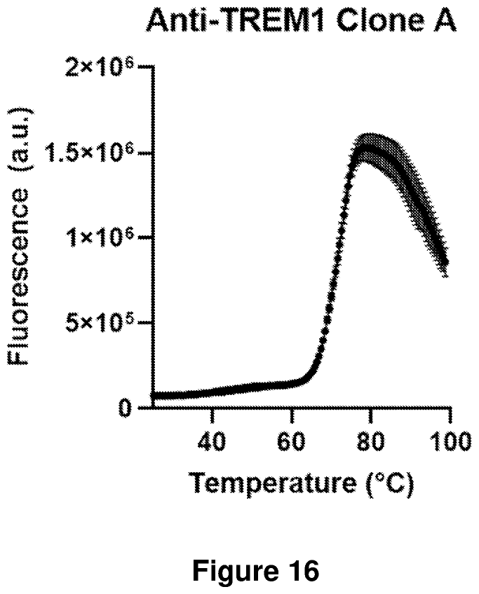

Differential scanning fluorimetry was used to assess the thermostability of the anti-TREM1 antibody agent. Briefly, the antibody agent was incubated with a protein thermal shift dye (Applied Biosystems 4461146) and fluorescence was measured at a range of temperatures using a real-time PCR instrument (Applied Biosystems QuantStudio 6 Flex). FIG. 16 shows the measured fluorescence intensity over a range of temperatures. The first derivative of the fluorescence intensity as a function of temperature can be used to determine the melting temperature (Tₘ) of the antibody agent. The results demonstrate that the anti-TREM1 antibody agent displays a Tₘ of 74° C.

### C1q Binding

To characterize anti-TREM1 antibody agent clone A binding to complement protein C1q, Nunc Maxisorp™ ELISA plates (ThermoFisher Scientific, Loughborough, UK) were coated at 4° C. overnight with serial dilutions of anti-TREM1 antibody agent clone A, Rituximab (positive control), and IgG4 (S241P, L248E) (negative control). Plates were blocked with IX PBS (pH 7.4) with 2% w/v BSA. After blocking, purified human C1q (HyCult Biotech, Wayne, Pennsylvania) at 5 μg/ml was added to the ELISA plates for binding, Plates were washed an anti-C1q-HRP (Abcam, Cambridge, UK) was diluted 1:1000 in blocking buffer and added to the ELISA plates for detection. After washing, TMB microwell peroxidase substrate (Life Technologies) was added for detection. The reaction was stopped with TMB stop solution (VWR, Lutterworth, Leicestershire) after 5 minutes. Absorbance was read at 450 nm using a SpectraMax M3 plate reader (Molecular Devices, Wokingham, UK).

A full dose response curve was obtained for Rituximab and it was determined that Rituximab bound C1q with an EC₅₀ of 1.335 and 1.424 ag/ml for two respective runs. No binding was observed for IgG4 (S241P, L248E) or anti-TREM1 antibody agent clone A.

The present Example demonstrates that an anti-TREM1 antibody agent of the present disclosure inhibits TREM1-mediated inflammatory responses in monocytes and neutrophils.

### Fc Gamma Binding

To characterize anti-TREM1 antibody agent clone A binding to high and low affinity Fc gamma receptors, single cycle analysis using a Biacore T200 instrument was used at a flow rate of 30 μl/min. Human Fc receptors, FcγRI, FcγRII (both 167R and 167H polymorphisms), FcγRIIB, FcγRIIIA (both 176F and 176V polymorphisms) and FcγRIIIB were obtained from Sino Biological (Beijing, China). FcγR were captured on a CM5 sensor chip pre-coupled using a His capture kit (Cytiva, Uppsala, Sweden) using standard amine chemistry.

At the start of each cycle, His-tagged Fcγ receptors diluted in HBS-P+ (Cytiva, Uppsala, Sweden) were loaded to a specific RU level. A five point, three-fold dilution range of test antibody without regeneration between each concentration was used for each receptor tested. Antibodies were passed over the chip in increasing concentrations followed by a single dissociation step. Following dissociation, the chip was regenerated with two injections of glycine (pH 1.5).

Table 5 summarizes the data for anti-TREM1 antibody agent clone A (2 replicates) and Trastuzumab (control) binding to human Fcγ receptors. Single-cycle kinetic analysis shows that there was significant binding of Trastuzumab to both high and low affinity human Fcγ receptors. The binding profiles observed for the 2 replicates of anti-TREM1 antibody agent clone A were similar to one another. Compared with Trastuzumab, anti-TREM1 antibody agent clone A showed reduced binding to all of the low affinity Fcγ receptors with the exception of hFcγRIIA where similar binding was observed. No apparent binding to hFcγRI was observed for anti-TREM1 antibody agent clone A.

TABLE 5
Anti-TREM1 Antibody Agent Clone A FcγR binding

| | hFcγRIIIA_{176F} | hFcγRIIIA_{176V} | hFcγRIIIB | hFcγRIIA_{167R} | hFcγRIIA_{167H} | hFcγRIIB | hFcγRI |
|---|---|---|---|---|---|---|---|
| Trastuzumab | 8.39E−07 | 3.00E−07 | 2.88E−06 | 9.44E−06 | 3.05E−06 | 1.45E−05 | 1.88E−09 |
| K_{D} | | | | | | | |
| Anti- | — | 6.66E−06 | — | 2.84E−05 | 6.21E−06 | — | — |
| TREM1 | | (Ratio | | (Ratio | (Ratio | | |
| Antibody | | 0.05) | | 0.33) | 0.49) | | |
| Clone A | | | | | | | |
| (1) K_{D} | | | | | | | |
| Anti- | — | 6.49E−06 | — | 2.83E−05 | 6.35E−06 | — | — |
| TREM1 | | (Ratio | | (Ratio | (Ratio | | |
| Antibody | | 0.05) | | 0.33) | 0.48) | | |
| Clone A | | | | | | | |
| (2) K_{D} | | | | | | | |

### Antibody-Dependent Cellular Cytotoxicity (ADCC)

The ability of anti-TREM1 antibody agent clone A to elicit antibody-dependent cellular cytotoxicity (ADCC) was examined on a target cell line NCI-H16666, which is a commercially available lung adenocarcinoma cell line and CD20-expressing Raji cells as a positive control, which is a B cell lymphoma line. Enriched human NK cells were used as effector cells from three healthy donors. NK cells were cultured overnight with IL-2 (100 units/ml) prior to usage in ADCC assay. Cells were treated with either anti-TREM1 antibody agent clone A or isotype control antibody at final maximum concentrations of 2000 nM followed by a seven-point log dilution series, in addition to a no-treatment control, in triplicate. Cells were incubated for 15 minutes at 37° C., 5% CO₂, prior to the addition of NK cells. Stimulated NK cells were added to the pre-labeled target cells at an effector to target cell ratio (E:T) of 10:1. Co-cultures were incubated for 4 hours at 37° C., 5% CO₂. Following in vitro culture, plates were washed and centrifuged at 1200 RPM for 3 minutes at room temperature. Supernatants were removed and cells were resuspended in 20 μl of 1× cell staining buffer (Biolegend) with propidium iodide (Invitrogen) at a final concentration of 5 μg/ml. Cells were examined by flow cytometry using an Intellicyt iQue Screener Plus flow cytometer (Sartorius, Albuquerque, NM).

Anti-TREM1 antibody agent clone A did not induce NCI-H1666 ADCC with primary NK cells. No EC₅₀ value was determined due to lack of response.

### Antibody-Dependent Cellular Phagocytosis (ADCP)

The ability of anti-TREM1 antibody agent clone A to elicit antibody-dependent cellular phagocytosis (ADCP) was examined using NCI-H16666 cells and Raji cells as a positive control. Human monocyte-derived in vitro differentiated macrophages from three healthy individual donors were used as effector cells. Human CD14+ monocytes were cultured in human serum and human M-CSF. On day 5, macrophages were harvested using 15 mM lidocaine with 2 mM EDTA solution and counted and assessed for viability prior to and after labeling. Macrophages were labeled with Cell Trace Violet (CTV; ThermoFisher). Target cells were harvested using Accutase (Innovative Cell Technologies, Inc.) prior to labeling with pHrodo Green AM (pHG). 10,000 pHG-labeled target cells were plated per well in a 96-well ultra-low adherence U-bottom tissue culture plate (Corning). Cells were treated with anti-TREM1 antibody agent clone A or isotype control at final maximum concentrations of 2,000 nM followed by a seven-point log dilution series, in addition to a no-treatment control, in triplicate. Cells were incubated for 15 minutes at 37° C., 5% CO₂ prior to the addition of macrophages. CTV-labeled macrophage effector cells were added to pre-labeled target cells at an effector to target ratio (E:T) of 1:2. Co-cultures were incubated for 4 hours at 37° C., 5% CO₂. Following in vitro cell culture, plates were washed and centrifuged at 1200 RPM for 3 minutes at room temperature. Supernatants were removed, and cells were resuspended in 12 μl of 1× cell staining buffer (BioLegened). Cells were examined by flow cytometry using an Intellicyt iQue Screener Plus flow cytometer (Sartorius, Albuquerque, NM).

Anti-TREM1 antibody agent clone A did not induce NCI-H1666 ADCP with monocyte-derived macrophages. No EC₅₀ value was determined due to lack of response.

### Complement-Dependent Cytotoxicity (CDC)

The ability of anti-TREM1 antibody agent clone A to elicit complement-dependent cytotoxicity (CDC) was examined using NCI-H16666 cells and Raji cells as a positive control. Target cells were harvested using Accutase (Innovative Cell Technologies, Inc.) and 50,000 cells were plated per well in a 96-well ultra-low adherence U-bottom tissue culture plate (Corning). Cells were treated with anti-TREM1 antibody agent clone A or isotype control at final maximum concentrations of 2,000 nM followed by a seven-point log dilution series, in addition to a no-treatment control, in triplicate. Cells were incubated for 15 minutes at 37° C., 5% CO₂ prior to treatment with qualified normal human serum (pooled) as a source of complement at a final concentration of 25% v/v, for 2 hours at 37° C., 5% CO₂. Following in vitro cell culture, plates were washed and centrifuged at 1200 RPM for 3 minutes at room temperature. Supernatants were removed, and cells were resuspended in 12 μl of 1× cell staining buffer (BioLegened). Cells were examined by flow cytometry using an Intellicyt iQue Screener Plus flow cytometer (Sartorius, Albuquerque, NM). For analysis, the percentage of propidium iodide-positive cells were determined.

Anti-TREM1 antibody agent clone A did not induce NCI-H1666 CDC with pooled serum. No EC₅₀ value was determined due to lack of response.

Soluble Trem1 (sTREM1)

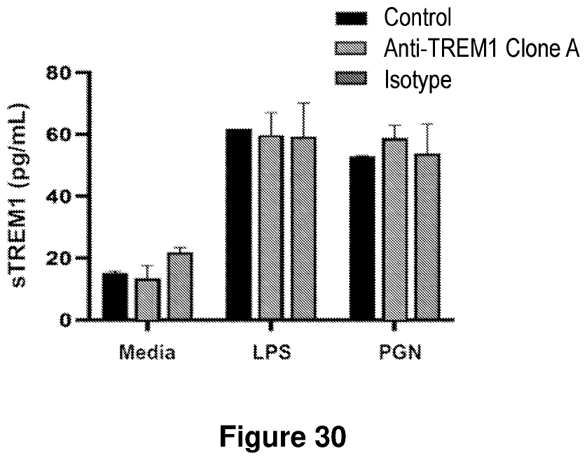

To characterize the potential modulation of soluble TREM1 (sTREM1) levels by anti-TREM1 antibody agent clone A, human monocytes isolated from healthy volunteers were treated with peptidoglycan (PGN; 1 μg/ml) or lipopolysaccharide (LPS; 1 μg/ml) to stimulate increased TREM1 expression on the cell surface or treated with media alone (FIG. 30). Cells were then treated with anti-TREM1 antibody agent clone A or isotype control. As measured by ELISA and shown in FIG. 30, anti-TREM1 antibody agent clone A did not modulate sTREM1 levels produced by monocytes.

### Inflammatory Cytokine Induction

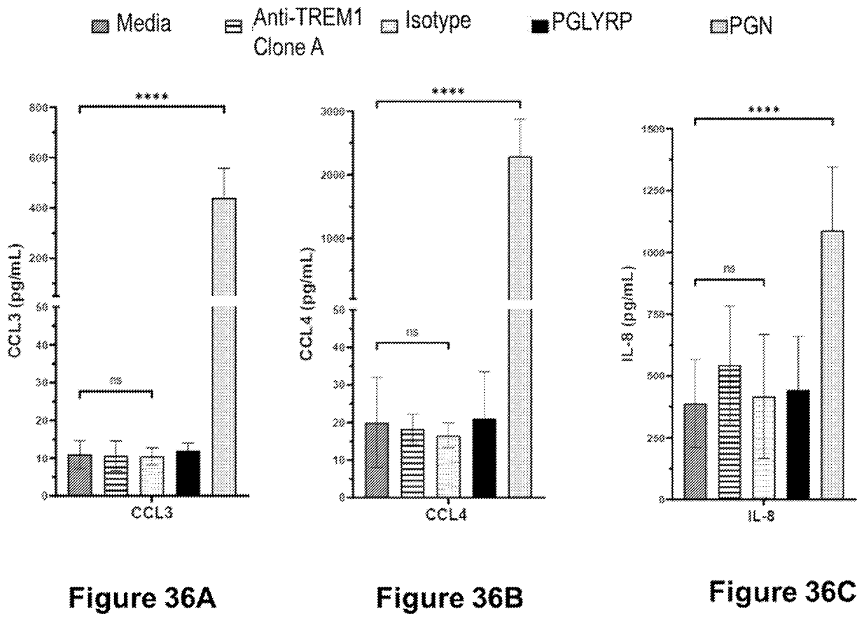

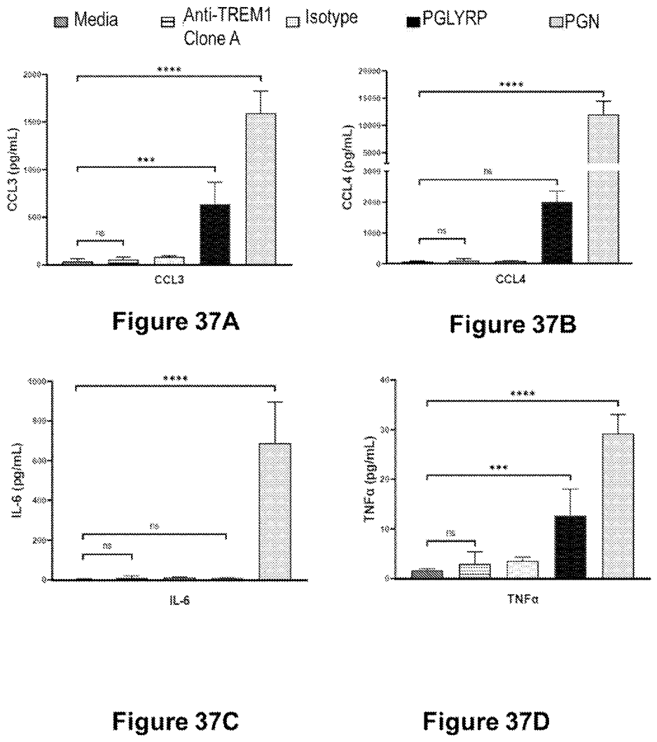

To characterize the potential modulation of inflammatory cytokine and/or chemokine production, primary cells derived from healthy volunteer donors were treated with media alone, 1.25 nM anti-TREM1 antibody agent clone A, 1.25 mM isotype control, 500 nM PGLRYP1, or 100 ng/ml peptidoglycan (PGN). As shown in FIGS. 36A-36C, while primary neutrophils treated for 24 hours with PGLYRP1 or PGN produced elevated levels of inflammatory cytokines and chemokines (e.g., CCL3, CCL4, and IL-8), cells treated with anti-TREM1 antibody agent clone A did not have increased inflammatory cytokine and/or chemokine production and levels were comparable to media-alone. Similarly, as shown in FIGS. 37A-37D primary monocytes treated for 18-24 hours with PGLYRP1 or PGN produced elevated levels of inflammatory cytokines and chemokines (e.g., CCL3, CCL4, IL-6, and TNFα). In contrast, primary monocytes treated with anti-TREM1 antibody agent clone A did not have increased inflammatory cytokine and/or chemokine production.

### Example 5: Anti-TREM1 Inhibits Inflammatory Responses Mediated by TREM1 Positive Monocytes and Neutrophils

The present Example demonstrates that an anti-TREM1 antibody agent of the present disclosure inhibits TREM1-mediated inflammatory responses in monocytes and neutrophils.

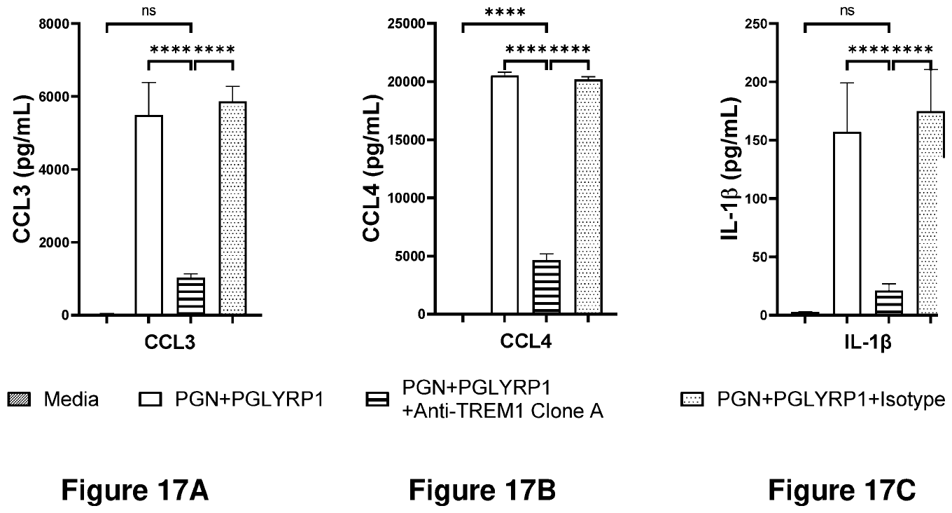

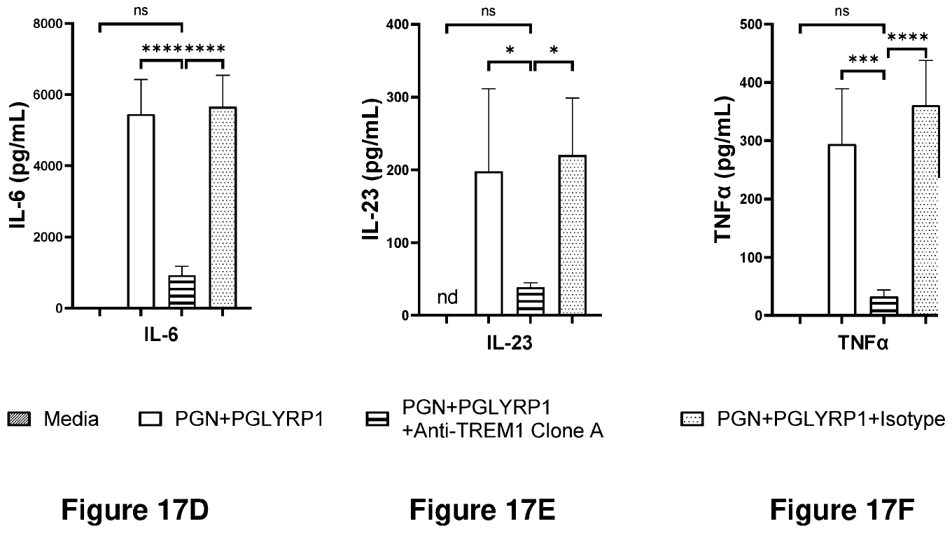

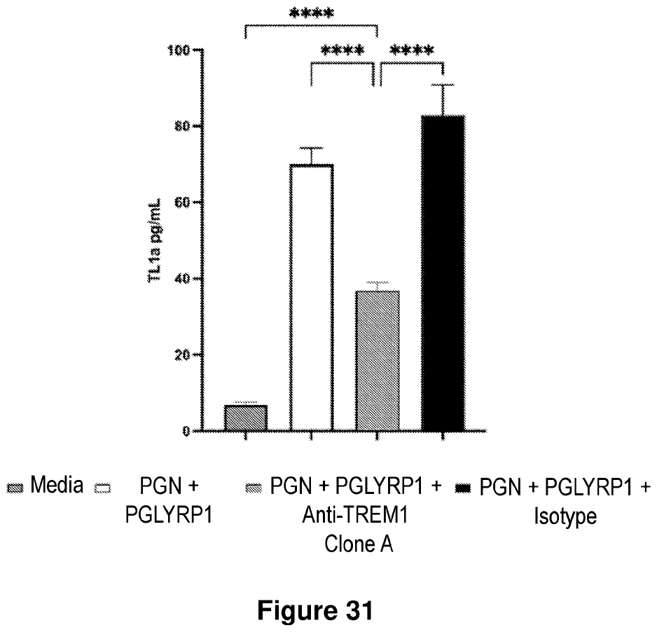

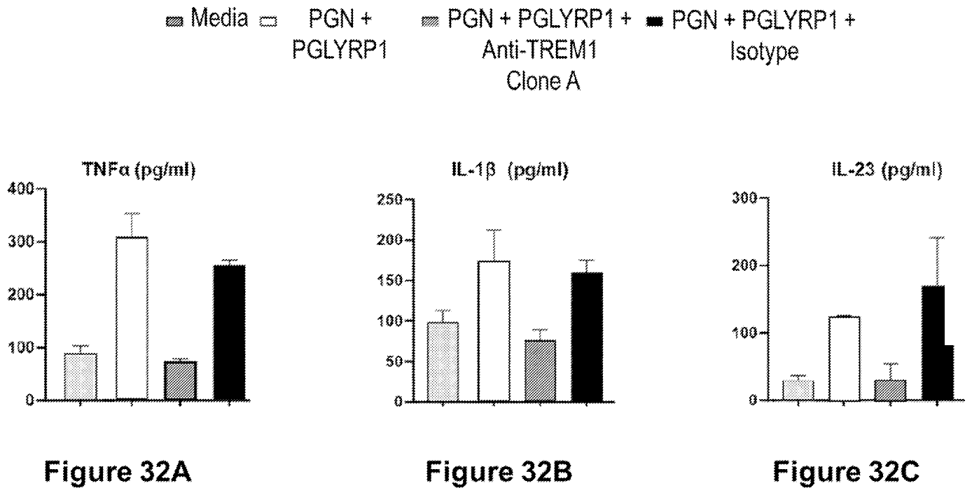

To determine whether TREM1 inhibition can block the amplified inflammatory response mediated by monocytes, primary human monocytes from healthy volunteers were isolated and stimulated with bacterial peptidoglycan (PGN; 100 ng/ml) and PGLYRP1 (500 nM) to elicit an inflammatory response, and then the stimulated monocytes were treated with anti-TREM1 antibody agent clone A (1 nM), with an isotype control (1 nM), or not treated with an antibody agent. Unstimulated monocytes (media) were used as an additional control to show baseline cytokine levels. After 18-24 hours, monocyte mediated inflammatory responses were monitored by assaying the levels of various cytokines and chemokines (e.g., CCL3, CCL4, IL-1β, IL-6, IL-23, TNFα, and TL1A) secreted into the culture media. The results showed that a combination of PGN and PGLYRP1 are able to stimulate TREM1 positive monocytes to secrete each of the assayed inflammatory cytokines, while treatment with the anti-TREM1 antibody agent significantly inhibits secretion of the cytokines (FIGS. 17A-17F and FIG. 31). Treatment with anti-TREM1 antibody agent did not completely inhibit cytokine secretion to baseline levels (FIGS. 17A-17F and FIG. 31), consistent with a role for TREM1 as an amplifier of inflammation. Similarly, as shown in FIGS. 32A-32C, anti TREM1 antibody agent clone A was also able to inhibit TREM-1 pathway activated release of inflammatory cytokines (e.g., TNFα, IL-1β, and IL-23) by primary monocytes derived from tissues collected from ulcerative colitis (UC) patients.

These results demonstrate that TREM1 inhibition blocks an amplified inflammatory response (e.g., as observed in inflammatory diseases), while preserving the initial anti-bacterial innate immune response.

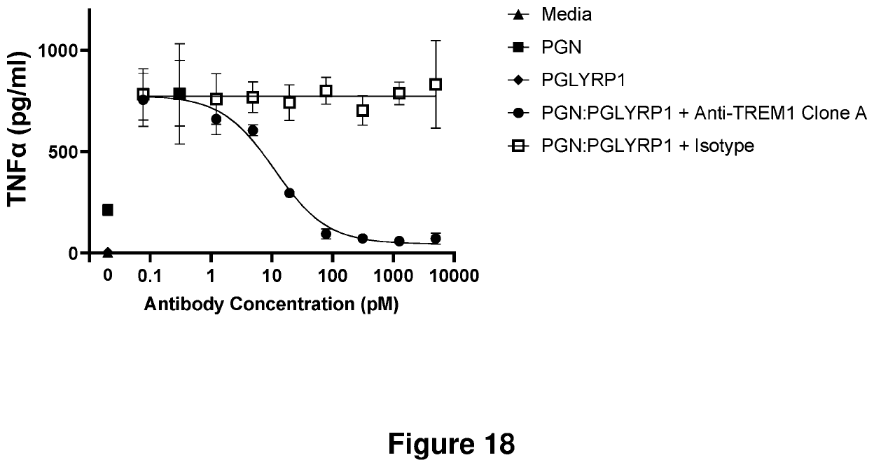

To quantify the functional potency of the anti-TREM1 antibody agent in inhibiting inflammatory responses in monocytes, primary human monocytes from healthy volunteers were isolated and stimulated with bacterial PGN (100 ng/mL), PGLYRP1 (500 nM), or a combination of PGN (100 ng/mL) and PGLYRP1 (500 nM) in the presence of titrated anti-TREM1 antibody agent clone A and isotype control. Unstimulated monocytes (media) were used as an additional control to show baseline cytokine levels. After 18-24 hours, monocyte mediated inflammatory responses were monitored by assaying the levels of TNFα secreted into the culture media. The results showed that the anti-TREM1 antibody agent was able to, in a dose-dependent manner, inhibit TNFα secretion by monocytes in response to stimulation by PGN and PGLYRP1, while isotype control antibody did not inhibit TNFα secretion (FIG. 18). The IC50 was determined by performing a nonlinear curve fit of log(antibody) vs. response (three parameters) and quantitated as 18.1±8.1 pM across 12 healthy volunteers.

Representative IC₅₀ values are shown in Table 6.

TABLE 6
Anti-TREM1 antibody agent clone A inhibition of TREM1-
induced chemokine secretion in primary human monocytes

| | IL-1β | IL-6 | IL-23 | CCL3 | CCL4 | TNFα |
|---|---|---|---|---|---|---|
| Anti-TREM1 | 18.8 ± 8.1 | 24.4 ± 10 | 15.5 ± 8.9 | 21.5 ± 13 | 68.9 ± 60 | 18.1 ± 8.1 |
| antibody | | | | | | |
| agent clone A | | | | | | |
| IC₅₀ (pM) | | | | | | |

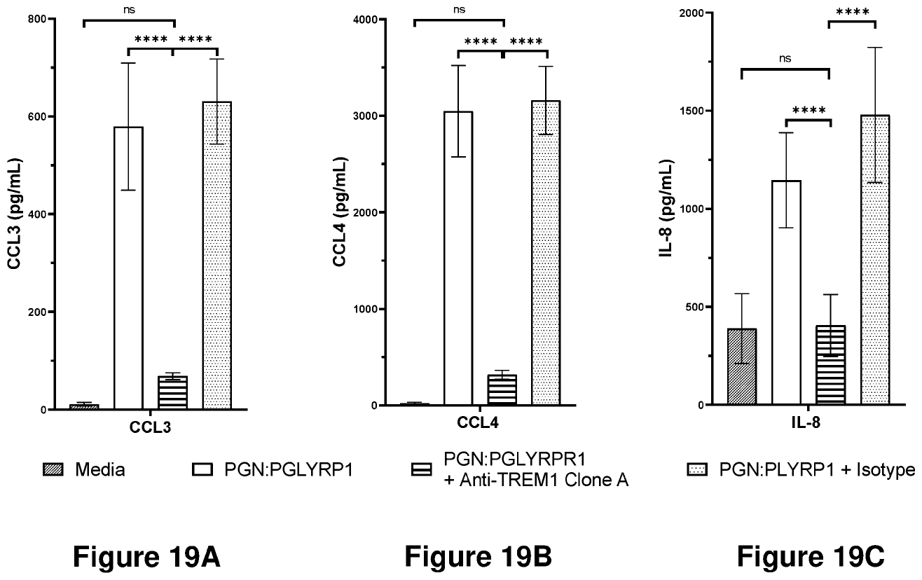

To determine whether TREM1 inhibition can block the amplified inflammatory response mediated by neutrophils, primary human neutrophils from healthy volunteers were isolated and stimulated with a combination of bacterial PGN (300 ng/mL) and PGLYRP1 (50 nM) to elicit an inflammatory response. The stimulated neutrophils were treated with anti-TREM1 antibody agent clone A (2 nM), with an isotype control (2 nM), or not treated with an antibody agent. Unstimulated neutrophils (media) were used as an additional control to show baseline cytokine levels. After 24 hours, neutrophil mediated inflammatory responses were monitored by assaying the levels of various cytokines and chemokines (e.g., CCL3, CCL4, and IL-8) secreted into the culture media. The results showed that a combination of PGN and PGLYRP1 is able to stimulate TREM1 positive neutrophils to secrete each of the assayed inflammatory cytokines, while treatment with the anti-TREM1 antibody agent significantly inhibits secretion of the cytokines without reducing production to the level of media alone (FIGS. 19A-19C). The results showed that PGN and PGLYRP1 induces an amplified inflammatory response and that TREM1 inhibition blocks the amplified inflammatory response (FIGS. 19A-19C).

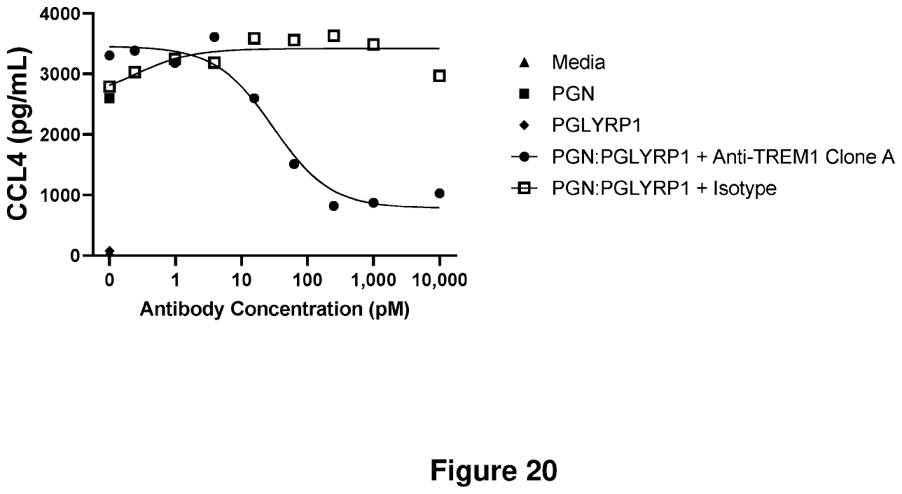

To quantify the functional potency of the anti-TREM1 antibody agent in inhibiting inflammatory responses in neutrophils, primary human neutrophils from healthy volunteers were isolated and stimulated with bacterial PGN (300 ng/mL), PGLYRP1 (50 nM), or a combination of PGN (300 ng/mL) and PGLYRP1 (50 nM) in the presence of titrated anti-TREM1 antibody agent clone A and isotype control. Unstimulated neutrophils (media) were used as an additional control to show baseline chemokine levels. After 18-24 hours, neutrophil mediated inflammatory responses were monitored by assaying the levels of CCL4 secreted into the culture media. The results showed that the anti-TREM1 antibody agent was able to, in a dose-dependent manner, inhibit CCL4 secretion by neutrophils in response to stimulation by PGN and PGLYRP1, while isotype control antibody did not inhibit CCL4 secretion (FIG. 20). The IC50 was determined by performing a nonlinear curve fit of log(antibody) vs. response (three parameters) and quantitated to be 24.9 9.9 pM across 10 healthy volunteers.

Representative IC₅₀ values are shown in Table 7.

TABLE 7
Anti-TREM1 antibody agent clone A inhibition of
TREM1-induced chemokine secretion in neutrophils

| | CCL3 | CCL4 | IL-8 |
|---|---|---|---|
| Anti-TREM1 | 22.3 ± 12.5 | 24.9 ± 9.9 | 22.3 ± 14.5 |
| antibody agent clone | | | |
| A IC₅₀ (pM) | | | |

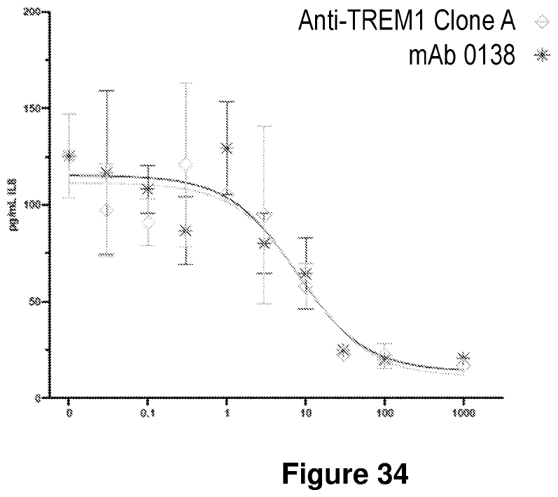

As further shown in FIG. 34, anti-TREM1 antibody agent clone A exhibited similar potency at inhibiting IL-8 production from primary neutrophils as compared to a comparator antibody incorporating the binding domain of mAb 0318 (described in WO2019195126A1, incorporated herein by reference) and the Fc domain of anti-TREM1 antibody agent clone A.

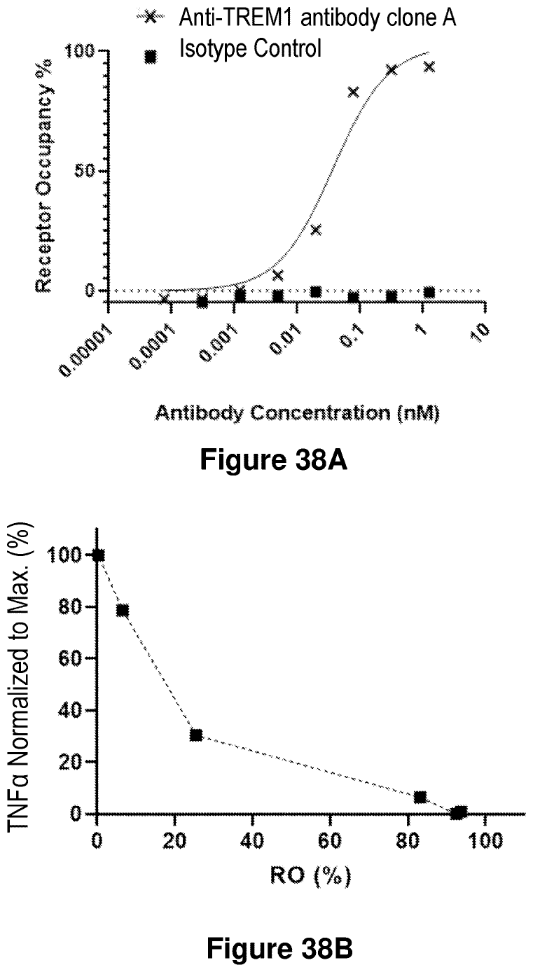

As shown in FIG. 38A, anti-TREM1 antibody agent clone A is capable of saturating TREM1 receptor on the surface of primary monocytes. However, as further shown in FIG. 38B, 100% receptor occupancy of TREM1 expressed on the surface of primary monocytes is not necessary for significant inhibition of inflammatory cytokine production (e.g., TNFα).

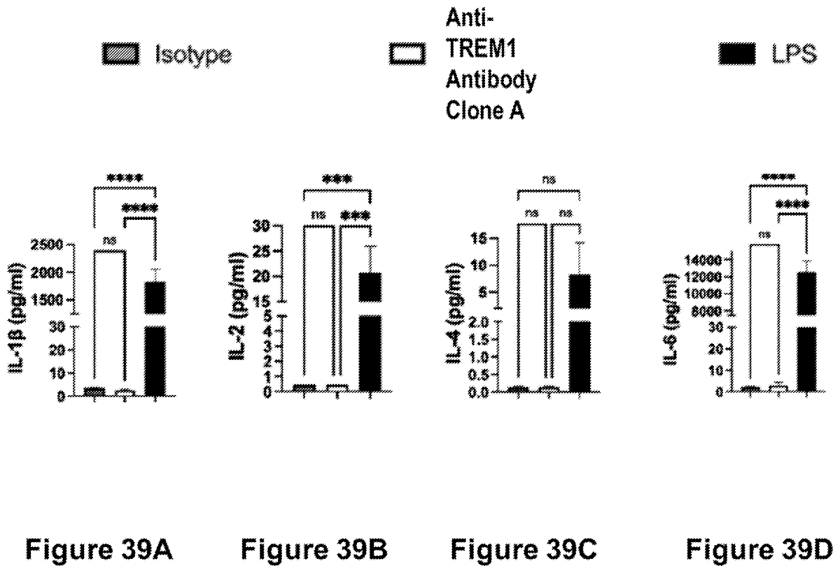

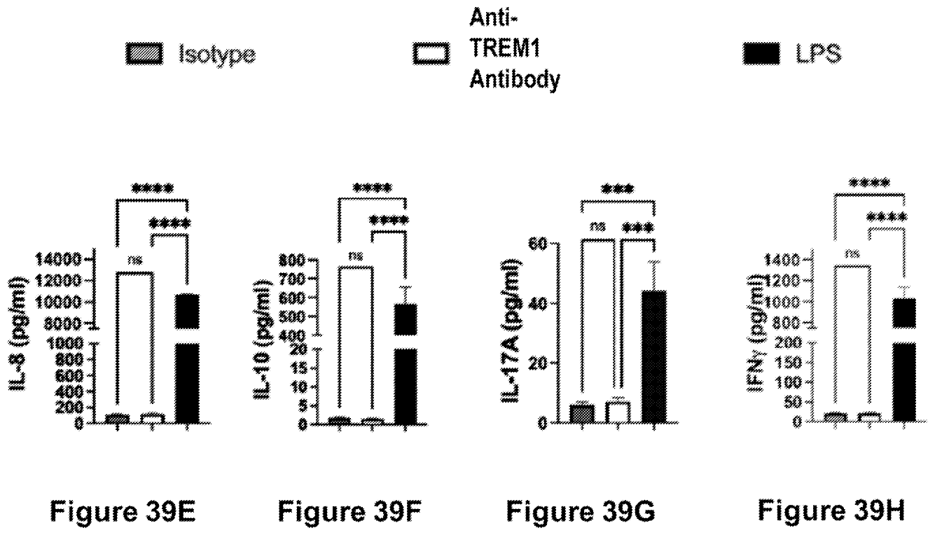

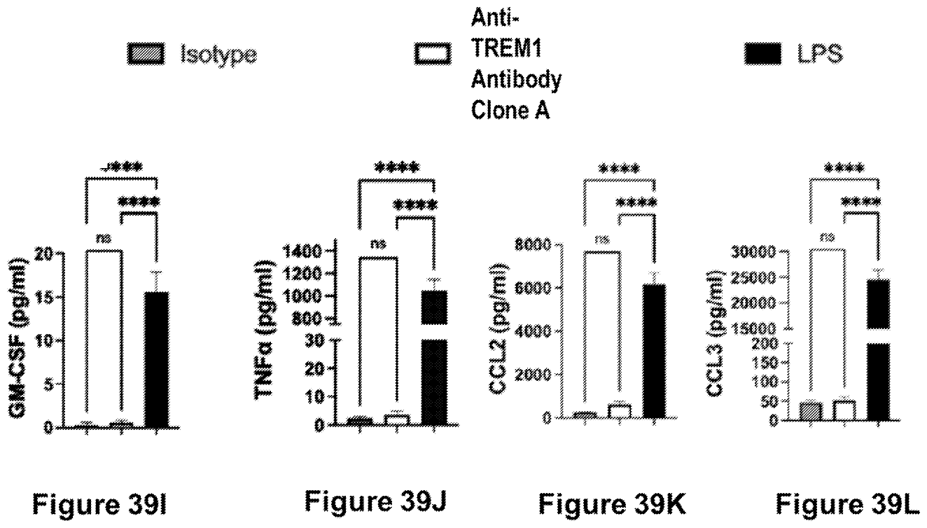

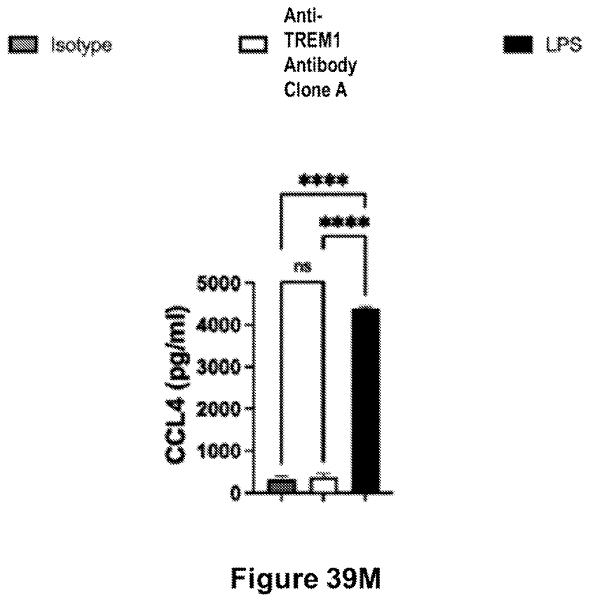

As shown in FIGS. 39A-39M, anti-TREM1 antibody agent clone A is also capable of inhibiting lipopolysaccharide (LPS)-induced inflammatory cytokine and chemokine production in whole blood.

### Example 6: Anti-TREM1 Reduces TREM1-Mediated Epithelial Barrier Damage

The present Example demonstrates that an anti-TREM1 antibody agent of the present disclosure reduces tissue damage (e.g., epithelial barrier damage) caused by TREM1-mediated amplified inflammatory responses.

Epithelial barrier damage was modeled using a trans-epithelial electrical resistance (TEER) assay. TEER assays have been described in the art; see e.g., Srinivasan et al., J. Lab Autom. 20(2):107-126 (2015). In an exemplary TEER assay, an epithelial cell monolayer (e.g., a jejunum human intestinal enteroid monolayer (hIEM)) is grown in a trans-well culture. Formation of an intact epithelial barrier in vitro results in increased trans-epithelial electrical resistance, representing the formation of tight junctions between cells in the monolayer and regulated diffusion between apical and basolateral compartment. Damage to the epithelial barrier is measured as loss of electric resistance across the cell monolayer.

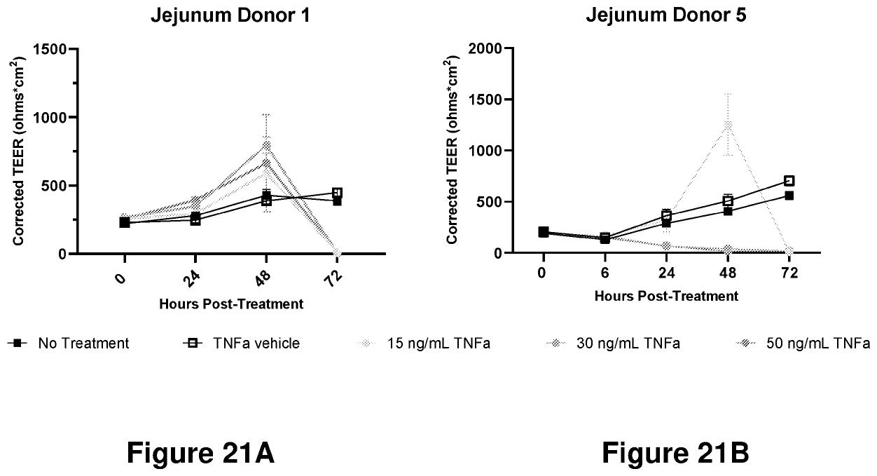

To investigate the effects of TREM1-mediated amplified inflammatory responses on epithelial barrier integrity, an epithelial cell monolayer was exposed to TNFα in a TEER assay. Jejunum cells from each of two donors were cultured in monolayers and the basal compartment was exposed to differentiation media containing various concentrations of TNFα (15 ng/mL, 30 ng/mL, and 50 ng/mL). Jejunum monolayers exposed to differentiation media alone (no treatment) or differentiation media containing vehicle (TNFα vehicle) were used as controls for epithelial barrier formation as measured by elevated TEER measurements. At 24 hours intervals after addition of TNFα, epithelial damage was measured by transepithelial electrical resistance (TEER) and normalized to blank transwell control. The results showed that TNFα results in epithelial barrier damage (FIGS. 21A-21B).

To further investigate the effects of TREM1-mediated amplified inflammatory responses on epithelial barrier integrity, TEER assays were conducted in which epithelial cell monolayers were exposed to conditioned media from monocyte cultures. Primary human monocytes from healthy volunteers were cultured and stimulated with bacterial peptidoglycan (PGN) (100 ng/mL) and PGLYRP1 (500 nM). After 24 hours, culture supernatant was harvested as conditioned media, which is expected to contain soluble mediators of TREM1 amplified immune response (e.g., including, but not limited to, IL-1β, IL-6, IL-23, and TNFα); see also Examples 3 and 5. Conditioned media from unstimulated monocytes was also produced for use as a control. Table 8 shows the measured TNFα levels in conditioned media produced using monocytes from different healthy volunteers. The results showed that depending on the donor, the level of TNFα secreted by monocytes upon stimulation with PGN and PGLYRP1 can by either relatively high (e.g., as observed for Donors 1 and 2) or relatively low (e.g., as observed for Donors 5 and 6).

TABLE 8
TNFα levels in conditioned media produced using monocytes

| | | | TNFα in | |
|---|---|---|---|---|
| | TNFα in | | conditioned | |
| | conditioned | | media from | |
| | media from | | PGN + PGLYRP1 | |
| Monocyte | unstimulated | | stimulated | |
| Donor | monocytes | | monocytes | |
| Donor 1 | 98.4 ± 0 | pg/mL | 5,376.3 ± 202.9 | pg/mL |
| Donor 2 | 63.2 ± 0 | pg/mL | 2,476.9 ± 60.5 | pg/mL |
| Donor 5 | 16.9 | pg/mL | 321.6 | pg/mL |
| Donor 6 | 6.83 | pg/mL | 395.4 | pg/mL |

* Table 8 shows mean and standard deviation for two replicates for Donors 1 and 2, while a single sample was measured for each of Donors 5 and 6.

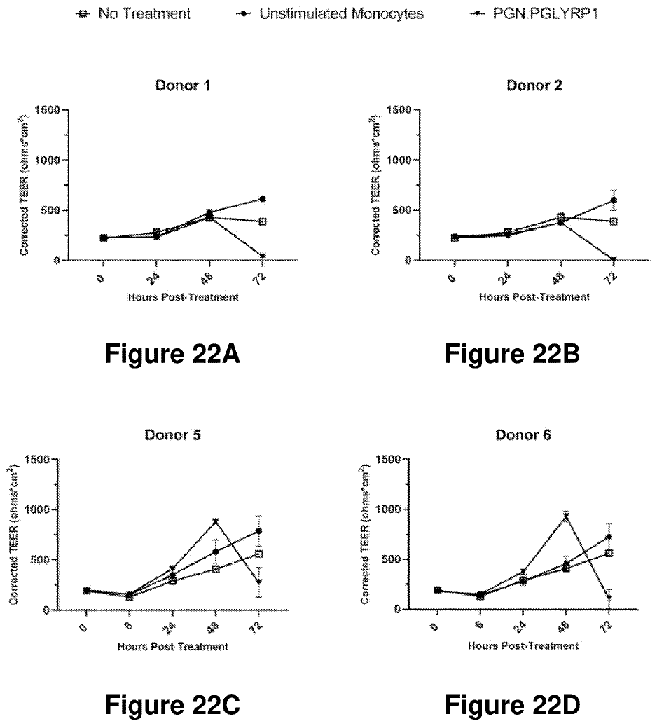

The monocyte conditioned media were then used in TEER assays to investigate their effects on epithelial barrier integrity. Jejunum cells were cultured in a monolayer and the basal compartment was exposed to conditioned media from unstimulated monocytes and monocytes stimulated with PGN and PGLYRP1. No treatment was used as an additional control. At various times (6, 24, 48, and 72 hours) after treatment, epithelial damage was measured by transepithelial electrical resistance (TEER) and normalized to blank transwell control. The results showed that conditioned media from monocytes stimulated with PGN and PGLYPR1 results in epithelial barrier damage (FIGS. 22A-22D). Conditioned media from stimulated monocytes having both high or low levels of TNFα resulted in epithelial barrier damage (FIGS. 22A-22D), indicating that other soluble mediators of TREM1-mediated amplified inflammatory response that are present in the conditioned media also contribute to epithelial barrier damage.

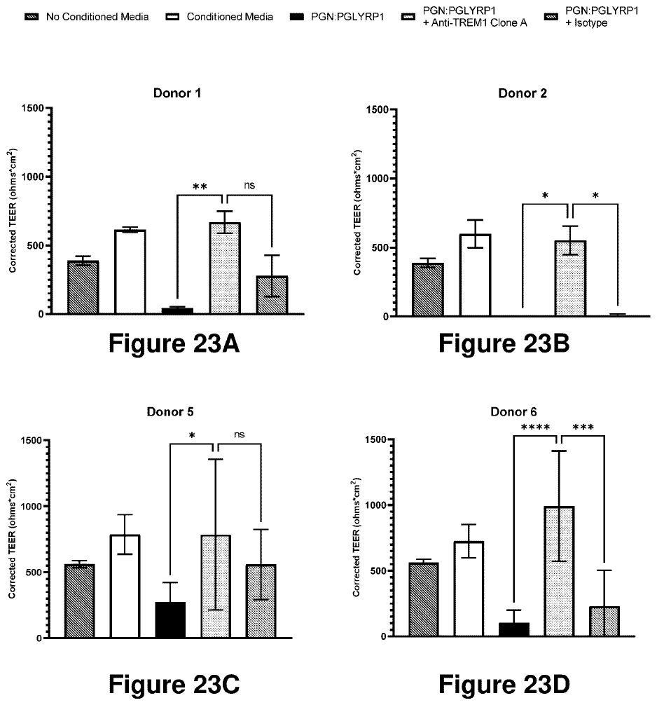

To determine whether TREM1 inhibition can prevent or reduce epithelial barrier damage caused by TREM1-mediated amplified inflammatory response, conditioned media was also produced from cultured monocytes stimulated with PGN (100 ng/mL) and PGLYRP1 (500 nM), and treated with 5 nM anti-TREM1 antibody agent clone A. As comparators, conditioned media was also produced from cultured monocytes that were not stimulated (conditioned media), stimulated with PGN and PGLYRP1, or stimulated with PGN and PGLYPR1 and treated with an isotype control antibody. A condition without conditioned media was also included as an additional control. TEER assay results showed that exposure of epithelial cells to TREM1 conditioned media produced in the presence of anti-TREM1 antibody agent clone A showed a substantial improvement in TEER relative to exposure to conditioned media produced by monocytes stimulated with PGN and PGLRYP1 alone, or in the presence of an isotype control antibody (FIGS. 23A-23D). These results demonstrate that TREM1 inhibition reduces or prevents epithelial barrier damage mediated by monocytes.

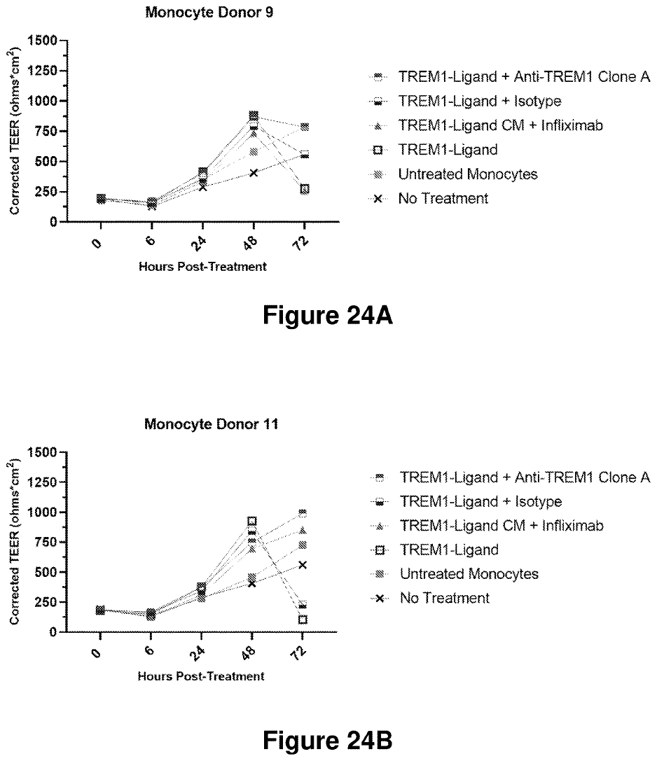

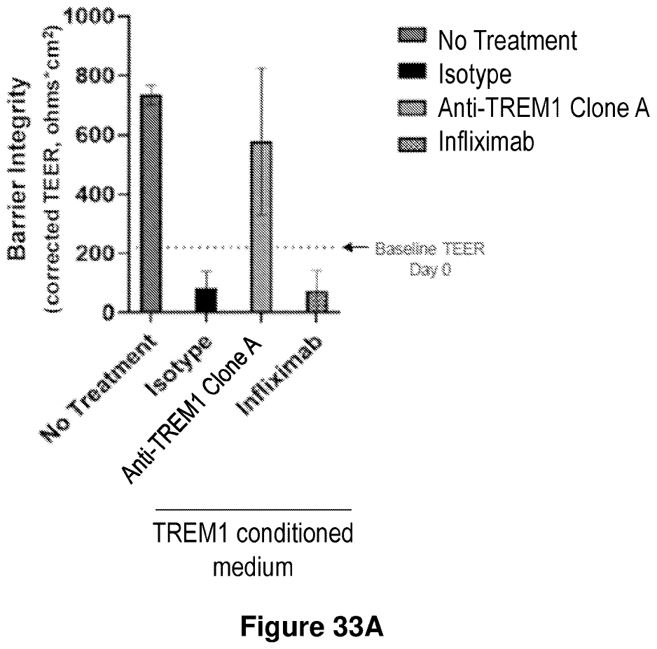

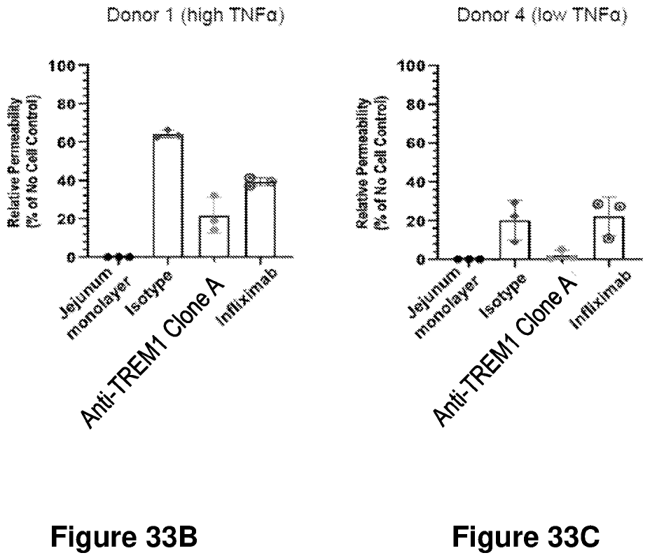

As conditioned media produced by stimulated monocytes contained varying levels of TNFα depending on the donor of the monocytes (Table 5), it was hypothesized that the efficacy of anti-TNFα antibody alone in preventing or reducing epithelial barrier damage may be donor-dependent and correlated with the level of TNFα produced by the stimulated monocytes. To test this hypothesis, conditioned media was produced using monocytes that were not stimulated (untreated monocytes), stimulated with PGN and PGLYRP1 (TREM1-Ligand), or PGN and PGLRYP1 in the presence of anti-TREM1 antibody agent clone A, an anti-TNFα antibody, or an isotype control. A condition without conditioned media (no treatment) was also included as an additional control. Conditioned media was produced using monocytes from different donors (Monocyte Donor 9 and 11) that showed differing levels of TNFα induction upon stimulation. Monocyte Donor 9 produced conditioned media with relatively lower levels of TNFα, while Monocyte Donor 11 produced conditioned media with relatively higher levels of TNFα. Jejunum cells were cultured in a monolayer and the basal compartment was exposed to the conditioned media. TEER assay results showed that conditioned media from monocytes stimulated with a combination PGN and PGLRYP1 alone or in the presence of an isotype control antibody showed an increase in epithelial barrier damage over time (FIGS. 24A-24B, and FIGS. 33A-33C). Conditioned media from monocytes stimulated in the presence of an anti-TNFα antibody caused less epithelial barrier damage as compared to isotype control when the conditioned media was produced from monocytes having high TNFα induction upon stimulation (FIG. 24B), but not when the conditioned media was produced from monocytes having low TNFα induction upon stimulation (FIG. 24A). In contrast, presence of anti-TREM1 antibody agent A when producing the conditioned media was effective in reducing epithelial barrier damage to a greater extent than presence of anti-TNFα antibody, regardless of differences in TNFα induction levels between different donors (FIGS. 24A-24B, and FIGS. 33A-33C). These results demonstrate that response to anti-TNFα antibody therapy may be subject dependent based on the levels of TNFα secreted by monocytes in response to stimulation. The results also demonstrate that TREM1 inhibition therapy can prevent epithelial barrier damage due to soluble mediators of TREM1 amplified inflammatory responses regardless of TNFα induction levels.

### Example 7: Mechanisms of TREM1 Inhibition by an Anti-TREM1 Antibody Agent

The present Example demonstrates that an anti-TREM1 antibody agent inhibits TREM1 by (i) blocking interaction between TREM1 and its ligand PGLYRP1, and (ii) by reducing the cell surface level of TREM1.

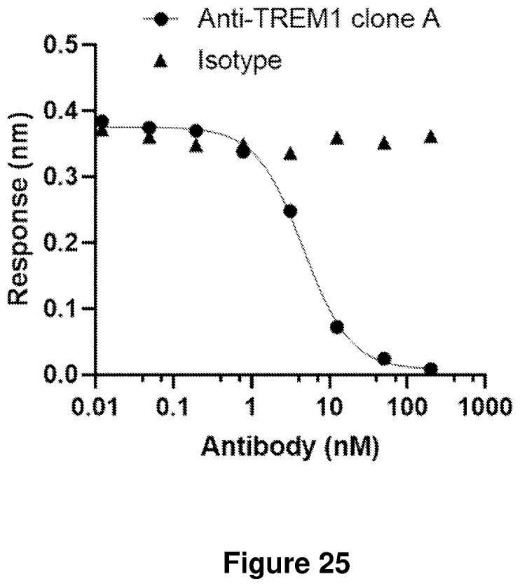

A bio-layer interferometry (BLI) assay was used to determine whether anti-TREM1 antibody agent clone A blocks interaction between TREM1 and PGLYRP1. Briefly, recombinant hTREM1-chFc1 was immobilized on Octet AHC biosensors and then blocked with 100 μg/mL hIgG1-Fc. The immobilized TREM1 protein was subsequently exposed to an 8-point titration from 30 to 0.0018 μg/mL (200 to 0.012 nM) of either anti-TREM1 clone A or isotype control for 300 s, followed by 20 μg/mL (971 nM) PGLYRP1. The incubation time between mAb and immobilized TREM1-ECD is limited to 300 s, thus limiting the amount of antibody that can bind to the immobilized TREM1, particularly at the sub-saturation concentrations. The results showed that the anti-TREM1 antibody agent was able to reduce the binding of TREM1 to PGLYRP1 in a dose-dependent manner, while isotype control antibody did not inhibit TREM1-PGLYRP1 interaction (FIG. 25). Anti-TREM1 clone A blocks PGLYRP1 binding to TREM1-ECD with an IC50 of 4.5+0.29 nM, in this assay format. This result demonstrates that the anti-TREM1 antibody agent inhibits TREM1 by blocking TREM1 binding to PGLYRP1.

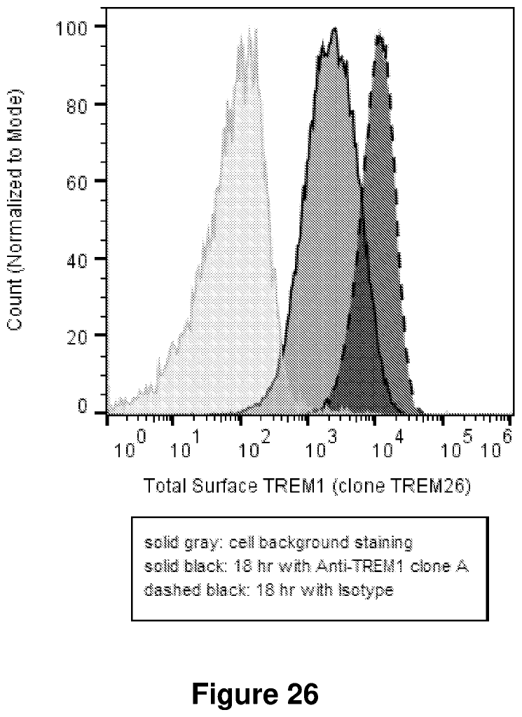

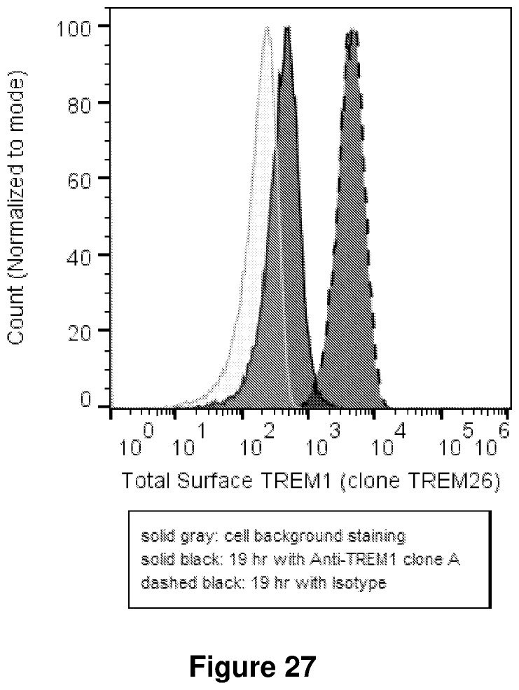

A cell culture assay was used to determine whether the anti-TREM1 antibody agent reduces levels of cell surface TREM1. Briefly, isolated human peripheral CD14⁺ monocytes were cultured in the presence of 5 μg/mL anti-TREM1 antibody agent clone A (33 nM) or isotype control (33 nM). After about 18-19 hours, cell surface TREM1 levels were assessed by staining with a PE-conjugated anti-TREM1 antibody (BioLegend, TREM26) that did not shared the same epitope as anti-TREM1 clone A. The stained cells were analyzed by flow cytometry. The results demonstrated that treatment with anti-TREM1 clone A reduced the levels of TREM1 on the cell surface of monocytes (FIG. 26). A similar experiment using isolated human peripheral neutrophils also showed reduced levels of TREM1 on the cell surface of neutrophils after treatment with anti-TREM1 clone A (FIG. 27). Without wishing to be bound by any particular theory, we hypothesize that reduced cell surface TREM1 levels may be due to internalization of TREM1.

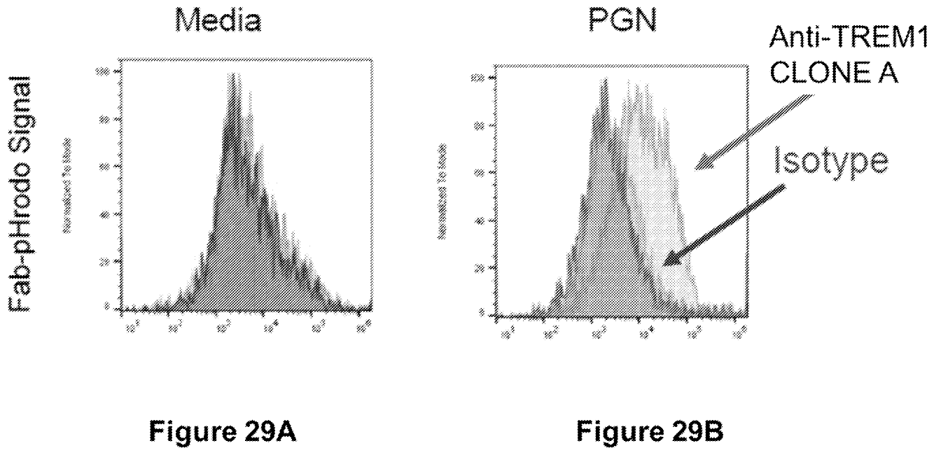

In another cell-based assay, human monocytes isolated from healthy volunteers were treated with peptidoglycan (PGN; 1 μg/ml) to stimulate increased TREM1 expression on the cell surface (FIG. 29B) or treated with media alone (FIG. 29A). Cells were then treated with anti-TREM1 antibody agent clone A or isotype control, then incubated with an anti-hIgG1 Fab labeled with a pH-sensitive dye. As measured by flow cytometry analysis, the increase in the pH-dependent signal shown in FIG. 29B indicates that surface TREM1 was internalized into acidic late endosomes/lysosomes in the presence of anti-TREM1 antibody agent clone A, but not isotype control. Accordingly, these data suggest that binding of anti-TREM1 antibody agent clone A to TREM1 on the cell surface, promotes internalization of TREM1.

### Example 8: Anti-TREM1 Restores Differentiation of Monocytes to Macrophages

The present Example demonstrates that an anti-TREM1 antibody agent restores differentiation of monocytes into macrophages.

Without wishing to be bound by any particular theory, it is thought that circulating monocytes will form a limited, transient population of inflammatory monocytes when infiltrating healthy tissues, in which inflammatory monocyte population rapidly matures to macrophages that secrete tolerogenic factors and facilitate maintenance of epithelial barrier integrity. In contrast, it is thought that in diseased tissues, inflammatory signals arrest monocytes as inflammatory monocytes inhibiting their differentiation into macrophages. In this condition, the low abundance of macrophages reduces anti-inflammatory signaling, while the accumulated inflammatory monocytes secrete proinflammatory factors (e.g., TNFα). Without wishing to be bound by any particular theory, we note that most anti-inflammatory therapies target effectors that are downstream of inflammatory monocytes (e.g., TNFα), but do not address the increased levels of inflammatory monocytes and decreased levels of tolerogenic macrophages that occur in diseased tissue.

To determine whether TREM1 inhibition can antagonize the inhibition of maturation of monocytes to macrophage, monocytes were differentiated in vitro in the presence of a combination of bacterial peptidoglycan (PGN) and PGLYRP1, alone or in the presence of either anti-TREM1 antibody agent clone A or an isotype control antibody. Briefly, primary human CD14+ monocytes were isolated from peripheral blood of healthy volunteers. Monocytes were cultured and were exposed to inflammatory cytokine cocktail of GM-CSF (2.5 ng/mL), IL-23 (10 ng/mL), and IFN-γ (50 ng/mL) for 6 days to generate inflammatory monocytes. Inflammatory monocytes were then cultured in the presence of a macrophage promoting cytokine cocktail comprising TGF-β (1 ng/mL) and IL-10 (10 ng/mL) to induce differentiation toward macrophages. Addition of PGN (1 μg/mL) and PGLYRP1 (250 nM) were used to test the effect of TREM1 activation on inhibiting maturation of the inflammatory monocytes to macrophages. Further addition of 10 nM of either anti-TREM1 antibody agent clone A or an isotype control antibody was used to assess the ability of TREM1 inhibition to restore inflammatory monocyte differentiation to macrophages. The cells were cultured for an additional six days in the presence of the macrophage promoting cytokine cocktail, followed by staining for cell surface markers CD64 and MERTK, and analysis by flow cytometry.

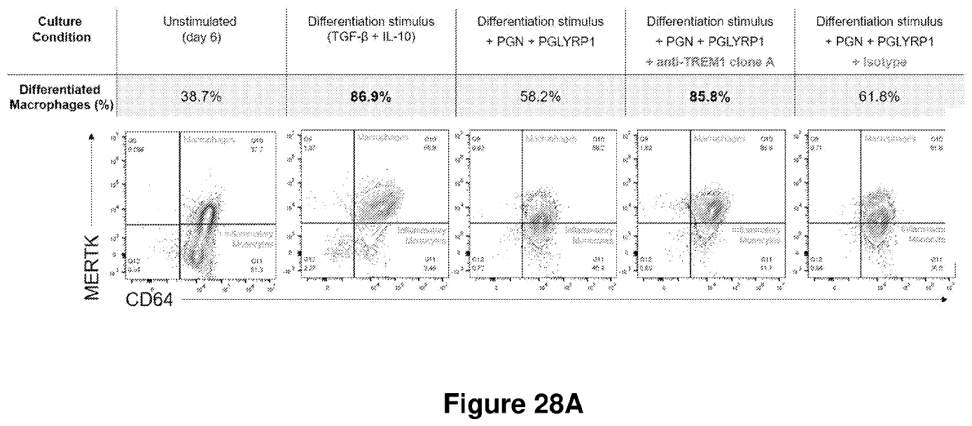

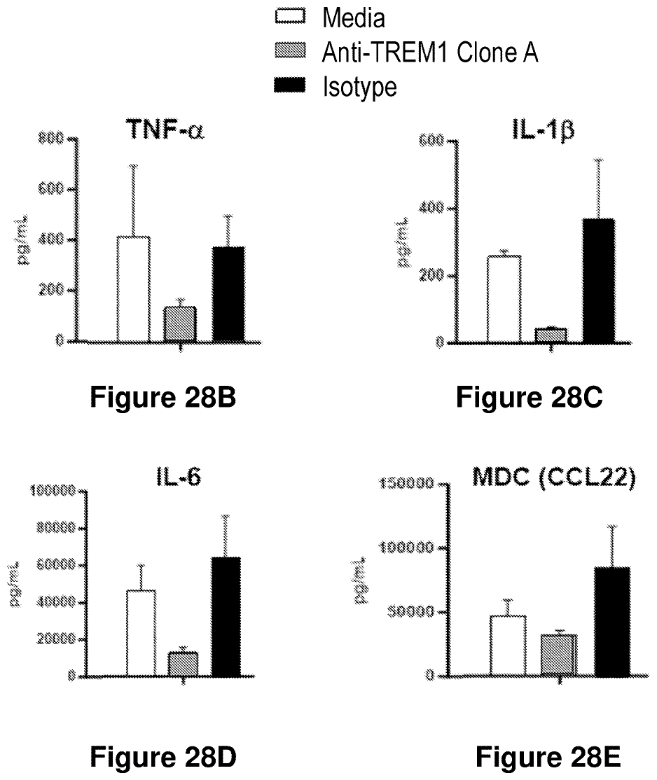

The results show that when inflammatory monocytes are cultured in the presence of the macrophage promoting cytokine cocktail (differentiation stimulus), the cells show an increase in macrophages (CD64+MERTK+) and a decrease in inflammatory monocytes (CD64+MERTK−) relative to unstimulated inflammatory monocytes (unstimulated) (FIG. 28). Addition of PGN and PGLYRP1 to the differentiation media reduced the percentage of observed differentiated macrophages (FIG. 28), indicating that TREM1 activation inhibits differentiation of inflammatory monocytes toward macrophages. Further addition of anti-TREM1 antibody agent clone A showed increased levels of differentiated macrophages, while an isotype control antibody did not impact differentiation (FIG. 28A), demonstrating that TREM1 inhibition can antagonize TREM1-mediated inhibition of maturation of inflammatory monocytes to macrophages, resulting in fewer inflammatory monocytes and increased levels of mature macrophages. These data suggest that anti-TREM1 antibody agent clone A promotes the increase in the number of tolerogenic macrophages. FIGS. 28B-28E similarly show that anti-TREM1 antibody agent clone A-mediated increase in tolerogenic macrophages is functionally reflected by reduced production of inflammatory mediators (e.g., TNF-α, IL-1, IL-6, and MDC (CCL2)).

### Example 9: Epitope Mapping

This example describes experiments characterizing the epitope of TREM1 bound by anti-TREM1 antibody agent clone A using deuterium exchange (HDX) mass spectrometry (MS) analysis.

A solution of TREM1 protein (10 μM, 150 μl) was introduced in an HDX robotic sample handling system (Waters). The pipetting robot mixed 4 μl of the protein sample with 56 1 of labeling buffer (5 mM K2HPO4, 5 mM KH2PO4, D2O pH 6.6) for exchange analysis or equilibration buffer (5 mM K2HPO4; 5 mM KH2PO4, pH7.0) for control analysis. The 15× diluted solution (60 pl, 0.66 pM) was incubated within the robot (22° C.) for 15s, 60s, 180s, 600s, 1800s and 7200 seconds. After incubation, 50 μl of the quench solution (50 mM K2HPO4; 50 mM KH2PO4; GuCl 2.0 M, TCEP 200 mM, pH 2.3, 0° C.) were added to the sample. After incubation, 80 μl of the quenched protein solution were immediately injected into the proteolytic pepsin column for 3 minutes at 15° C. After proteolysis, the pepsin peptides generated were submitted to C18 chromatography (HDX Manager Waters) for 420 seconds at 0° C. before MSe Xevo-G2-XS analysis. These experiments have been repeated in triplicates.

A 140 μl solution of TREM1 protein mixed with Anti-TREM1 antibody agent clone A was prepared with a 10 pM:20 pM final concentration ratio. The protein solution was then incubated within the sample handling robot at 22° C. for 90 minutes. After incubation, the pipetting robot mixed 4 μl of the protein sample with 56 μl of Labeling buffer (5 mM K2HPO4; 5 mM KH2PO4, D20 pH 6.6) for exchange experiment or equilibration buffer (5 mM K2HPO4; 5 mM KH2PO4, pH7.0) for control experiment. The 15× diluted solution (60 pl, PR_402:AB_383, PR_402:AB_377 or PR_402:AB_373; 0.66 M:1.33 M) was incubated within the robot (22° C.) for 15s, 60s, 180s, 600s, 1800s and 7200 seconds. After incubation, 50 μl of the quench solution (50 mM K2HPO4, 50 mM KH2PO4; GuCl 2.0 M, TCEP 200 mM, pH 2.3, 0° C.) were added to the sample for 20s incubation time. After incubation, 80 μl of the quenched protein solution were immediately injected into the proteolytic pepsin column for 3 minutes at 15° C. After proteolysis, the pepsin peptides generated were submitted to C18 chromatography (HDX Manager Waters) for 420 seconds at 0° C. before MSe Xevo-G2-XS analysis. Experiments were performed in triplicate.

### Liquid Chromatography

After proteolysis, 80 μl of the peptide solution generated by proteolysis were loaded onto liquid chromatography system. (HDX Manager Waters) using the parameters described in Table 9.

TABLE 9
Liquid Chromatography Parameters

| | |
|---|---|
| Buffer A | 100/0.2/H2O/HCOOH (v/v) |
| Buffer B | 5/95/0.2 H2O/ACN/HCOOH (v/v/v) |
| Gradient | 5 at 40% B in 7 minutes |
| Injected Volume | 80 μl |
| Precolumn | Acquity UPLC BEH C18 Vanguard Pre- |
| | Column, 1.7 μm, 2.1 mm*5 mm |
| Precolumn flow rate | 40 μl/min |
| Column | Acquity IPLC BEH C18 1.7 μm, |
| | 1 mm*100 mm |
| Column flow rate | 40 μl/min |

The Xevo-G2-XS MSe analysis has been performed the parameters described in Table 10.

TABLE 10
Mass Spectrometry Parameters

| Ionization mode | ESI+ | |
|---|---|---|
| Acquisition mode | Sentivity | |
| Acquisition range | 50-2000 | m/z |
| Capillary voltage | 2.8 | kV |
| Sampling cone | 30 | V |
| Source offset | 40 | V |
| Source temperature | 80° | C. |
| Desolvation temperature | 250° | C. |
| Cone gas | 20 | L/h |
| Desolvation gas | 800 | L/h |
| Acquire lockspray | Acquire but not apply correction | |
| Lockspray Reference | Leucine Enkaphalin single point MS | |
| | (556.2771 Da) | |
| Lockspray scan time | 0.5 | sec |
| Interval | 60 | sec |

### Data Processing

Data processing was performed using the ProteinLynx global server (PLGS) according to the parameters listed in Table 11.

TABLE 11
PLGS Data Processing Parameters

| Chromatographic Peak Width | Automatic | |
|---|---|---|
| MS ToF Resolution | Automatic | |
| Lock Mass Charge 1 | 556.2771 | Da |
| Lock Mass Window | 0.25 | Da |
| Low Energy Threshold | 500 | counts |
| Elevated Energy Threshold | 100 | counts |
| Elution Start Time | 2 | min. |
| Elution End Time | 9.5 | min. |

| Enzyme | Pepsin |
|---|---|
| Missed cleavages | 1 |
| False Discovery Rate | 100 |
| Peptide Tolerance | Automatic |
| Fragment Tolerance | Automatic |

### Results

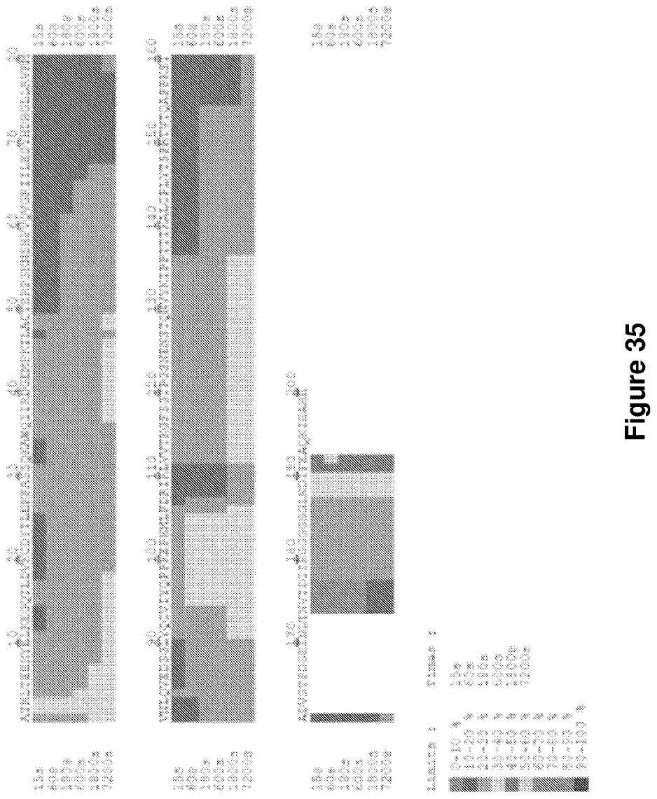

For the H/D experiments of TREM1 mixed with Anti-TREM1 antibody agent clone A, 58 peptides were identified both in control and exchange experiments after the incubation times 15s, 60s, 180s, 600s, 1800s and 7200s. For each incubation time, the % of incorporation of deuterium was determined based on average values of triplicate experiments. The determination of the deuterium incorporation in each pepsin peptide of TREM1 mixed with Anti-TREM1 antibody agent clone A for each incubation time resulted in the deuterium exchange heat map shown in FIG. 35.

When mixing with Anti-TREM1 antibody agent clone A, 10%-15% of incorporation difference was observed between positions 174 and 190 (NVTDIIRGGGGSGLNDI). 5%-10% of incorporation difference was observed between positions 18 and 25 (DVKCDYTL).

### Example 10: Pharmacokinetic/Toxicity Analysis in Cynomolgus Monkeys

This example describes experiments analyzing the pharmacokinetic (PK) profile of Anti-TREM1 antibody agent clone A. The PK of anti-TREM1 antibody agent clone A was evaluated in 4 studies in cynomolgus monkeys: (i) a single-dose non-good laboratory practice (GLP) PK study; (ii) a repeated-dose non-GLP PK study with 4 weeks of weekly dosing; (iii) a 14-week GLP toxicity study; and (iv) a 26-week GLP toxicity study.

### Methods—Pharmacokinetics

MSD® plates coated with the working solution of anti-TREM1 antibody agent were utilized. Prior to sample plating, the plate was incubated and washed and then blocked, incubated, and then washed again. Standards, quality controls, and dosed cynomolgus monkey serum samples were added to the appropriate wells of the coated plate, and the plate was incubated and washed. The primary anti-TREM1 detection working solution was added to the wells, and the plate was incubated and washed. The secondary detection solution was then added to the wells, and the plate was incubated and washed. Finally, 2× Read Buffer T was added to the wells. The plates were read within 10 minutes of the addition of 2× Read Buffer T.

### Methods—Antibody Drug Antibodies

MSD® MULTI-ARRAY® Gold 96-well streptavidin-coated plates were utilized. Prior to sample plating, acetic acid was added to an acid dissociation dilution storage plate. Controls and dosed cynomolgus monkey serum samples were added to the appropriate wells of the acid dissociation dilution storage plate, and the plate was incubated. Anti-TREM1 antibody Mastermix [Anti-TREM1 antibody-biotin+AntiTREM1 antibody-SULFO-TAG+neutralizing buffer (375 mm Tris-HCL, 2% BSA in 2× Tris-buffered saline)]was added to the appropriate wells of a neutralizing Mastermix storage plate, the samples were transferred from the acid dissociation dilution storage plate to the neutralizing Mastermix storage plate, and the Mastermix storage plate was incubated. The incubated samples were added to duplicate wells of a streptavidin-coated plate, and the plate was incubated and washed. Finally, 2× Read Buffer T was added to the wells. The plates were read within 10 minutes of the addition of 2× Read Buffer T.

### Methods—Receptor Occupancy

Whole blood samples were analyzed for target monocytes and neutrophils identified according to the antigenic markers described in Table 12.

TABLE 12
Cell Population Antigenic Markers

| Antigen Marker | Cell Population |
|---|---|
| CD45+/CD66abce+ | Neutrophils |
| CD45+/CD14+ | Monocytes |
| CD45+/CD66abce+/Anti-TREM1 | Anti-TREM1 antibody agent |
| antibody agent clone A+ | clone A bound to neutrophils |
| CD45+/CD14+/Anti-TREM1 | Anti-TREM1 antibody agent |
| antibody agent clone A+ | clone A bound to monocytes |
| CD45+/CD14+/TREM1+ | Total monocyte receptors |
| CD45+/CD66abce+/TREM1+ | Total neutrophil receptors |

Antibodies used for flow cytometry were as described in Table 13.

TABLE 13
Receptor Occupancy Flow Cytometry Antibodies

| Fluorophore | FITC | PE | AF647 | Vioblue | BC510 |
|---|---|---|---|---|---|
| Panel | CD45 | TREM1 | Anti-TREM1 | CD66abce | CD14 |
| | | | antibody | | |
| | | | agent clone A | | |
| Isotype | CD45 | Mouse IgG1, | Pali-hIgH1- | CD66abce | CD14 |
| Panel | | κ Isotype | PVAdG- | | |
| | | Control-PE | AF647 | | |

The dosed cynomolgus monkey serum samples were collected for evaluation of monocytes and neutrophils by flow cytometry. The samples were accessioned and processed on the day of collection. The flow cytometry data for the cell populations were reported as a percentage of total leukocytes for neutrophil and monocyte populations. Receptor occupancy data for total and free receptor occupancy were reported as median fluorescent intensity (MFI) and percent of baseline.

### Results

Across all PK studies in cynomolgus monkeys, 0/6 animals, 0/12 animals, 1/22 animals, 1/31 animals receiving anti-TREM1 antibody agent clone A developed anti-TREM1 antibody agent clone A antidrug antibodies (ADA) in the (i) a single-dose non-good laboratory practice (GLP) PK study; (ii) a repeated-dose non-GLP PK study with 4 weeks of weekly dosing; (iii) a 14-week GLP toxicity study; and (iv) a 26-week GLP toxicity study, respectively. In the 2 animals with ADA, the ADA were not associated with any safety findings. However, anti-TREM1 antibody agent clone A exposure was not detected in the animal in the 14-week GLP toxicity study and was reduced, but detected throughout the dosing interval, in the animal in the 26-week GLP toxicity study.

The PK of anti-TREM1 antibody agent clone A in cynomolgus monkeys was characterized by a terminal elimination half-life (t_{1/2}) of approximately 7 days. The mean maximum concentration (Cₘₐₓ) values of anti-TREM1 antibody agent clone A increased with increasing dose in an approximately dose-proportional manner, and the mean area under the concentration versus time curve from 0 to 168 hrs. postdose (AUC₀₋₁₆₈) increased with increasing dose in a less than dose-proportional manner. Systemic exposure to anti-TREM1 antibody agent clone A, based on AUC, did not consistently change (generally <2-fold) with repeated administration of anti-TREM1 antibody agent clone A.

### 10.1 Single-Dose Studies

The PK of anti-TREM1 antibody agent clone A following a single IV bolus administration at 1, 3, or 10 mg/kg to cynomolgus monkeys (2 males per group) was characterized in a non-GLP study. Concentrations of anti-TREM1 antibody agent clone A were determined in serum at multiple timepoints through 648 hours postdose using a developed electrochemiluminescent (ECL) ligand-binding assay. The presence of anti-TREM1 antibody agent clone A ADAs was assessed predose and at 312 and 648 hours postdose using a developed ECL bridging assay. Whole blood was collected for the determination of total and free TREM1 on neutrophils using a flow cytometry based RO assay over the 648 hour time course.

All samples either screened negative or had negative titer results when evaluated for the presence of anti-TREM1 antibody agent clone A ADAs.

The PK of anti-TREM1 antibody agent clone A following a single IV dose is summarized in Table 14.

TABLE 14
Mean PK Parameters in Monkey Serum Following Single IV
Administration of Anti-TREM1 Antibody Agent Clone A

| | | |
|---|---|---|
| Dose (mg/kg) | 3 | 10 |
| Sex/Group Size | M/2 | M/2 |
| Cₘₐₓ (μg/ml) | 95.5 | 249 |
| Cₘₐₓ/dose (μg/ml)/(mg/kg) | 31.8 | 24.9 |
| tₘₐₓ (h) | 1.0 | 1.5 |
| AUC₀₋₁₆₈ (μg × h/ml) | 4000 | 10200 |
| AUC₀₋₁₆₈/dose (μg × | 1330 | 1020 |
| h/ml)/(mg/kg) | | |
| AUCₗₐₛₜ (μg × h/ml) | 6610 | 15300 |
| AUC_{0-inf} (μg × h/ml) | 7290 | 15700 |
| t_{1/2} (h) | 174 | 126 |
| CL (ml/h/kg) | 0.461 | 0.654 |
| Vₛₛ (mg/kg) | 88.6 | 104 |

AUC₀₋₁₆₈ = area under the concentration vs time curve (AUC) from 0 to 168 hours postdose; AUC_{0-inf =} AUC from 0 to infinity; AUC₀₋ₗₐₛₜ = AUC from 0 to the last quantifiable concentration; CL = clearance; Cₘₐₓ = maximum concentration; M = male; t_{1/2} = terminal elimination half-life; tₘₐₓ = time to maximum concentration; Vₛₛ = volume of distribution at steady state.

Receptor occupancy (RO) on neutrophils was also assessed. Across all doses, full RO (<10% of baseline free TREM1) was observed on neutrophils as early as 1 hour after dosing. This level of RO was maintained as long as there was a measurable level of anti-TREM1 antibody agent clone A in the serum. In addition, after dosing, an approximate 50% decrease from baseline of total TREM1 levels on neutrophils was observed throughout the end of the study. The decrease in total TREM1 was consistent with the pharmacologic activity of anti-TREM1 antibody agent clone A as a component of the of anti-TREM1 antibody agent clone A mechanism of action is to remove TREM1 from the cell surface.

### 10.2 Repeated-Dose Studies

The PK of free anti-TREM1 antibody agent clone A was determined in a non-GLP study following once-weekly IV bolus administration to cynomolgus monkeys (4 males per group) at 3, 20, or 50 μg/kg for 4 weeks. Concentrations of anti-TREM1 antibody agent clone A were determined in serum at multiple timepoints through 648 hours postdose using a developed electrochemiluminescent (ECL) ligand-binding assay on Day 1 and Day 22 up to 144 hours and 168 hours postdose, respectively, and on Day 8 and Day 15 predose. The presence of anti-TREM1 antibody agent clone A ADAs was assessed in serum samples collected prior to dosing on Day 1 and Day 22 and on Day 14 and Day 28 postdose using a developed ECL bridging assay. Whole blood was collected on Day 1 and Day 22 over 96 and 168 hour time courses, respectively, and on Day 8 and Day 15 predose for the determination of total and free TREM1 on neutrophils using a flow cytometry based RO assay.

Serum concentration-profiles of anti-TREM1 antibody agent clone A were characterized by a short distribution phase of approximately 2 days followed by a longer terminal elimination phase. Both Cₘₐₓ and AUC increased with increasing dose. The increase in Cₘₐₓ was approximately dose proportional for the first dose but was less than dose proportional on Day 22 at the highest dose level of 50 mg/kg. On Day 1 and Day 22, the increase in AUC₀₋₁₆₈ was less than dose proportional at all dose levels tested. The t_{1/2} on Day 1 ranged from 3.6 to 4 days (85.8 to 95.4 hours).

Clearance increased with dose for all 3 dose levels on Day 1 and Day 22. Based on AUC₀₋₁₆₈, the accumulation was modest (ratios were <1.5 at all dose levels). The Cₘₐₓ was broadly similar on Day 1 to Day 22

All samples were negative for the presence of anti-TREM1 antibody agent clone A ADAs.

The PK of anti-TREM1 antibody agent clone A following single and repeated IV doses is summarized in Table 15.

TABLE 15
Mean PK Parameters in Monkey Serum Following Weekly IV Administration
of Anti-TREM1 Antibody Agent Clone A for 4 weeks

| | Dose (mg/kg) | | | | | |
|---|---|---|---|---|---|---|
| | 3 | | 10 | | 50 | |
| | Sex/Group Size | | | | | |
| | M/4 | | M/4 | | M/4 | |
| | Day | | | | | |
| | 1 | 22 | 1 | 22 | 1 | 22 |
| Cₘₐₓ (μg/ml) | 104 | 87.9 | 701 | 699 | 1780 | 986 |
| Cₘₐₓ/dose | 34.6 | 29.3 | 35.1 | 34.9 | 35.6 | 19.7 |
| (μg/ml)/(mg/kg) | | | | | | |
| AUC₀₋₁₆₈ (μg × | 4500 | 5940 | 17600 | 18900 | 28000 | 38300 |
| h/ml) | | | | | | |
| AUC₀₋₁₆₈/dose (μg × | 1520 | 1980 | 880 | 944 | 560 | 767 |
| h/ml)/(mg/kg) | | | | | | |
| t_{1/2} (h) | 85.8 | 279 | 86.7 | 133 | 95.4 | 135 |
| CL (ml/h/kg) | 0.667 | 0.511 | 1.15 | 1.09 | 1.8 | 1.34 |

AUC₀₋₁₆₈ = area under the concentration vs time curve (AUC) from 0 to 168 hours postdose;
CL = clearance;
Cₘₐₓ = maximum concentration;
M = male;
t_{1/2} = terminal elimination half life

At the end of the recovery period (Day 140, 1 out of 4 animals in the 50 mg/kg group no longer had detectable concentrations of anti-TREM1 antibody agent clone A in serum, and the free TREM1 receptors on neutrophils in this animal demonstrated partial recovery. Total TREM1 remained decreased in all 4 animals in the 50 mg/kg group at the end of recovery.

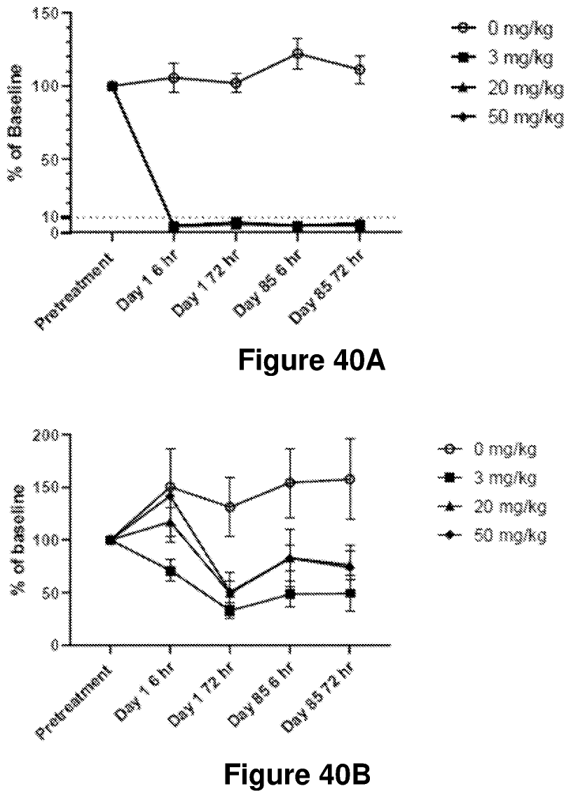

Receptor occupancy (RO) on neutrophils was also assessed. After dosing on Day 1 and Day 85, all dose groups demonstrated full RO, which was defined as <10% of baseline free TREM1, 6 and 72 hours after dosing (FIG. 40A). This level of RO was maintained throughout the study. In addition, all dose groups demonstrated a significant decrease in total TREM1 72 hours after dosing on Day 1 and 6 and 72 hours after dosing on Day 85 (FIG. 40B). This reduction in total TREM1 was consistent with the pharmacologic activity of anti-TREM1 antibody agent clone A as a component of its mechanism of action to remove TREM1f-om the cell surface.

### 10.3 14-Week Anti-TREM1 Antibody Agent Clone A GLP Toxicity Study

This study describes experiments to evaluate the potential toxicity of anti-TREM1 antibody agent clone A, when administered by intravenous bolus injection once weekly for 14 weeks to cynomolgus monkeys and to evaluate the potential reversibility of any findings. In addition, the toxicokinetic characteristics anti-TREM1 antibody agent clone A were determined.

Group treatments were performed as described in Table 16.

TABLE 16
Group Treatments for 14 week Toxicology Analysis

| | | | Dose | Dose | # Male | # Female | # Male | # Female |
|---|---|---|---|---|---|---|---|---|
| | Test | Dose | Volume | Concentration | Cynos | Cynos | Cynos | Cynos |
| Group | Article | (mg/kg) | (mL/kg) | (mg/ml) | (Main) | (Main) | (Recovery) | (Recovery) |
| 1 | Control | 0 | 2 | 0 | 3 | 3 | 2 | 2 |
| 2 | Anti- | 3 | 2 | 1.5 | 3 | 3 | 0 | 0 |
| | TREM1 | | | | | | | |
| | Antibody | | | | | | | |
| | Agent | | | | | | | |
| | clone A | | | | | | | |
| 3 | Anti- | 20 | 2 | 10 | 3 | 3 | 0 | 0 |
| | TREM1 | | | | | | | |
| | Antibody | | | | | | | |
| | Agent | | | | | | | |
| | clone A | | | | | | | |
| 4 | Anti- | 50 | 2 | 25 | 3 | 3 | 2 | 2 |
| | TREM1 | | | | | | | |
| | Antibody | | | | | | | |
| | Agent | | | | | | | |
| | clone A | | | | | | | |

The following parameters and endpoints were evaluated in this example: mortality, clinical observations, injection site observations/dermal scoring, sex differences, body weights, qualitative food consumption, ophthalmology, electrocardiology exams, respiratory rate (visual), neurologic examination, clinical pathology parameters (hematology, coagulation, clinical chemistry, and urinalysis values), bioanalysis, immunophenotyping, organ weights, and macroscopic and microscopic examinations. For all these assessed parameters, no statistically relevant perturbations were observed following once weekly IV administration of anti-TREM1 antibody agent clone A for 14 weeks. The presence of anti-drug antibodies, cytokine parameters, and receptor occupancy were also assessed in dosed animals and further described below.

Consistent with the anticipated pharmacological activity of anti-TREM1 antibody agent clone A, administration of 3, 20, or 50 mg/kg/dose anti-TREM1 antibody agent clone A resulted in significant decreases in the free TREM1 receptors on neutrophils for the dosing phase time points collected, Day 1 at 6 and 72 hours postdose and Day 85 at 6 and 72 hours postdose. At the end of the recovery period, 1 out of 4 animals in the 50 mg/kg/dose group did not have detectable concentrations of anti-TREM1 antibody agent clone A in serum and the free TREM1 receptors on neutrophils in this animal demonstrated partial recovery.

Following once weekly intravenous slow bolus injection of anti-TREM1 antibody agent clone A to monkeys, mean C₀ and Cₘₐₓ values of anti-TREM1 antibody agent clone A increased with increasing dose in an approximate dose proportional manner, and mean AUC₀₋₁₆₈ₕᵣ increased in a less than dose proportional manner on Days 1 and 85. Systemic exposure (AUC₀₋₁₆₈ₕᵣ values) to anti-TREM1 antibody agent clone A in monkeys did not appear to consistently change following repeated administration of anti-TREM1 antibody agent clone A. One animal in the low dose group was positive for anti-anti-TREM1 antibody agent clone A antibodies on Days 57 and 85. In this animal, systemic exposure (C₀, Cₘₐₓ, and AUC₀₋₁₆₈ₕᵣ values) to anti-TREM1 antibody agent clone A on Day 85 appeared to be impacted by the presence of anti-TREM1 antibody agent clone A ADAs

In conclusion, administration of anti-TREM1 antibody agent clone A by intravenous slow bolus injection once every week for 14 weeks was well tolerated in cynomolgus monkeys up to the level of the 50 mg/kg/dose, and there were no noteworthy toxicological findings observed. Based on the results under the conditions of this experiment, the no-observed-adverse-effect level (NOAEL) was considered to be 50 mg/kg/dose. At all dose levels of anti-TREM1 antibody agent clone A, there was a significant reduction in the free TREM1 receptors on neutrophils, consistent with the anticipated pharmacological action of anti-TREM1 antibody agent clone A. At the NOAEL, the gender combined mean Cₘₐₓ and AUC₀₋₁₆₈ₕᵣ values following Day 85 administration were 1440 μg/mL and 48500 μg·hr/mL.

All dose formulation results from Day 1 and Day 85 were acceptable and within acceptance criteria (Mean sample concentration of ±10% of theoretical concentration and Individual sample concentration of ±15%).

### Anti-Drug Antibody (ADAs)

The presence of anti-TREM1 antibody agent clone A anti-drug antibodies (ADAs) was also evaluated for cynomolgus monkeys administered with anti-TREM1 antibody agent clone A by IV slow bolus injection once every week for 14 weeks using a validated method.

Following screening analysis, two samples (Group 2 Female, 3 mg/kg/dose; Day 57 and Day 85) returned a potentially positive result. Those two samples proceeded to confirmatory analysis. Two samples confirmed positive and proceeded to titer analysis. The titer results were 15.1 (Day 57) and 62.4 (Day 85). All other samples screened negative.

Anti-TREM1 antibody agent clone A ADAs were not detected in any other animals, regardless of dose groups.

A pre-dose sample was taken prior to dose administration on Day 57. Anti-TREM1 antibody agent clone A could be detected in all animals at all dose levels pre-dose on Day 57 with the exception of a female animal(3 mg/kg/dose). A similar observation was made for the pre-dose samples collected on Day 85. Anti-TREM1 antibody agent clone A ADAs could be detected in this animal prior to dose administration on Days 57 and 85.

Additionally, a serum sample was collected at necropsy (approximately 120 hours after administration of the last dose on Day 92). anti-TREM1 antibody agent clone A could be detected in all animals at all dose levels with the exception of the female (3 mg/kg/dose) animal.

In the female (3 mg/kg/dose) animal, systemic exposure (Day 85 C₀, Cₘₐₓ, and AUC₀₋₁₆₈ₕᵣ values, pre-dose concentrations on Days 57 and 85, and at necropsy) to anti-TREM1 antibody agent clone A appeared to be impacted by the presence of anti-TREM1 antibody agent clone A ADAs.

### Receptor Occupancy

Administration of 3, 20, or 50 mg/kg/dose anti-TREM1 antibody agent clone A resulted in significant decreases across all 3 dose groups in the free TREM1 receptors on neutrophils on Day 1 at the 6 and 72 hour timepoints and on Day 85 at the 6 and 72 hour timepoints. In addition, there were significant decreases across all 3 dose groups in the total TREM1 receptors on both Day 1 (72 hr timepoint) and Day 85 (6 and 72 hr timepoints), by staining with a non-anti-TREM1 antibody agent clone A competing anti-TREM1 antibody CD354 PE (clone: TREM26). At the end of the recovery period (Day 140), 1 out of 4 animals in the 50 mg/kg/dose group did not have detectable concentrations of anti-TREM1 antibody agent clone A in serum (<400 ng/mL) and the free TREM1 receptors on neutrophils in this animal demonstrated partial recovery. Total TREM1 receptors remained decreased in all 4 animals in the 50 mg/kg/dose group at the end of recovery (Day 140).

### Cytokine Analysis

There were no changes in plasma IL-2, IL-8, MCP-1, or IL-IRA concentrations that were attributed to anti-TREM1 antibody agent clone A administration. Plasma IL-2, IL-8, MCP-1, and IL-1RA concentrations were generally below the LLOQ at all time points evaluated or were within the range of predose and/or control values.

There were no changes in the plasma IL-1β, IL-5, IL-6, IL-12/23 (p40), IL-13, IL-17, G-CSF, GM CSF, IFN-γ, TNF-α, IL-1β, or MIP-1β concentrations that were attributed to anti-TREM1 antibody agent clone A administration at any time point evaluated in comparison to the respective prestudy and/or control values. Plasma IL-1β, IL-5, IL-6, IL-12/23 (p40), IL-13, IL-17, G-CSF, GM CSF, IFN-γ, TNF-α, IL-1β, and MIP-1β concentrations were either <lower limit of quantitation (LLOQ) or within the range of pre-dose and/or control values at all time points for all dosed animals.

### 10.4 26 Week Anti-TREM1 Antibody Agent Clone A GLP Toxicity Study

This study describes experiments to evaluate the potential toxicity of Anti-TREM1 antibody agent clone A, when administered by intravenous bolus injection once weekly for 26 weeks to cynomolgus monkeys and to evaluate the potential reversibility of any findings. In addition, the toxicokinetic characteristics anti-TREM1 antibody agent clone A were determined.

Group treatments were performed as described in Table 17.

TABLE 17
Group Treatments for 26 week Toxicology Analysis

| | | | | Dose | Dose | # Male | # Female | # Male | # Female |
|---|---|---|---|---|---|---|---|---|---|
| | Test | Dose | | Volume | Concentration | Cynos | Cynos | Cynos | Cynos |
| Group | Article | (mg/kg) | Route | (mL/kg) | (mg/ml) | (Main) | (Main) | Recovery | Recovery |
| 1 | Control | 0 | SC/IV | 0.4/2 | 0 | 3 | 3 | 3 | 3 |
| 2 | Anti- | 3 | IV | 2 | 1.5 | 3 | 3 | 0 | 0 |
| | TREM1 | | | | | | | | |
| | Antibody | | | | | | | | |
| | Agent | | | | | | | | |
| | clone A | | | | | | | | |
| 3 | Anti- | 20 | IV | 2 | 10 | 4 | 3 | 0 | 0 |
| | TREM1 | | | | | | | | |
| | Antibody | | | | | | | | |
| | Agent | | | | | | | | |
| | clone A | | | | | | | | |
| 4 | Anti- | 50 | IV | 2 | 25 | 3 | 3 | 3 | 3 |
| | TREM1 | | | | | | | | |
| | Antibody | | | | | | | | |
| | Agent | | | | | | | | |
| | clone A | | | | | | | | |
| 5 | Anti- | 20 | SC | 0.1 | 50 | 3 | 3 | 0 | 0 |
| | TREM1 | | | | | | | | |
| | Antibody | | | | | | | | |
| | Agent | | | | | | | | |
| | clone A | | | | | | | | |

The following parameters and endpoints were evaluated in this study: mortality, clinical observations, body weights, qualitative food consumption, injection site observations, sex differences, ophthalmic examinations, quantitative electrocardiology, qualitative electrocardiology, neurologic examination, respiration rates, clinical pathology parameters (hematology, coagulation, clinical chemistry, and urinalysis), immunophenotyping, cytokine parameters, organ weights, and macroscopic and microscopic examinations. For all these assessed parameters, no statistically relevant perturbations were observed following once weekly IV administration of anti-TREM1 antibody agent clone A for 26 weeks. The presence of anti-drug antibodies, and bioanalysis and toxicokinetic parameters were also assessed in dosed animals and further described below.

Systemic exposure to anti-TREM1 antibody agent clone A was independent of sex following intravenous or subcutaneous administration of anti-TREM1 antibody agent clone A. For intravenous bolus administration the increase in CO on Days 1 and 85 was approximately proportional to dose and the increase in AUCₜₗₐₛₜ was less than dose proportional. On Day 176, the increase in both C₀ and AUCₜₗₐₛₜ was less than dose proportional. Systemic exposure (C₀ and AUC₀₋₁₆₈ₕᵣ values) to anti-TREM1 antibody agent clone A were slightly higher on Days 85 and 176 compared to Day 1. Exposure was similar between Days 85 and 176. In a single animal from Group 2) (3 mg/kg/dose), there was evidence of the presence of anti-TREM1 antibody agent clone A ADAs resulting in a reduced exposure to anti-TREM1 antibody agent clone A on Day 85.

In all animals that were administered anti-TREM1 antibody agent clone A by the SC route, anti-TREM1 antibody agent clone A was detected through the dose interval (168 hours postdose) in all animals on Days 1, 85, and 176, and prior to dose administration on Days 29, 57, 85, 113, 141, and 176 with the exception of a single animal on Day 176. For subcutaneous administration the exposure to anti-TREM1 antibody agent clone A (as measured by Cmax and AUC₀₋₁₆₈ₕᵣ) was generally higher on Day 85 or Day 176 compared to Day 1 for all animals with the exception of the single animal on Day 176.

In conclusion, administration of anti-TREM1 antibody agent clone A by intravenous (slow bolus) or subcutaneous injection once weekly for 26 weeks was well tolerated in cynomolgus monkeys up to the level of the 50 mg/kg/dose intravenous and 20 mg/kg/dose subcutaneous, and there were no noteworthy toxicological findings observed in the animals. Based on the results under the conditions of this example, the no-observed-adverse-effect level (NOAEL) was considered to be 50 mg/kg/dose and 20 mg/kg/dose for intravenous and subcutaneous routes, respectively. At the NOAEL, the gender combined mean C₀ and AUC₀₋₁₆₈ₕᵣ values on Day 176 for IV administration were 1530 μg/mL and 52600 g·hr/mL, and the gender combined mean Cₘₐₓ and AUC₀₋₁₆₈ₕᵣ values on Day 176 for subcutaneous administration were 143 μg/mL and 17300 μg·hr/mL, respectively.

Bioanalysis and Toxicokinetic Evaluations IV Bolus Administration of anti-TREM1 Antibody Agent Clone A

Systemic exposure to anti-TREM1 antibody agent clone A was independent of sex. Individual serum concentration-time profiles, C₀, and AUC values were similar between males and females (mean F:M C₀ ratios ranged from 0.844 to 1.21 and mean F:M AUC₀₋₁₆₈ₕᵣ ratios ranged from 0.843 to 1.34); therefore, the following discussion is based on data for males and females combined.

Anti-TREM1 antibody agent clone A was quantifiable up to 168 hours postdose on Day 1, 85, and 176, and up to 672, 1008, or 1512 hours postdose on Day 176 for recovery animals in Group 4. As a result of IV bolus administration, peak anti-TREM1 antibody agent clone A serum concentrations were estimated at time zero (C₀).

Following once weekly IV bolus (Groups 2, 3, 4) administration of anti-TREM1 antibody agent clone A to cynomolgus monkeys, mean C₀ and AUC₀₋₁₆₈ₕᵣ values of anti-TREM1 antibody agent clone A increased with increasing dose across the dose range on Days 1, 85, and 176. On Days 1 and 85, the increase in C₀ was approximately proportional to dose, and the increase in AUC₀₋₁₆₈ₕᵣ was less than dose proportional. On Day 176, the increase in both C₀ and AUC₀₋₁₆₈ₕᵣ was less than dose proportional. Systemic exposure (C₀ and AUC₀₋₁₆₈ₕᵣ values) to anti-TREM1 antibody agent clone A were slightly higher on Days 85 and 176 compared to Day 1.

TABLE 19
Summary (Mean ± SD) Toxicokinetic Parameters of anti-TREM1 antibody agent clone A in Sex Combined Cynomolgus
Monkey Serum Following IV Bolus (Groups 2, 3, 4) Administration of Anti-TREM1 Antibody Agent Clone A

| | Dose | C [symbol] | C [symbol] /Dose | t [symbol] | AUC [symbol] | AUC [symbol] /Dose | | |
|---|---|---|---|---|---|---|---|---|
| Day | (mg/kg) | (μg/mL) | (μg/mL/[mg/kg]) | (hr) | (hr*μg/mL) | (hr*μg/mL/[mg/kg]) | RC [symbol] | RAUC [symbol] |
| 1 | 3 | 103 ± 14.7 | 34.2 ± 4.90 | 168 | 5500 ± 635 | 1830 ± 212 | NA | NA |
| | | | | (168-168) | | | | |
| 85 | 3 | 142 ± 18.9 | 47.5 ± 6.29 | 168 | 9130 ± 2920 | 3040 ± 973 | 1.42 ± 0.320 | 1.71 ± 0.605 |
| | | | | (168-168) | | | | |
| 176 | 3 | 123 ± 16.5 | 40.9 ± 5.49 | 168 | 7930 ± 1580 | 2640 ± 525 | 1.20 ± 0.184 | 1.47 ± 0.370 |
| | | | | (168-168) | | | | |
| 1 | 20 | 549 ± 77.9 | 27.4 ± 3.90 | 168 | 22700 ± 2970 | 1140 ± 148 | NA | NA |
| | | | | (168-168) | | | | |
| 85 | 20 | 791 ± 61.3 | 39.6 ± 3.06 | 168 | 35700 ± 6750 | 1790 ± 338 | 1.41 ± 0.207 | 1.53 ± 0.109 |
| | | | | (168-168) | | | | |
| 176 | 20 | 719 ± 77.3 | 36.0 ± 3.86 | 168 | 32800 ± 7810 | 1640 ± 390 | 1.29 ± 0.204 | 1.40 ± 0.167 |
| | | | | (168-168) | | | | |
| 1 | 50 | 1510 ± 149 | 30.1 ± 2.97 | 168 | 40300 ± 4990 | 806 ± 99.8 | NA | NA |
| | | | | (168-168) | | | | |
| 85 | 50 | 1710 ± 237 | 34.1 ± 4.73 | 168 | 56400 ± 8370 | 1130 ± 167 | 1.14 ± 0.189 | 1.40 ± 0.145 |
| | | | | (168-168) | | | | |
| 176 | 50 | 1530 ± 176 | 30.7 ± 3.53 | 420 | 52600 ± 10300 | 1050 ± 205 | 1.03 ± 0.144 | 1.31 ± 0.225 |
| | | | | (168-1512) | | | | |

NA = Not Applicable
^{a}= Median (minimum-maximum)
^{b}= RC₀ = C₀ Day 85 or 176C₀ Day 1
^{c}= RAUC = AUC_{0-168 hr} Day 85 or 176AUC_{0-168 hr} Day 1 [symbol]
indicates data missing or illegible when filed

### Bioanalysis and Toxicokinetic Evaluations—Subcutaneous Administration of Anti-TREM1 Antibody Agent Clone A

Systemic exposure to anti-TREM1 antibody agent clone A was independent of sex. Individual serum concentration-time profiles, Cₘₐₓ, and AUC values were similar between males and females (mean F:M Cₘₐₓ ratios ranged from 0.868 to 1.25 and mean F:M AUC₀₋₁₆₈ₕᵣ ratios ranged from 0.707 to 1.09); therefore, the following discussion is based on data for males and females combined.

Anti-TREM1 antibody agent clone A was detected through the dose interval (168 hours postdose) in all animals on Days 1, 85, and 176, and prior to dose administration on Days 29, 57, 85, 113, 141, and 176 with the exception of a single animal on Day 176. In this animal anti-TREM1 antibody agent clone A was not detectable prior to dose administration on Day 176 and at 144- and 168-hours postdose. However, anti-TREM1 antibody agent clone A ADAs could not be detected in this animal.

The median tₘₐₓ values for anti-TREM1 antibody agent clone A in serum were observed by 24 hours postdose on Days 1, 85, and 176 (individual tmax values for anti-TREM1 antibody agent clone A ranged from 24 to 72 hours on Day 1, were all 24 hours on Day 85 and ranged from 6 to 24 hours on Day 176).

The exposure to anti-TREM1 antibody agent clone A (as measured by Cₘₐₓ and AUC₀₋₁₆₈ₕᵣ) was similar or higher on Day 85 or Day 176 compared to Day 1 for all animals with the exception of the single animal on Day 176. For animal 5503 the Cₘₐₓ and AUC were higher on Day 1 compared to Day 176.

TABLE 20
Summary (Mean ± SD) Toxicokinetic Parameters of anti-TREM1 antibody
agent clone A in Sex Combined Cynomolgus Monkey Serum Following
SC (Group 5) Administration of Anti-TREM1 Antibody Agent Clone A

| | Dose | Cₘₐₓ | tₘₐₓ [symbol] | t [symbol] | AUC [symbol] | | |
|---|---|---|---|---|---|---|---|
| Day | (mg/kg) | (μg/mL) | (hr) | (hr) | (hr*μg/mL) | RCₘₐₓ^{b} | RAUC [symbol] |
| 1 | 20 | 135 ± 24.2 | 24.0(24.0- | 168(168- | 15900 ± 1260 | NA | NA |
| | | | 72.0) | 168) | | | |
| 85 | 20 | 199 ± 40.5 | 24.0(24.0- | 168(168- | 25300 ± 4710 | 1.49 ± 0.301 | 1.59 ± 0.219\| |
| | | | 24.0) | 168) | | | |
| 176 | 20 | 143 ± 36.3 | 24.0(6.00- | 168(72.0- | 17300 ± 6800 | 1.08 ± 0.318 | 1.10 ± 0.426 |
| | | | 24.0) | 168) | | | |

NA = Not Applicable
^{a}= Median (minimum-maximum)
^{b}= RC₀ = C₀ Day 85 or 176C₀ Day 1
^{c}= RAUC = AUC_{0-168 hr} Day 85 or 176AUC_{0-168 hr} Day 1 [symbol]
indicates data missing or illegible when filed

### Anti-Drug Antibody—Anti-Anti-TREM1 Antibody Agent Clone a Antibody Evaluation Following IV Bolus Administration

Anti-anti-TREM1 antibody agent clone A antibodies were detected in 4 out of 114 (3.5%) control group (Group 1) samples collected from 12 control animals at pretreatment and on Day 1 predose, Day 29 predose, Day 57 predose, Day 85 predose, Day 113 predose, Day 141 predose, Day 176 predose, Day 176 at 168 hours postdose, and Day 176 at 1512 hours postdose. The antibodies were detected in samples that were obtained from different animals in samples collected prior to the administration of the first dose. Although anti-TREM1 antibody agent clone A ADAs were detected in the 4 samples, a titer value could not be obtained (i.e., the titer was less than the minimum required dilution).

Anti-TREM1 antibody agent clone A ADAs were detected in 3 out of 25 (12%) pretreatment samples from groups 2 (2 animals), 3 (1 animal), and 4 (0 animals). However, a titer value could not be obtained (i.e., the titer was less than the minimum required dilution).

Anti-TREM1 antibody agent clone A ADAs were detected in 6 out of 54 (11.1%) samples from Group 2 (3 mg/kg/dose). Two of these samples were pretreatment samples noted above. The remaining samples were obtained from an animal on Day 113 (titer=252), Day 141 (titer=399), Day 176 (titer=348), and Day 183 (titer=222). For this animal there was evidence of reduced exposure to anti-TREM1 antibody agent clone A (as measured by pre-dose anti-TREM1 antibody agent clone A concentrations) compared to other animals in the same dose group on Day 57 and 85, although anti-TREM1 antibody agent clone A ADAs could not be detected at those timepoints. On Day 113, Day 141, and Day 176 there was also evidence of reduced, exposure to anti-TREM1 antibody agent clone A and the presence of anti-TREM1 antibody agent clone A ADAs. Despite the presence of anti-TREM1 antibody agent clone A ADAs, the animals appeared to have been exposed to anti-TREM1 antibody agent clone A throughout the dose interval on Day 176 and anti-TREM1 antibody agent clone A was detectable prior to dose administration on Days 113, 141, and 176.

Anti-TREM1 antibody agent clone A ADAs were detected in 3 out of 52 (5.8%) samples from Group 3 (20 mg/kg/dose IV Bolus); however, a titer value could not be obtained (i.e., the titer was <the minimum required dilution). These positive results were obtained from 1 out of 7 animals pretreatment, 1 out of 7 animals on Day 29, and the terminal sample from an animal on Day 30. Anti-TREM1 antibody agent clone A ADAs were detected in 0 out of 114 (0%) samples from Group 4 (50 mg/kg/dose).

### Anti-Drug Antibody—Anti-Anti-TREM1 Antibody Agent Clone a Antibody Evaluation Following Subcutaneous Administration

All animals were negative for the presence of anti-TREM1 antibody agent clone A anti-drug antibodies (ADAs) pretreatment, and on Day 1 predose, Day 29 predose, Day 57 predose, Day 85 predose, Day 113 predose, Day 141 predose, Day 176 predose, Day 183 (Day 176 168 hours postdose), and Day 239 (Day 176 1512 hr postdose).

### Example 11: Anti-TREM1 Antibody Agent Clone a Safety, Tolerability, Pharmacokinetic, and Immunogenicity Analysis in Human Subjects

This example describes experiments analyzing the safety, tolerability, pharmacokinetic, and immunogenicity profile of a single IV dose of Anti-TREM1 antibody agent clone A in healthy adult human subjects.

Six cohorts of eight subjects each (6 treated with anti-TREM1 antibody agent clone A and 2 placebo) were evaluated and treated with provisional sequential doses of 0.015, 0.05 mg/kg, 0.15 mg/kg, 0.5 mg/kg, 1.5 mg/kg, and 4.5 mg/kg. In each cohort, subjects received a single IV infusion over 60 minutes of anti-TREM1 antibody agent clone A or placebo.

Safety (i.e., adverse events (AEs), clinical laboratory tests, vital signs, 12-lead safety electrocardiograms (ECGs), infusion site reaction, and physical examinations) were assessed throughout the study. Blood samples were collected for PK, immunogenicity (i.e., ADAs), and pharmacodynamic assessments of anti-TREM1 antibody agent clone A prior to the start of infusion and for up to Day 85 (±5 days) postdose.

Anti-TREM1 antibody agent clone A was safe and well-tolerated at single doses up to 4.5 mg/kg administered intravenously with no significant safety findings. All AEs were reported as mild in intensity with similar incidence of AEs across active and placebo groups. The most common AE reported was headache, which was reported with similar frequency between anti-TREM1 antibody agent clone A-treated subjects and placebo.

The PK profile of anti-TREM1 antibody agent clone A showed that anti-TREM1 antibody agent clone A concentrations increase with dose. Increases in Cₘₐₓ were approximately dose-proportional. Target-mediated drug disposition (TMDD) was apparent at the lower concentrations.

Receptor (i.e., TREM1) occupancy and soluble TREM1 (sTREM1) data demonstrated target engagement by anti-TREM1 antibody agent clone A.

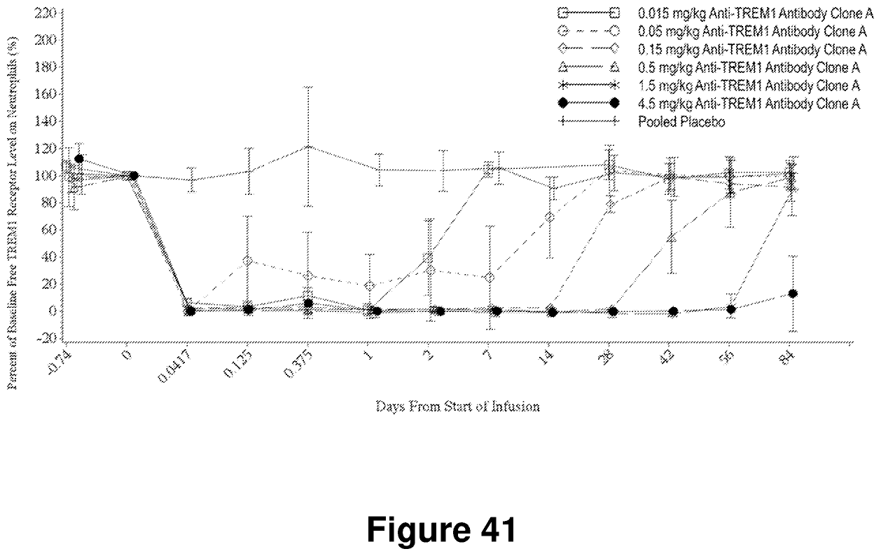

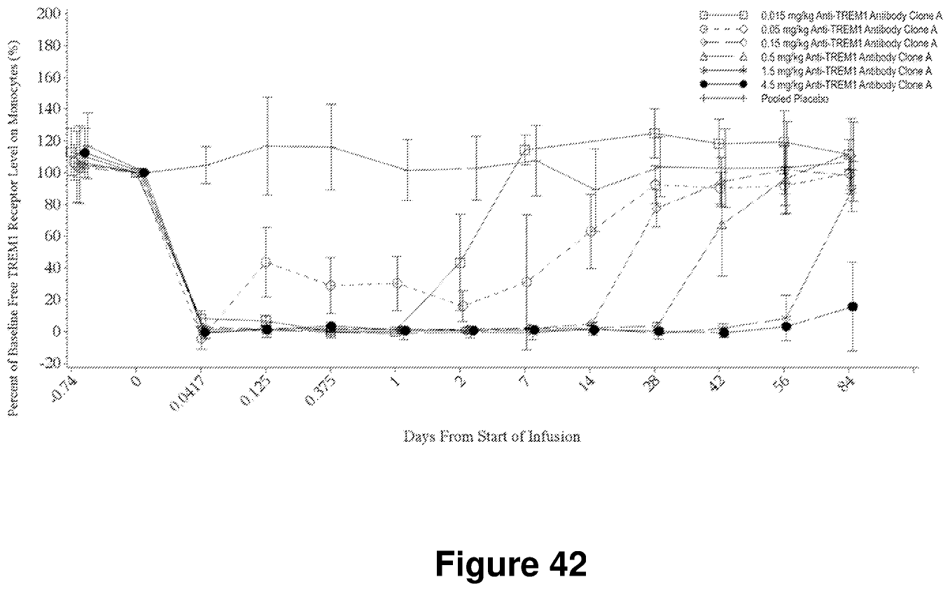

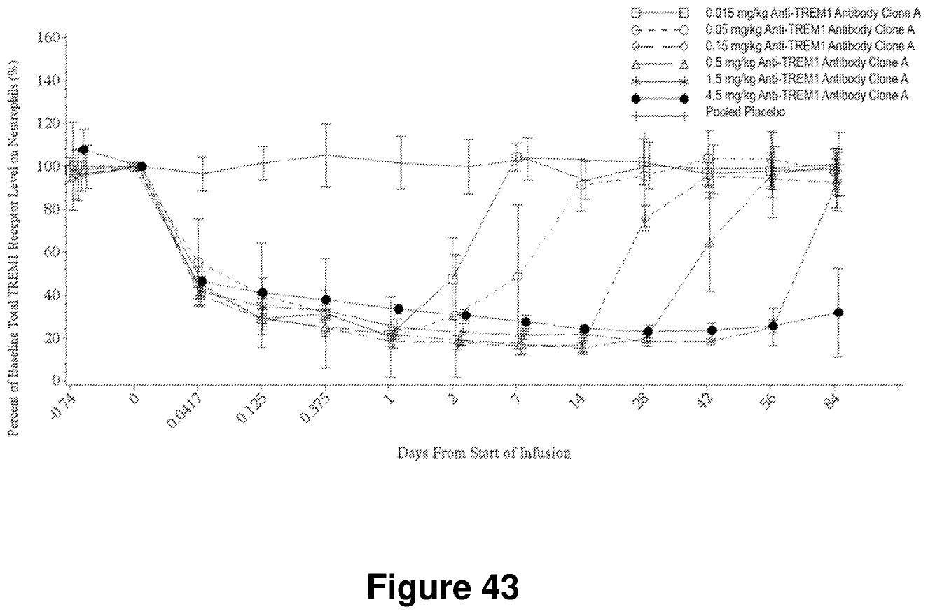

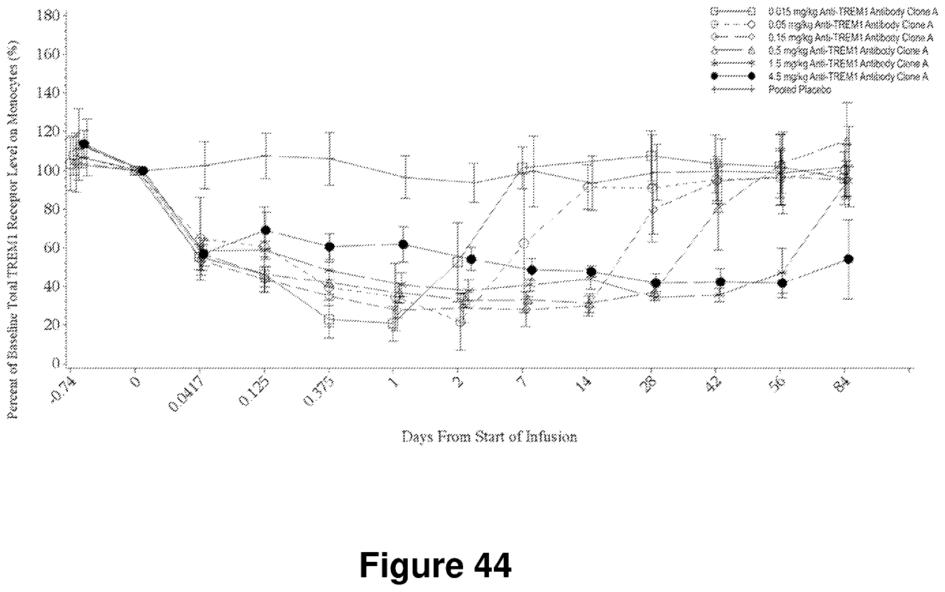

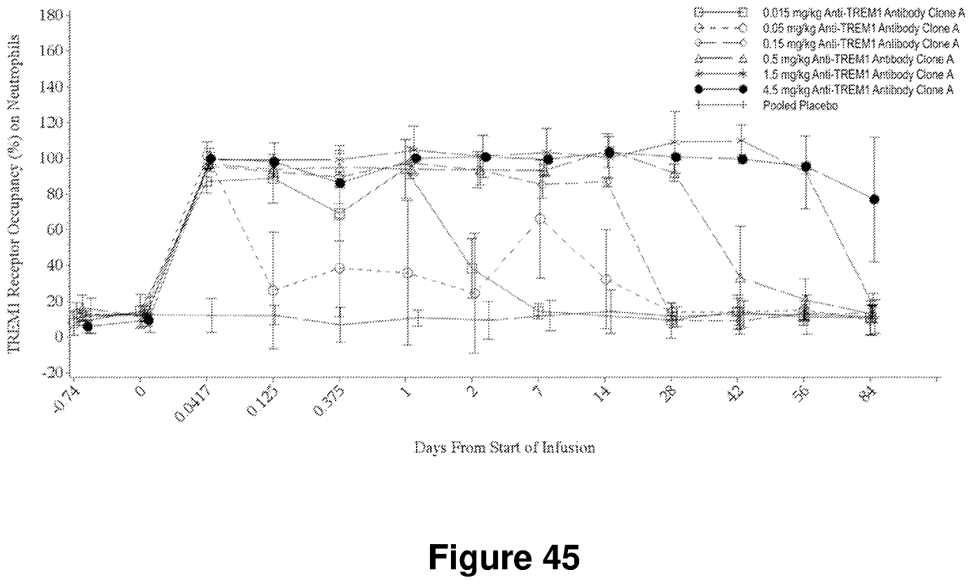

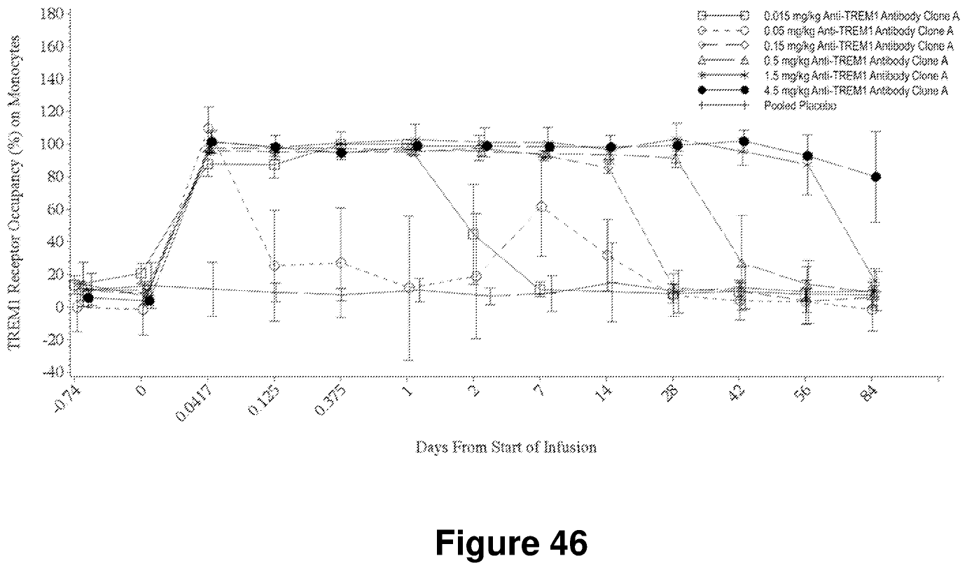

As shown in FIGS. 41-44, single doses of anti-TREM1 antibody agent clone A resulted in rapid and complete loss of free TREM1 receptor on both neutrophils (FIG. 41) and monocytes (FIG. 42). Pronounced reductions in free TREM1 receptor levels on monocytes and neutrophils were observed following a single IV infusion of 0.015 to 4.5 mg/kg anti-TREM1 antibody agent clone A; these treatment-related reductions generally return to baseline after anti-TREM1 antibody agent clone A was no longer detectable in serum. In addition, total TREM1 levels decreased after administration of anti-TREM1 antibody agent clone A (FIGS. 43 and 44). Without wishing to be bound by any particular theory, these results are consistent with anti-TREM1 antibody agent clone A's dual mechanism of action, and returned to baseline after anti-TREM1 antibody agent clone A was no longer detectable in serum. Full receptor occupancy, a measure of free and total TREM1 receptor, was achieved for at least 4 weeks at anti-TREM1 antibody agent clone A dose levels ≥0.5 mg/kg (FIGS. 45 and 46).

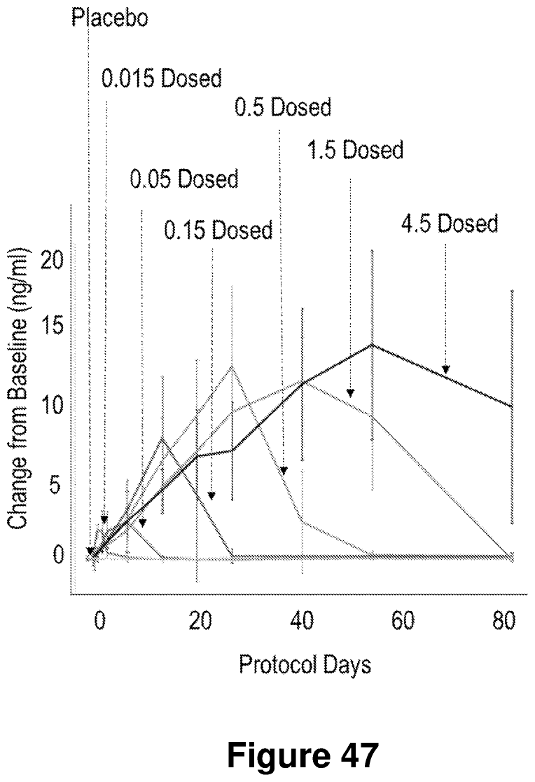

As shown in FIG. 47, single anti-TREM1 antibody agent clone A doses resulted in linear increases of total sTREM1 levels which return to baseline when anti-TREM1 antibody agent clone A is no longer detectable. Again, without wishing to be bound by any particular theory, these data suggest stabilization of sTREM1 when bound to anti-TREM1 antibody agent clone A.

### Example 12: Anti-TREM1 Antibody Agent Clone A Stability

This example describes experiments to evaluate the stability of anti-TREM1 antibody agent clone A drug substance (both non-good manufacturing practice (GMP) and GMP produced lots) stored at −70° C. or 5° C. over 12 months.

Anti-TREM1 antibody agent clone A drug substance (both non-GMP and GMP lots) had consistent stability when stored at −70° C. over a 12 month duration as assayed using rCE-SDS (purity analysis), imaged capillary isoelectric focusing (icIEF), size exclusion chromatography (SEC; purity analysis), and binding ELISA (potency analysis) assays. Similarly, anti-TREM1 antibody agent clone A drug substance (both non-GMP and GMP lots) had consistent stability when stored at 5° C. over a 12 month duration as assayed using rCE-SDS (purity analysis), size exclusion chromatography (SEC; purity analysis), and binding ELISA (potency analysis) assays.

Stability acceptance criteria for each assay were as follows: rCE-SDS≥90.0% purity and ≤10.0% total minor species, SEC≥95.0% Main, icIEF≥40% Main and ≤5.0% HMW, binding ELISA 60-140% relative potency.

Global fit was additionally used to project stability using a 95% confidence interval based on the 12-month data for anti-TREM1 antibody agent clone A drug substance and 9 month data for anti-TREM1 antibody agent clone A drug product. SEC projections indicated the anti-TREM1 antibody agent clone A drug substance will stay within the acceptance criteria after 60M of storage at −70° C. and anti-TREM1 antibody agent clone A drug product will likely remain within the main peak acceptance criteria after 60 months of storage at −70° C. (data not shown).

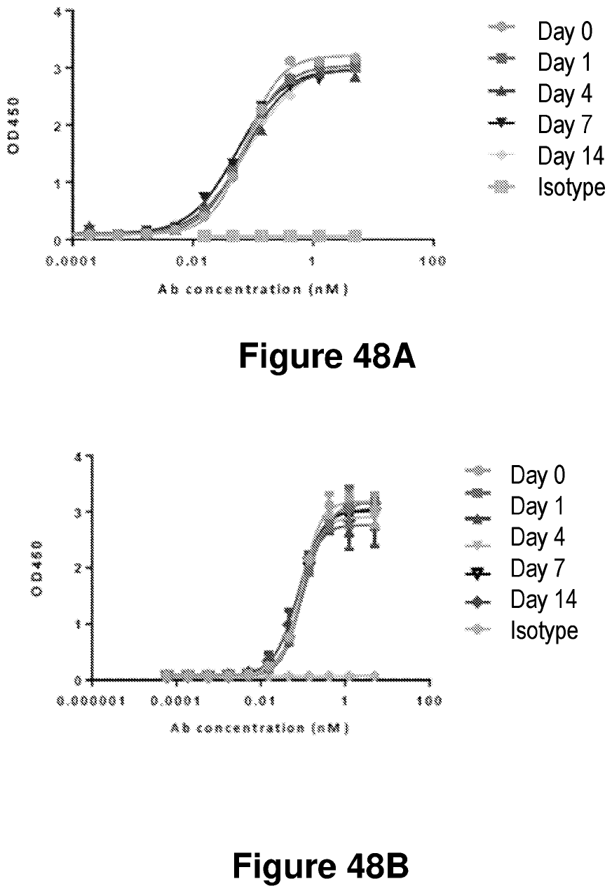

As further shown in FIGS. 48A-48B, anti-TREM1 antibody agent clone A is stable in human (FIG. 48A) and cynomolgus (FIG. 48B) serum for at least 14 days postdose.

1-100. (canceled)

101. An anti-triggering receptor expressed on myeloid cells 1 (TREM1) antibody wherein the antibody comprises:
a) a light chain variable region (VL) comprising the amino acid sequence of SEQ ID NO: 19 (LC CDR1); the amino acid sequence of GAS (LC CDR2); and the amino acid sequence of SEQ ID NO: 20 (LC CDR3); and
b) a heavy chain variable region (VH) comprising the amino acid sequence of SEQ ID NO: 15 (HC CDR1); the amino acid sequence of SEQ ID NO: 16 (HC CDR2); and the amino acid sequence of SEQ ID NO: 17 (HC CDR3).

102. The anti-TREM1 antibody of claim 101, wherein the antibody comprises:
a) a VL comprising a sequence at least 90% identical to the amino acid sequence of SEQ ID NO: 18; and
b) a VH comprising a sequence at least 90% identical to the amino acid sequence of SEQ ID NO: 14.

103. The anti-TREM1 antibody of claim 102, wherein the antibody comprises:
a) a VL comprising the amino acid sequence of SEQ ID NO: 18; and
b) a V_{H} comprising the amino acid sequence of SEQ ID NO: 14.

104. The anti-TREM1 antibody of claim 101, wherein the antibody comprises:
a) a light chain (LC) comprising a sequence at least 90% identical to the amino acid sequence of SEQ ID NO: 33; and
b) a heavy chain (HC) comprising a sequence at least 90% identical to the amino acid sequence of SEQ ID NO: 31.

105. The anti-TREM1 antibody of claim 104, wherein the antibody comprises:
a) a light chain (LC) comprising the amino acid sequence of SEQ ID NO: 33; and
b) a heavy chain (HC) comprising the amino acid sequence of SEQ ID NO: 31.

106. The anti-TREM1 antibody of claim 101, wherein the antibody comprises:
a) a kappa LC constant region;
b) a lambda LC constant region;
c) an immunoglobulin isotype selected from the group consisting of IgA, IgD, IgE, IgG, and IgM;
d) an Fc domain;
e) a human IgG Fc domain, optionally wherein the IgG constant region comprises a PVAdelG mutation; and/or
f) an antibody-drug conjugate (ADC).

107. A nucleic acid having a nucleotide sequence that encodes the antibody of claim 101.

108. A vector comprising the nucleic acid of claim 107.

109. A host cell comprising the vector of claim 108, optionally wherein the host cell is a yeast cell, a bacterial cell, a mammalian cell, or an insect cell.

110. A method of making an anti-TREM1 antibody, the method comprising: culturing the host cell of claim 109 under conditions wherein the anti-TREM1 antibody is expressed by said host cell.

111. A pharmaceutical composition comprising an anti-TREM1 antibody of claim 101, and a pharmaceutically acceptable carrier.

112. A method of treating a disease, disorder, or condition associated with a TREM1-mediated immune response in a subject, the method comprising administering to the subject an anti-TREM1 antibody of claim 101, optionally wherein the subject is a human.

113. The method of claim 112, wherein the disease, disorder, or condition is an inflammatory disease, disorder, or condition, optionally wherein the inflammatory disease, disorder, or condition is selected from the group consisting of an inflammatory bowel disease (IBD), intestinal IBD, an extra-intestinal manifestation (EIM) of IBD, Crohn's disease, ulcerative colitis (UC), sepsis, a fibrotic disease, rheumatoid arthritis, chronic obstructive pulmonary disease (COPD), acute respiratory distress syndrome (ARDS), COVID-19, Post-MI (ischemic reperfusion), atherosclerosis, acute stroke (ischemic reperfusion), stroke (hemorrhagic), renal ischemia reperfusion injury, pancreatitis, renal fibrosis, liver fibrosis, NASH, Sickle cell vaso-occlusive crisis, Marfan syndrome, HIV infection, motor neurogenerative disorders, periodontal/gingivitis, cancers, diabetic foot ulcers, gout, lupus, psoriasis, an arthropathy (e.g., arthritis or synovitis), and Behcet's disease.

114. The method of claim 112, wherein the subject has received, or is receiving, an alternative therapy, optionally wherein the alternative therapy is an alternative IBD therapy.

115. The method of claim 114, wherein the alternative therapy is or comprises an anti-TNFα therapy (e.g., infliximab, adalimumab, golimumab, and certolizumab pegol); a JAK inhibitor therapy (including selective and non-selective inhibitors) (e.g., upadacitinib, tofacitinib and filgotinib); an anti-integrin therapy (e.g., vedolizumab and natalizumab); an anti-IL-23 therapy (e.g., guselkumab, mirikizumab, and brazikumab); an anti-IL-12/23 therapy (e.g., ustekinumab); an anti-IL-23A therapy (e.g., risankizumab); an S1PR agonist or modulator therapy (e.g., ozanimod and etrasimod); a 5-aminosalicylate therapy (e.g., mesalamine, olsalazine, balsalazide, and sulfasalazine); an immunomodulator therapy (e.g., azathioprine, 6-mercaptopurine, and methotrexate); a corticosteroid therapy (e.g., prednisone, methylprednisolone, hydrocortisone, and budesonide); an anti-TL1A therapy (e.g., PRA023 and RVT-3101); a kinase inhibitor therapy (e.g., ritlecitinib), a TYK2 inhibitor therapy (e.g., deucravacitinib), an anti-IL-36 therapy (e.g., spesolimab); an anti-IL-13 therapy (e.g., dupilumab), a miR-124 upregulator therapy (e.g., obefazimod), a TLR9 agonist therapy (e.g., cobitolimod); or a combination thereof.

116. A method of administering an anti-TREM1 antibody of claim 101 to a subject in need thereof expressing a biomarker that:
a) is or is a proxy for an elevated level of neutrophils and/or monocytes (e.g., inflammatory monocytes), optionally, wherein the biomarker is or is a proxy for an elevated level of activated neutrophils;
b) is or comprises one or more product(s) of neutrophils and/or monocytes (e.g., inflammatory monocytes), wherein the biomarker has been correlated with responsiveness to the anti-TREM1 antibody of claim 101; and/or
c) is indicative of TREM1 level and/or activity, optionally wherein the subject is a human.

117. The method of claim 116, wherein the biomarker is:
i) a cell-based biomarker, optionally wherein the biomarker is monocyte and/or neutrophil count, TREM1 RNA present in monocytes and/or neutrophils, TREM1 present in monocytes and/or neutrophils, or TREM1 present on the cell surface of monocytes and/or neutrophils;
ii) a tissue biomarker, optionally wherein the biomarker is mucosal ulceration, neutrophils in lamina propria and/or epithelial cells, or lymphocytes, plasmocytes, and/or eosinophils in lamina propria;
iii) a product produced by activated neutrophils, optionally wherein the biomarker is calprotectin, PGLYRP1, or soluble TREM1;
iv) a product produced by inflammatory monocytes, optionally wherein the biomarker is soluble TREM1;
v) a TREM1 ligand, optionally wherein the TREM1 ligand is bacterial peptidoglycan (PGN) and/or PGLYRP1;
vi) a TREM1 gene activation signature;
vii) detectable in a sample selected from the group consisting of blood, disease tissue, fecal matter, components or fractions thereof, and combinations thereof;
viii) monocyte and/or neutrophil count and the biomarker is detect in disease tissue;
ix) TREM1 RNA present in monocytes and/or neutrophils and the biomarker is detected in disease tissue;
x) TREM1 present in monocytes and/or neutrophils and the biomarker is detected in disease tissue;
xi) TREM1 present on the cell surface of monocytes and/or neutrophils and the biomarker is detected in disease tissue;
xii) a tissue biomarker and the biomarker is detected in disease tissue;
xiii) soluble TREM1 and the biomarker is detected in blood and/or fecal matter;
xiv) calprotectin and the biomarker is detected in blood and/or fecal matter;
xv) bacterial PGN and the biomarker is detected in fecal matter;
xvi) PGLYRP1 and the biomarker is detected in blood and/or fecal matter;
xvii) TREM1 gene activation signature and the biomarker is detected in disease tissue and/or blood; and/or
xviii) combinations of i) through xviii) thereof.

118. A method of inhibiting TREM1, the method comprising contacting a cell, tissue, or subject with the anti-TREM1 antibody of claim 101, optionally wherein the subject is a human.

119. A method of antagonizing TREM1-mediated inhibition of maturation of monocytes to macrophages comprising contacting a cell, tissue, or subject with the anti-TREM1 antibody of claim 101, optionally wherein the subject is a human.
